(19) United States
(12) Patent Application Publication
(10) Pub. No.: US 2005/0153903 A1
(43) Pub. Date: Jul. 14, 2005

(54) Methods of preparing compounds useful as protease inhibitors

(75) Inventors: David John Kucera, Del Mar, CA (US); Nabil Lauze Saeed, San Diego, CA (US); Robert William Scott, San Diego, CA (US)

Correspondence Address: AGOURON PHARMACEUTICALS, INC., 10350 NORTH TORREY PINES ROAD, LA JOLLA, CA, 92037, US

(73) Assignee: Agouron Pharmaceuticals, Inc.

(21) Appl. No.: 11/003,948

(22) Filed: Dec. 3, 2004

Related U.S. Application Data
(60) Provisional application No. 60/527,470, filed on Dec. 4, 2003.
(60) Provisional application No. 60/591,354, filed on Jul. 26, 2004.

(51) Int. Cl. A61K 38/04; C07K 5/04

(52) U.S. Cl. 514/19; 548/537

(57) ABSTRACT

The invention relates to methods of preparing compounds of formula (I) 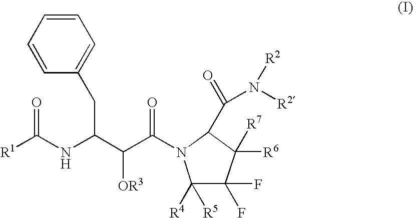 that are useful as inhibitors of the HIV protease enzyme. The present invention also relates to intermediate compounds useful in the preparation of compounds of formula (I).

This application claims priority under 35 U.S.C. § 119(e) to U.S. Provisional Application No. 60/527,470, filed Dec. 4, 2003, and 60/591,354, filed Jul. 26, 2004, both of which are hereby incorporated by reference.

### FIELD OF THE INVENTION

The present invention concerns methods of preparing compounds useful as inhibitors of the HIV protease enzyme, intermediates in the preparation of such compounds, as well as crystal forms of such compounds.

### BACKGROUND

The present invention relates to methods of preparing and intermediate compounds useful in the preparation of inhibitors of the human immunodeficiency virus (HIV) protease.

Acquired Immune Deficiency Syndrome (AIDS) causes a gradual breakdown of the body's immune system as well as progressive deterioration of the central and peripheral nervous systems. Since its initial recognition in the early 1980's, AIDS has spread rapidly and has now reached epidemic proportions within a relatively limited segment of the population. Intensive research has led to the discovery of the responsible agent, human T-lymphotropic retrovirus III (HTLV-III), now more commonly referred to as HIV.

HIV is a member of the class of viruses known as retroviruses and is the etiologic agent of AIDS. The retroviral genome is composed of RNA which is converted to DNA by reverse transcription. This retroviral DNA is then stably integrated into a host cell's chromosome and, employing the replicative processes of the host cells, produces new retroviral particles and advances the infection to other cells. HIV appears to have a particular affinity for the human T-4 lymphocyte cell that plays a vital role in the body's immune system. HIV infection of these white blood cells depletes this white cell population. Eventually, the immune system is rendered inoperative and ineffective against various opportunistic diseases such as, among others, pneumocystic carini pneumonia, Kaposi's sarcoma, and cancer of the lymph system.

Although the exact mechanism of the formation and working of the HIV virus is not understood, identification of the virus has led to some progress in controlling the disease. For example, the drug azidothymidine (AZT) has been found effective for inhibiting the reverse transcription of the retroviral genome of the HIV virus, thus giving a measure of control, though not a cure, for patients afflicted with AIDS. The search continues for drugs that can cure or at least provide an improved measure of control of the deadly HIV virus and thus the treatment of AIDS and related diseases.

Retroviral replication routinely features post-translational processing of polyproteins. This processing is accomplished by virally encoded HIV protease enzyme. This yields mature polypeptides that will subsequently aid in the formation and function of infectious virus. If this molecular processing is stifled, then the normal production of HIV is terminated. Therefore, inhibitors of HIV protease may function as anti-HIV viral agents.

HIV protease is one of the translated products from the HIV structural protein pol 25 gene. This retroviral protease specifically cleaves other structural polypeptides at discrete sites to release these newly activated structural proteins and enzymes, thereby rendering the virion replication-competent. As such, inhibition of the HIV protease by potent compounds may prevent proviral integration of infected T-lymphocytes during the early phase of the HIV-1 life cycle, as well as inhibit viral proteolytic processing during its late stage. Additionally, the protease inhibitors may have the advantages of being more readily available, longer lived in virus, and less toxic than currently available drugs, possibly due to their specificity for the retroviral protease.

Methods for preparing compounds useful as HIV protease inhibitors have been described in, e.g., U.S. Pat. No. 5,962,640; U.S. Pat. No. 5,932,550; U.S. Pat. No. 6,222,043; U.S. Pat. No. 5,644,028; WO 02/100844, Australian Patent No. 705193; Canadian Patent Application No. 2,179,935; European Patent Application No. 0 751 145; Japanese Patent Application No. 100867489; Y. Hayahsi, et al., J. Org. Chem., 66, 5537-5544 (2001); K. Yoshimura, et al., Proc. Natl. Acad. Sci. USA, 96, 8675-8680 (1999); and, T. Mimoto, et al., J. Med. Chem., 42, 1789-1802 (1999). Thus, methods of preparing compounds useful as protease inhibitors have previously been known. However, these methods were linear and thus inefficient. The improved methods of the invention provide for convergent synthetic routes having maximized efficiency.

### SUMMARY

The present invention relates to methods of preparing compounds of formula (I), or a salt or solvate thereof: 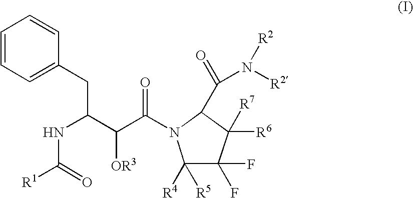
wherein:
R¹ is phenyl optionally substituted by at least one substituent independently chosen from C₁₋₆ alkyl, hydroxyl, C₁₋₆ alkylcarbonyloxy, C₆₋₁₀ arylcarbonyloxy, and heteroarylcarbonyloxy;
R² is C₂₋₆ alkenyl or C₁₋₆ alkyl optionally substituted with at least one halogen;
R^{2′} is H or C₁-C₄ alkyl;
R³ is hydrogen or a hydroxylprotecting group; and
R⁴, R⁵, R⁶ and R⁷ are independently selected from H and C₁-C₆ alkyl; comprising:
reacting a compound of formula (II), wherein Y¹ is hydroxyl or a leaving group and R¹ is as described for formula (I), with a compound of formula (III), or a salt or solvate thereof.

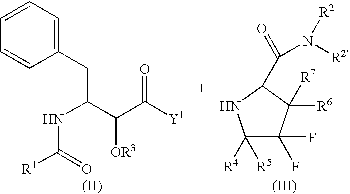

The present invention further comprises deprotecting the compound of formula (I) when R³ is a hydroxyl-protecting group to afford a compound of formula (I) wherein R³ is hydrogen.

The present invention also provides intermediate compounds that are useful for the preparation of compounds of formula (I).

The following describe further embodiments of the present invention.

In another aspect of the present invention are provided methods for preparing compounds of formula (I), 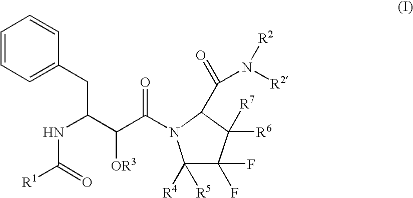
wherein:
R¹ is phenyl optionally substituted by at least one substituent independently chosen from C₁₋₆ alkyl, hydroxyl, C₁₋₆ alkylcarbonyloxy, C₆₋₁₀ arylcarbonyloxy, and heteroarylcarbonyloxy;
R² is C₂₋₆ alkenyl or C₁₋₆ alkyl optionally substituted with at least one halogen;
R^{2′} is H or C₁-C₄ alkyl;
R³ is a hydroxyl protecting group; and

R⁴, R⁵, R⁶ and R⁷ are independently selected from H and C₁-C₆ alkyl; comprising:
reacting a compound of formula (II), wherein Y¹ is hydroxyl or a leaving group, with a compound of formula (III), or a salt or solvate thereof.

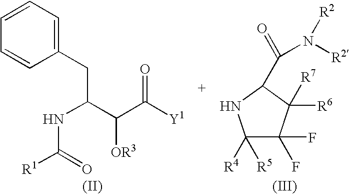

In another aspect of the present invention are provided methods for the preparation of compounds of formula (I), comprising:
(i) reacting a compound of formula (IV), wherein Y¹ is hydroxy or —OP¹, wherein P¹ is a suitable protecting group, and R³ is hydrogen, C₁-C₄ alkyl, or a suitable hydroxyl protecting group, with a compound of formula (V), wherein Y² is a leaving group, to afford a compound of formula (II); 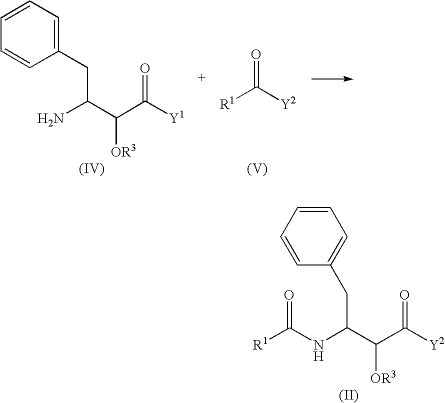
(ii) reacting the compound of formula (II) with a compound of formula (III), or a salt or solvate thereof, to afford a compound of formula (I); and 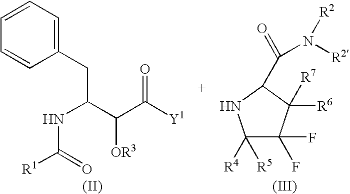
   (iii) optionally deprotecting those compounds of formula (I) wherein R³ is a hydroxyl protecting group, to afford a compound of formula (I) wherein R³ is hydrogen.

In another aspect of the present invention are provided any of the methods described herein of preparing the compounds of the formula (I) wherein in the compound of (II) Y¹ is hydroxyl.

In still another aspect of the present invention are provided any of the methods described herein for the preparation of compounds of formula (I), wherein:

R¹ is phenyl optionally substituted by at least one substituent independently chosen from C₁₋₆ alkyl, hydroxyl, C₁₋₆ alkylcarbonyloxy, C₆₋₁₀ arylcarbonyloxy, and heteroarylcarbonyloxy;
R² is C₂₋₆ alkenyl or C₁alkyl optionally substituted with at least one halogen;
R^{2′} is H, methyl, or ethyl;
R³ is a hydroxyl protecting group; and
R⁴, R⁵, R⁶ and R⁷ are independently chosen from H and C₁-C₆ alkyl.

Yet another aspect of the present invention provides any of the methods described herein for the preparation of compounds of formula (I), wherein:

R¹ is phenyl substituted with at least one substituent independently chosen from C₁₋₆ alkyl, hydroxyl, C₁₋₆ alkylcarbonyloxy, C₆₋₁₀, arylcarbonyloxy, and heteroarylcarbonyloxy;
R² is C₂₋₆ alkenyl or C₁ alkyl optionally substituted with at least one halogen;
R^{2′} is H, methyl, or ethyl;
R³ is C₁₋₆ alkylcarbonyl, C₆₋₁₀ arylcarbonyl, or heteroarylcarbonyl;
R⁴ and R⁵ are each H; and
R⁶ and R⁷ are independently chosen from H and methyl.

In yet another aspect of the present invention provides any of the methods described herein for the preparation of compounds of formula (I), wherein:
R¹ is phenyl substituted with at least one substituent independently chosen from C₁₋₆ alkyl, hydroxyl, C₁₋₆ alkylcarbonyloxy C₆₋₁₀ arylcarbonyloxy, and heteroarylcarbonyloxy;
R² is C₂₋₆ alkenyl or C₁₋₆ alkyl optionally substituted with at least one halogen;
R^{2′} is H;
R³ is C₁₋₆ alkylcarbonyl, C₆₋₁₀ arylcarbonyl, or heteroarylcarbonyl;
R⁴ and R⁵ are each H; and

R⁶ and R⁷ are independently chosen from H and methyl.

Another aspect of the present invention provides any of the methods described herein for the preparation of compounds of formula (I), wherein:
R¹ is phenyl substituted with at least one substituent independently chosen from methyl, hydroxyl, C₁₋₆ alkylcarbonyloxy, C₆₋₁₀ arylcarbonyloxy, and heteroarylcarbonyloxy;
R² is C₂₋₆ alkenyl or C₁₋₆ alkyl optionally substituted with at least one halogen;
R^{2′} is H;
R³ is C₁₋₆ alkylcarbonyl, C₆₋₁₀ arylcarbonyl, or heteroarylcarbonyl;
R⁴ and R⁵ are each H; and
R⁶ and R⁷ are methyl.

The present invention also provides any of the methods described herein for the preparation of compounds of formula (I), wherein:
R¹ is phenyl substituted with at least one substituent independently chosen from methyl, hydroxyl, C₁₋₆ alkylcarbonyloxy, C₆₋₁₀ arylcarbonyloxy, and heteroarylcarbonyloxy;
R² is C₂₋₆ alkenyl or C₁₋₆ alkyl optionally substituted with at least one fluorine;
R^{2′} is H;
R³ is C₁₋₆ alkylcarbonyl;
R⁴ and R⁵ are each H; and
R⁶ and R⁷ are methyl.

In yet another aspect of the present invention are provided any of the methods described herein for the preparation of compounds of formula (I), wherein:
R¹ is phenyl substituted with at least one substituent independently chosen from methyl, hydroxyl, C₁₋₆ alkylcarbonyloxy, C₆₋₁₀ arylcarbonyloxy, and heteroarylcarbonyloxy;
R² is C₁₋₆ alkyl optionally substituted with at least one fluorine;
R^{2′} is H;
R³ is C₁₋₆ alkylcarbonyl;
R⁴ and R⁵ are each H; and
R⁶ and R⁷ are methyl.

In still another aspect of the invention are provided any of the methods described herein for the preparation of compounds of formula (I), wherein:
R¹ is phenyl substituted with at least one substituent independently chosen from methyl, hydroxyl, and methylcarbonyloxy;
R² is C₁₋₆ alkyl substituted with at least one fluorine;
R^{2′} is H;
R³ is C₁₋₆ alkylcarbonyl;
R⁴ and R⁵ are each H; and
R⁶ and R⁷ are methyl.

In yet another aspect of the present invention are provided any of the methods described herein for the preparation of compounds of formula (I), wherein:
R¹ is phenyl substituted with at least one substituent independently chosen from methyl, hydroxyl, and methylcarbonyloxy;
R² is —CH₂CF₃;
R^{2′} is H;
R³ is methylcarbonyl;
R⁴ and R⁵ are each H; and
R⁶ and R⁷ are methyl.

In another aspect of the present invention are provided any of the methods described herein for the preparation of compounds of formula (I), wherein:
R¹ is phenyl substituted with at least one substituent independently chosen from methyl and methylcarbonyloxy;
R² is —CH₂CF₃;
R^{2′} is H;
R³ is methylcarbonyl;
R⁴ and R⁵ are each H; and
R⁶ and R⁷ are methyl.

Also in the present invention are provided any of the methods described herein for the preparation of compounds of formula (I), wherein the compound of formula (I) is:

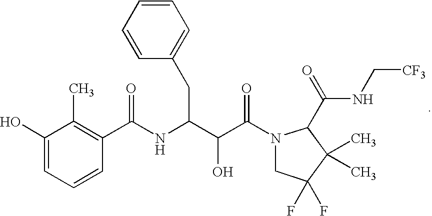

In the present invention are provided any of the methods described herein for the preparation of compounds of formula (I), wherein:
R¹ is phenyl substituted with at least one substituent independently chosen from methyl, hydroxyl, C₁₋₆ alkylcarbonyloxy, C₆₋₁₀ arylcarbonyloxy, and heteroarylcarbonyloxy;
R² is C₁₋₆ alkyl;
R^{2′} is H;
R³ is C₁₋₆ alkylcarbonyl;
R⁴ and R⁵ are each H; and
R⁶ and R⁷ are methyl.

In yet another aspect of the present invention are provided any of the methods described herein for the preparation of compounds of formula (I), wherein:
R¹ is phenyl substituted with at least one substituent independently chosen from methyl, hydroxyl, and methylcarbonyloxy;
R² is —CH₂CH₃;
R^{2′} is H;
R³ is methylcarbonyl;
R⁴ and R⁵ are each H; and
R⁶ and R⁷ are methyl.

Also provided in the present invention are any of the methods described herein for the preparation of compounds of formula (I), wherein:
R¹ is phenyl substituted with at least one substituent independently chosen from methyl and methylcarbonyloxy;
R² is —CH₂CH₃;
R^{2′} is H;
R³ is methylcarbonyl;
R⁴ and R⁵ are each H; and
R⁶ and R⁷ are methyl.

Also provided are any of the methods described herein of preparation for the compounds of formula (I), wherein the compound of formula (I) is:

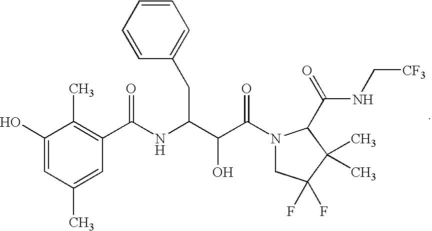

In still another aspect of the present invention are provided methods for the preparation of compounds of formula (I-C), 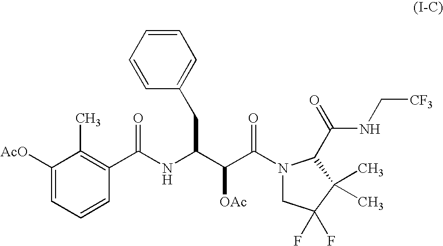
comprising:
reacting a compound of formula (II-A) with a compound of formula (III-B), or a salt or solvate thereof. 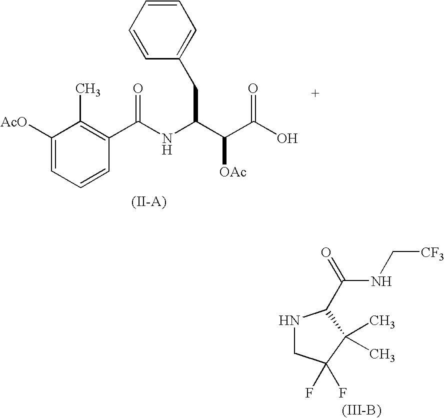
   Still another aspect of the present invention provides a method of preparing a compound of formula (I-C), 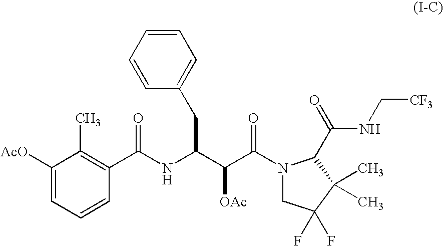
   comprising:
(i) reacting a compound of formula (IV-A) with a compound of formula (V-A), 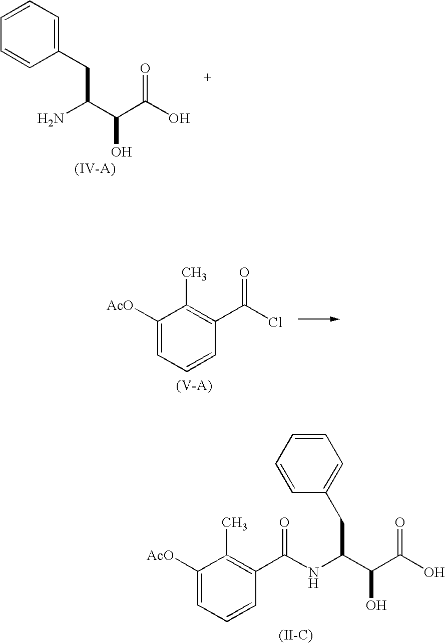
   to afford a compound of formula (II-C);
(ii) treating the compound of formula (II-C) with an acetylating agent to afford a compound of formula (II-A); and 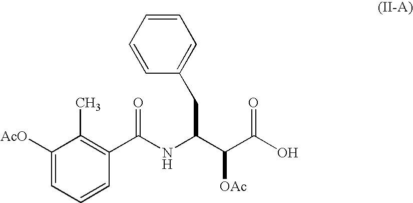
(iii) reacting the compound of formula (II-A) with a compound of formula (III-B).

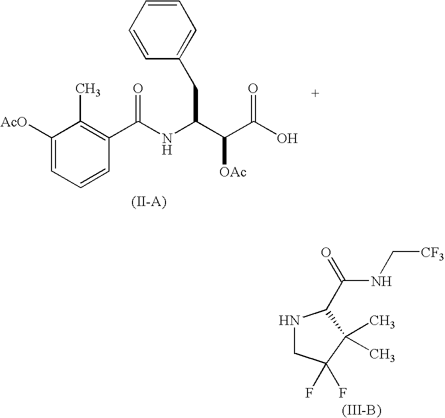

The present invention also provides methods for the preparation of compounds of formula (I-D), 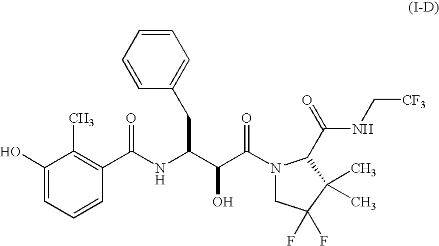
said method comprising:
(i) reacting a compound of formula (II-A) with a compound of formula (III-B), or a salt or solvate thereof, 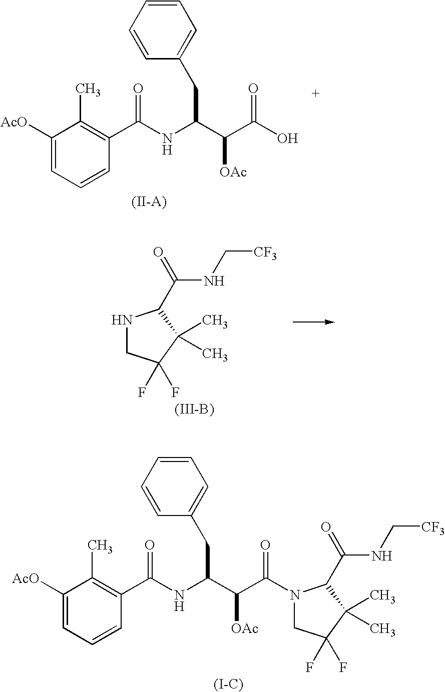
   to afford a compound of formula (I-C); and
(ii) deprotecting the compound of formula (I-C).

In yet another aspect of the present invention are provided methods for the preparation of compounds of formula (I-D), 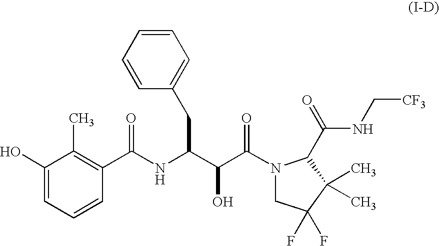
comprising:
(i) reacting a compound of formula (IV-A) with a compound of formula (V-A), 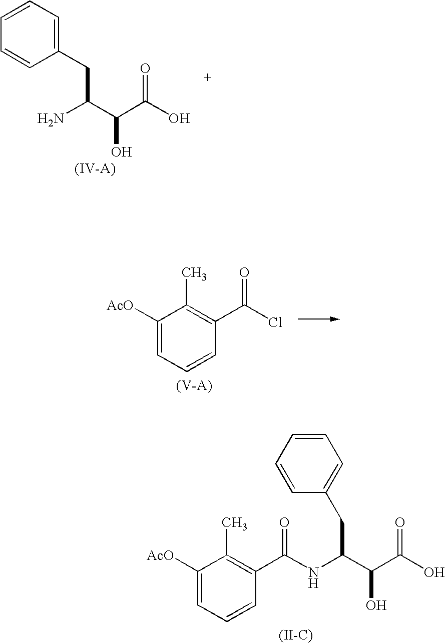
   to afford a compound of formula (II-C);
(ii) treating the compound of formula (II-C) with an acetylating agent to afford a compound of formula (II-A); and 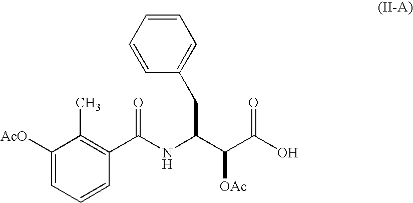
(iii) reacting the compound of formula (II-A) with a compound of formula (III-B), 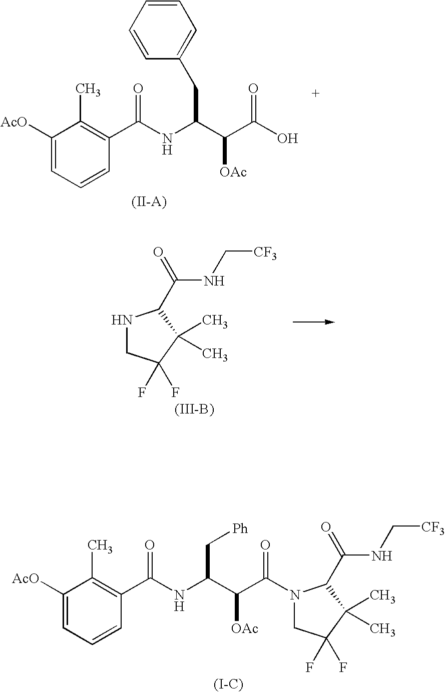
   to afford a compound of formula (I-C); and
(iii) deprotecting the compound of formula (I-C).

Another aspect of the present invention provides methods for the preparation of compounds of formula (I-E), 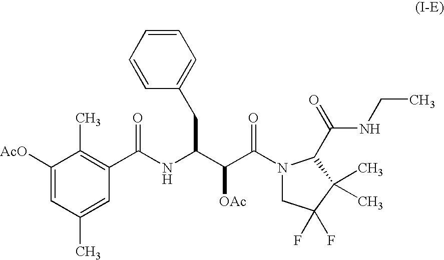
comprising:
reacting a compound of formula (II-B) with a compound of formula (III-C), or a salt or solvate thereof.

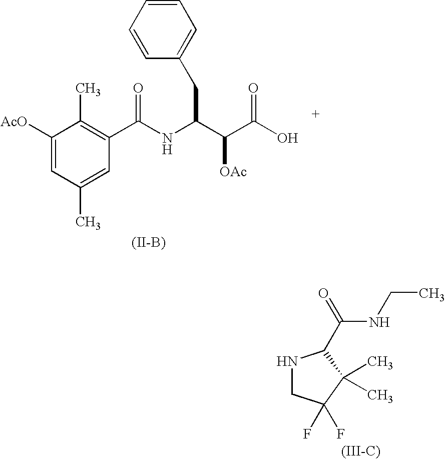

A still further aspect of the present invention provides methods for the preparation of compounds of formula (I-E), 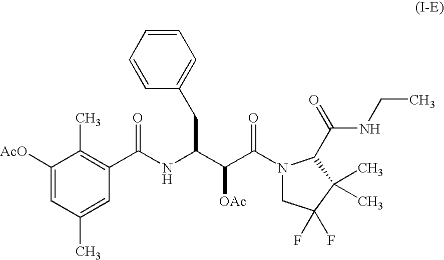
comprising:
(i) reacting a compound of formula (IV-A) with a compound of formula (V-B), 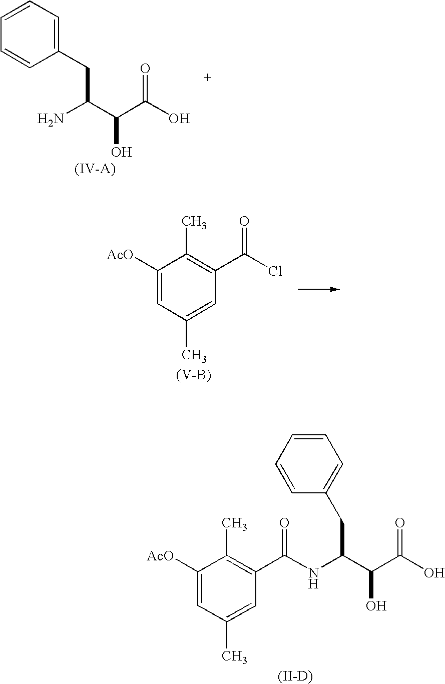
   to afford a compound of formula (II-D);
(ii) treating the compound of formula (II-D) with an acetylating agent, 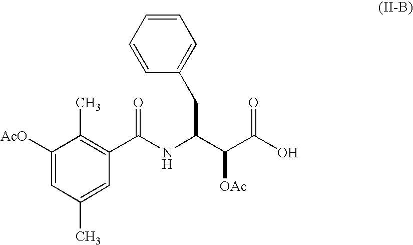
   to afford a compound of formula (II-B); and
(iii) reacting the compound of formula (II-B) with a compound of formula (III-C).

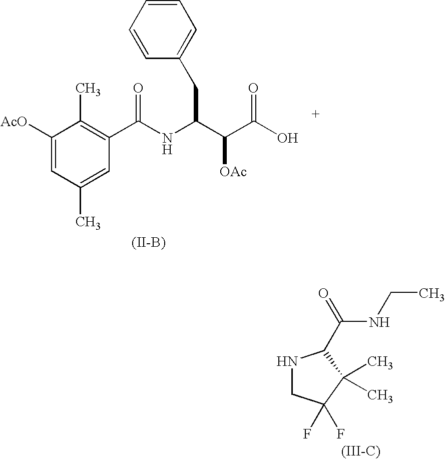

Also provided are methods for the preparation of compounds of formula (I-F), 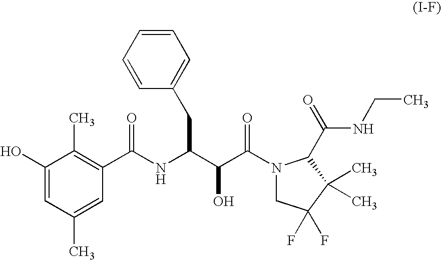
said method comprising:
(i) reacting a compound of formula (II-B) with a compound of formula (III-C), or a salt or solvate thereof, 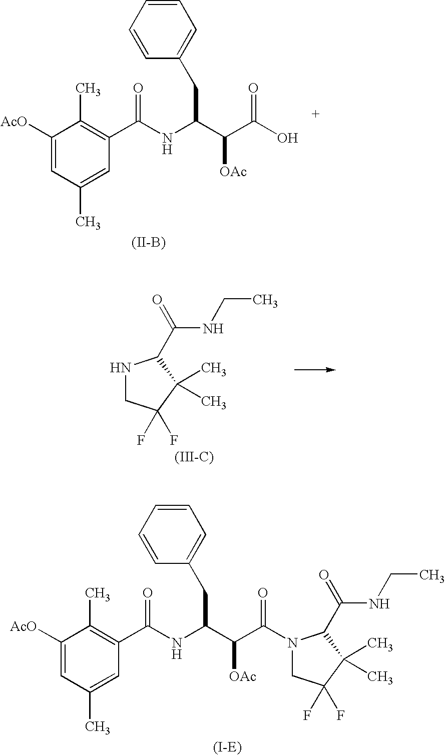
   to afford a compound of formula (I-E); and
(ii) deprotecting the compound of formula (I-E).

In still a further aspect of the present invention are provided methods of preparing compounds of formula (I-F), 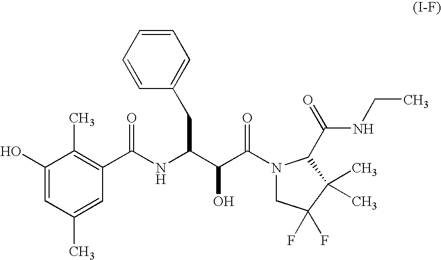
comprising:
(i) reacting a compound of formula (IV-A) with a compound of formula (V-B), 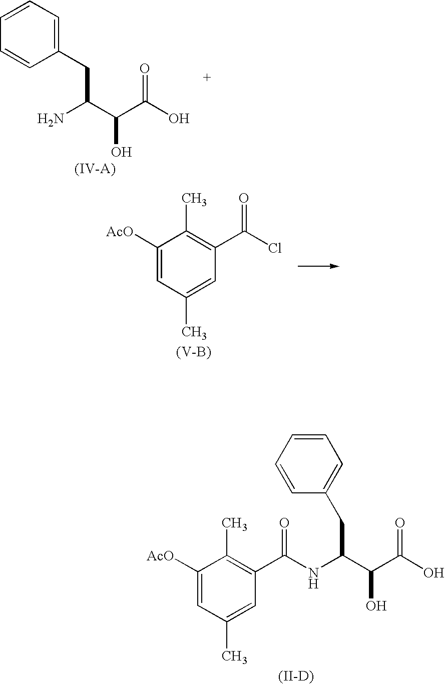
   to afford a compound of formula (II-D);
(ii) treating the compound of formula (II-D) with an acetylating agent, 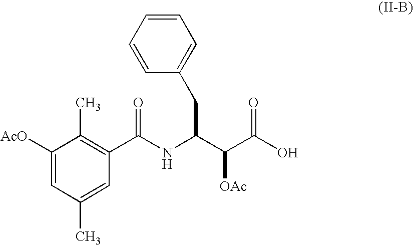
   to afford a compound of formula (II-B);
(iii) reacting the compound of formula (II-B) with a compound of formula (III-C), 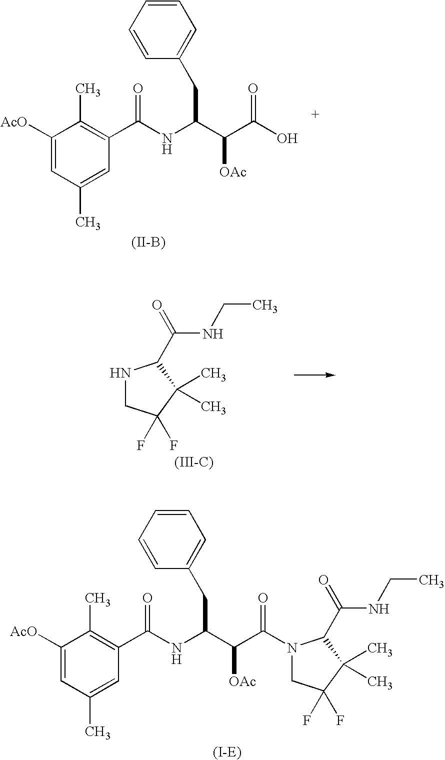
   to afford a compound of formula (I-E); and
(iv) deprotecting the compound of formula (I-E).

Another aspect of the present invention provides compounds of formula (I), or a salt or solvate thereof: 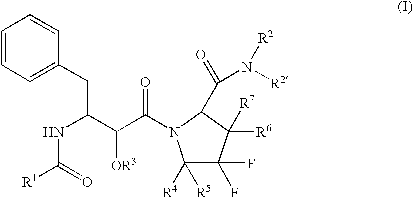
wherein:
R¹ is phenyl optionally substituted by at least one substituent independently chosen from C₁₋₆ alkyl, hydroxyl, C₁₋₆ alkylcarbonyloxy, C₆₋₁₀ arylcarbonyloxy, and heteroarylcarbonyloxy;
R² is C₂₋₆ alkenyl or C₁₋₆ alkyl optionally substituted with at least one halogen;
R^{2′} is H or C₁-C₄ alkyl;
R³ is a hydroxyl protecting group; and
R⁴, R⁵, R⁶ and R⁷ are independently chosen from H and C₁-C₆ alkyl.

In yet another aspect of the present invention are provided compounds of formula (I), wherein:
R¹ is phenyl optionally substituted by at least one substituent independently chosen from C₁₋₆ alkyl, hydroxyl, C₁₋₆ alkylcarbonyloxy, C₆₋₁₀ arylcarbonyloxy, and heteroarylcarbonyloxy;
R² is C₁₋₆ alkyl optionally substituted with at least one halogen;
R^{2′} is H or C₁-C₄ alkyl;
R³ is a hydroxyl protecting group; and
R⁴, R⁵, R⁶ and R⁷ are independently chosen from H and C₁-C₆ alkyl; or a salt or solvate thereof.

In still a further aspect of the present invention are provided compounds of formula (I), wherein:
R¹ is phenyl optionally substituted by at least one substituent independently chosen from C₁₋₆ alkyl, C₁₋₆ alkylcarbonyloxy, C₆₋₁₀ arylcarbonyloxy, and heteroarylcarbonyloxy;
R² is C₁₋₆ alkyl optionally substituted with at least one halogen;
R^{2′} is H or C₁-C₄ alkyl;
R³ is a hydroxyl protecting group; and
R⁴, R⁵, R⁶ and R⁷ are independently selected from H and C₁-C₆ alkyl; or a salt or solvate thereof.

The present invention also provides compounds of formula (I), wherein:
R¹ is phenyl substituted by at least one substituent independently chosen from methyl and methylcarbonyloxy;
R² is C₁₋₆ alkyl optionally substituted with at least one halogen;
R^{2′} is hydrogen;
R³ is a hydroxyl-protecting group;
R⁴ and R⁵ are hydrogen; and
R⁶ and R⁷ are independently selected from H and C₁-C₆ alkyl; or a salt or solvate thereof.

Also provided are compounds of formula (I), wherein:
R¹ is phenyl substituted by at least one substituent independently chosen from methyl and methylcarbonyloxy;
R² is C₁₋₆ alkyl optionally substituted with at least one fluorine;
R^{2′} is hydrogen;
R³ is a hydroxyl-protecting group;
R⁴ and R⁵ are hydrogen; and
R⁶ and R⁷ are C₁-C₆ alkyl; or
a salt or solvate thereof.

In addition, the present invention provides compounds of formula (I), wherein:
R¹ is phenyl substituted by at least one substituent independently chosen from methyl and methylcarbonyloxy;
R² is C₁₋₆ alkyl substituted with at least one fluorine;
R^{2′} is hydrogen;
R³ is a hydroxyl protecting group;
R⁴ and R⁵ are hydrogen; and
R⁶ and R⁷ are methyl; or
a salt or solvate thereof.

Also provided are compounds of formula (I), wherein:
R¹ is phenyl substituted by at least one substituent independently chosen from methyl and methylcarbonyloxy;
R² is —CH₂CF₃;
R^{2′} is hydrogen;
R³ is a hydroxyl protecting group;
R⁴ and R⁵ are hydrogen; and
R⁶ and R⁷ are methyl; or
a salt or solvate thereof.

In still a further aspect of the present invention are provided compounds of formula (I), wherein:
R¹ is phenyl substituted by at least one substituent independently chosen from methyl and methylcarbonyloxy;
R² is CH₂CH₃;
R^{2′} is hydrogen;
R³ is a hydroxyl-protecting group;
R⁴ and R⁵ are hydrogen; and
R⁶ and R⁷ are C₁-C₆ alkyl; or
a salt or solvate thereof.

Also provided are compounds of formula (I), wherein R³ is C₁₋₆alkylcarbonyl and compounds of formula (I) wherein R³ is methylcarbonyl, or a salt or solvate thereof.

In yet another aspect of the present invention are provided compounds of formula (II), 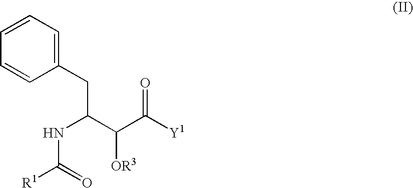
wherein:
R¹ is phenyl optionally substituted by at least one substituent independently chosen from C₁₋₆ alkyl, hydroxyl, C₁₋₆ alkylcarbonyloxy, C₆₋₁₀ arylcarbonyloxy, and heteroarylcarbonyloxy;
R³ is hydrogen or a hydroxyl protecting group; and
Y¹ is a leaving group or hydroxyl.

In another aspect of the present invention are provided compounds of formula (II), wherein:
R¹ is phenyl optionally substituted by at least one substituent independently chosen from C₁₋₄ alkyl, hydroxyl, C₁₋₆ alkylcarbonyloxy, C₆₋₁₀ arylcarbonyloxy, and heteroarylcarbonyloxy;
R³ is a hydroxyl protecting group; and
Y¹ is a leaving group or hydroxyl; or
a salt or solvate thereof.

In yet another aspect of the present invention are provided compounds of formula (II), wherein:
R¹ is phenyl optionally substituted by at least one substituent independently chosen from C₁₋₆ alkyl, hydroxyl, C₁₋₆ alkylcarbonyloxy, C₆₋₁₀ arylcarbonyloxy, and heteroarylcarbonyloxy;
R³ is a hydroxyl protecting group; and
Y¹ is hydroxyl; or
a salt or solvate thereof.

In still a further aspect of the present invention are provided compounds of formula (II), wherein:
R¹ is phenyl optionally substituted by at least one substituent independently chosen from methyl, hydroxyl, and C₁₋₆ alkylcarbonyloxy;
R³ is a hydroxyl protecting group; and
Y¹ is hydroxyl; or
a salt or solvate thereof.

The present invention also provides compounds of formula (II), wherein:
R¹ is phenyl optionally substituted by at least one substituent independently chosen from methyl, hydroxyl, and methylcarbonyloxy;
R³ is a hydroxyl protecting group; and
Y¹ is hydroxyl; or
a salt or solvate thereof.

Another aspect of the present invention also provides compounds of formula (II), wherein:
R¹ is phenyl substituted by methyl and methylcarbonyloxy;
R³ is a methylcarbonyl; and
Y¹ is hydroxyl; or
a salt or solvate thereof.

Another aspect of the present invention features compounds of formulae (I-C), (I-D), (I-E), (I-F), (II-A), (II-B), (III-B), and (III-C): 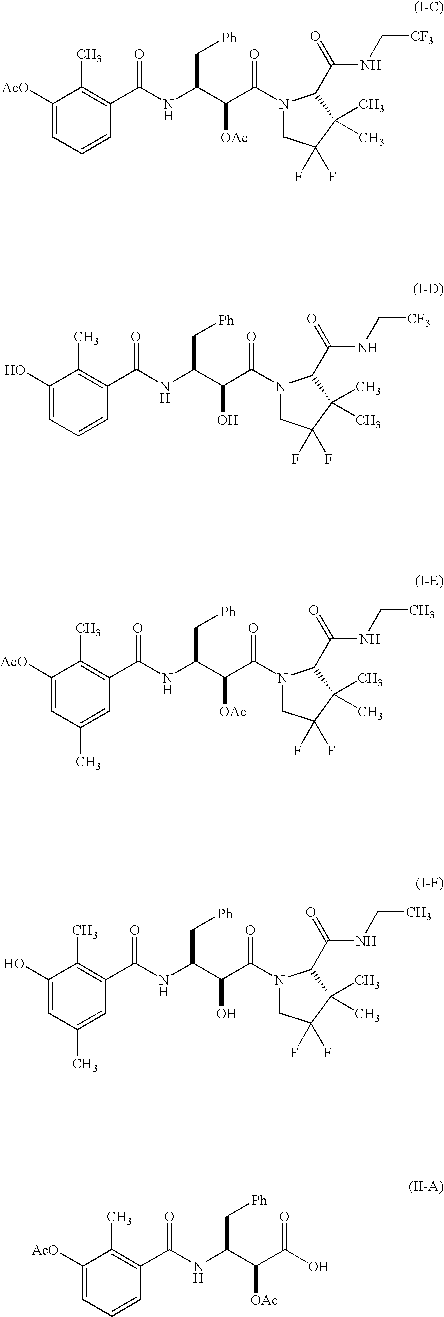 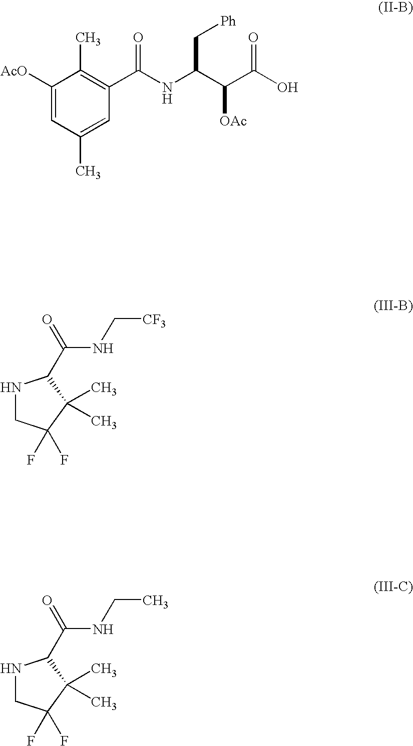
all of which are intermediates useful in the preparation of compounds of formula (I).

Another aspect of the present invention provides for the preparation of compounds of formula (II-A), 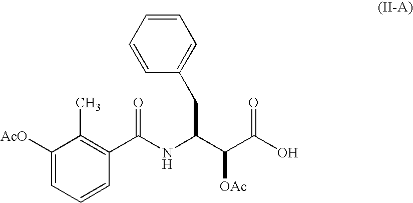
comprising:
treating a compound of formula (II-C) with an acetylating agent.

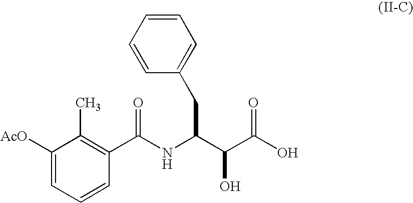

In another aspect of the present invention are provided methods of preparing compounds of formula (II-A) wherein the acetylating agent is acetic anhydride.

Another aspect of the present invention provides for the preparation of compounds of formula (II-B), 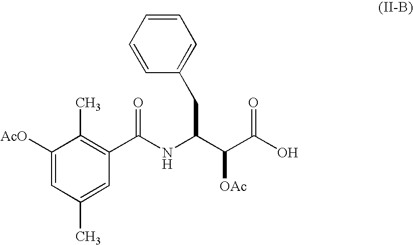
comprising:
treating a compound of formula (II-D) with an acetylating agent.

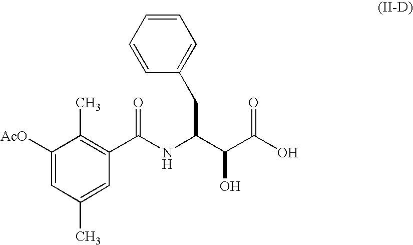

In another aspect of the present invention are provided methods of preparing compounds of formula (II-B) wherein the acetylating agent is acetic anhydride.

The present invention also concerns amorphous (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide, or a pharmaceutically acceptable salt or solvate thereof.

In still another aspect of the present invention is provided crystalline (2S)4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide (compound I-D), or a pharmaceutically acceptable salt or solvate thereof.

The invention also provides a crystal form of (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide, exhibiting a characteristic peak in the powder x-ray diffraction pattern, expressed in degrees two-theta, of about 8.7.

In another aspect, is provided a crystal form of (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide exhibiting characteristic peaks in the powder x-ray diffraction pattern, expressed in degrees two-theta, at about 8.7 and about 20.4. In yet another aspect, the crystal form exhibits characteristic peaks in the powder x-ray diffraction pattern, expressed in degrees two-theta, at about 8.7, about 20.4, and about 16.2. In still another aspect, the crystal form exhibits characteristic peaks in the powder x-ray diffraction pattern, expressed in degrees two-theta, at about 8.7, about 20.4, about 16.2, and about 11.7. In still another aspect, the crystal form exhibits characteristic peaks in the powder x-ray diffraction pattern, expressed in degrees two-theta, at about 8.7, about 20.4, about 16.2, about 11.7, and about 8.0.

Still another aspect of the present invention provides a crystal form of (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide exhibiting characteristic peaks in the powder x-ray diffraction pattern, expressed in degrees two-theta, in the range 8.6-8.8. A still further aspect provides a crystal form of (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide exhibiting characteristic peaks in the powder x-ray diffraction pattern, expressed in degrees two-theta, in the range 8.6-8.8 and in the range 20.3-20.5. In yet another aspect, the crystal form exhibits characteristic peaks in the powder x-ray diffraction pattern, expressed in degrees two-theta, in the range 8.6-8.8, in the range 20.3-20.5, and in the range 16.1-16.3. In still another aspect, the crystal form exhibits characteristic peaks in the powder x-ray diffraction pattern, expressed in degrees two-theta, in the range 8.6-8.8, in the range 20.3-20.5, in the range 16.1-16.3, and in the range 11.6-11.8. In still another aspect, the crystal form exhibits characteristic peaks in the powder x-ray diffraction pattern, expressed in degrees two-theta, in the range 8.6-8.8, in the range 20.3-20.5, in the range 16.1-16.3, in the range 11.6-11.8, and in the range 7.9-8.1.

The present invention further provides a crystalline form of (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide exhibiting peaks in the Raman scattering spectrum, expressed in Raman shift (wavenumbers, cm⁻¹), at about 1004; or at about 1004, and about 1079; or at about 1004, about 1079, and about 760; or at about 1004, about 1079, about 760, and about 838; or at about 1004, and about 1079, at about 1004, about 1079, and about 760; or at about 1004, about 1079, about 760, about 838, about 518, about 540, about 599, about 1475, and about 1715.

Also provided herein is a crystalline form of (2S)-4,4-difluoro-1-[(2S,3S)-2-hydroxy-3-(3-hydroxy-2-methyl-benzoylamino)₄-phenyl-butyryl]-3,3-dimethyl-pyrrolidine-2-carboxylic acid (2,2,2-trifluoroethyl)-amide exhibiting any combination of characteristic peaks in the powder X-ray diffraction pattern described above and any combination of the peaks in the Raman scattering spectrum described above. For example, the present invention affords a crystalline form of (2S)-4,4-difluoro-1-[(2S, 3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide exhibiting a characteristic peak in the powder x-ray diffraction pattern, expressed in degrees two-theta, in the range 8.6-8.8, and a peak in the Raman scattering spectrum, expressed in Raman shift (wavenumbers, cm⁻¹), at about 1004.

A still further aspect of the present invention provides a crystalline form of (2S)-4,4-difluoro-1-[(2S, 3S)-2-hydroxy-3-(3-hydroxy-2-methyl-benzoylamino)₄-phenyl-butyryl]-3,3-dimethyl-pyrrolidine-2-carboxylic acid (2,2,2-trifluoroethyl)-amide exhibiting a melting temperature of between about 191° C. and about 200° C.

In yet another aspect are afforded methods of preparing a crystalline form of (2S)-4,4-difluoro-1-[(2S, 3S)-2-hydroxy-3-(3-hydroxy-2-methyl-benzoylamino)-4-phenyl-butyryl]-3,3-dimethyl-pyrrolidine-2arboxylic acid (2,2,2-trifluoroethyl)-amide, comprising:
a) deprotecting the compound of formula (I-C), 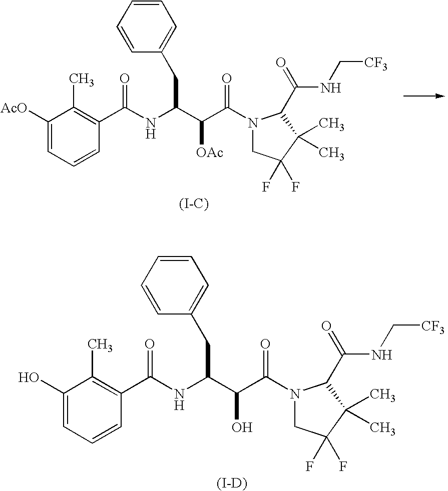
   to afford amorphous (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide (1-D); and
b) slurrying amorphous (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide in water to afford a crystalline form of (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide. In other aspects are provided such methods wherein the crystalline form of (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide exhibits characteristic peaks in the powder x-ray diffraction pattern, expressed in degrees two-theta, at about 8.7; or about 8.7 and about 20.4; or about 8.7, about 20.4, and about 16.2; or about 8.7, about 20.4, about 16.2, and about 11.7; or about 8.7, about 20.4, about 16.2, about 11.7, and about 8.0. In yet another aspect are provided such methods wherein the crystalline form of (2S)-4,4-difluoro-1-[(2S, 3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide exhibits characteristic peaks in the powder x-ray diffraction pattern, expressed in degrees two-theta, in the range 8.6-8.8. A still further aspect provides such methods wherein the crystal form of (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide exhibits characteristic peaks in the powder x-ray diffraction pattern, expressed in degrees two-theta, in the range 8.6-8.8 and in the range 20.3-20.5. In yet another aspect are provided such methods wherein the crystal form exhibits characteristic peaks in the powder x-ray diffraction pattern, expressed in degrees two-theta, in the range 8.6-8.8, in the range 20.3-20.5, and in the range 16.1-16.3. In still another aspect, are provided such methods wherein the crystal form exhibits characteristic peaks in the powder x-ray diffraction pattern, expressed in degrees two-theta, in the range 8.6-8.8, in the range 20.3-20.5, in the range 16.1-16.3, and in the range 11.6-11.8. In still another aspect, are provided such methods wherein the crystal form exhibits characteristic peaks in the powder x-ray diffraction pattern, expressed in degrees two-theta, in the range 8.6-8.8, in the range 20.3-20.5, in the range 16.1-16.3, in the range 11.6-11.8, and in the range 7.9-8.1. A still further aspect provides such methods wherein the crystal form of (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide exhibits peaks in the Raman scattering spectrum, expressed in Raman shift (wavenumbers, cm⁻¹), at about 1004; or at about 1004, and about 1079; or at about 1004, about 1079, and about 760; or at about 1004, about 1079, about 760, and about 838; or at about 1004, and about 1079, at about 1004, about 1079, and about 760; or at about 1004, about 1079, about 760, about 838, about 518, about 540, about 599, about 1475, and about 1715. Yet another aspect of the present invention provides such methods wherein the crystalline form of (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide exhibits a melting temperature of between about 191° C. and about 200° C.

Further provided are methods of preparing a crystalline form of (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide, comprising stirring amorphous (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide in the presence of water.

In still another aspect of the present invention are provided methods of preparing a crystalline form of (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide, comprising:
a) reacting a compound of formula (II-A) with a compound of formula (III-B), 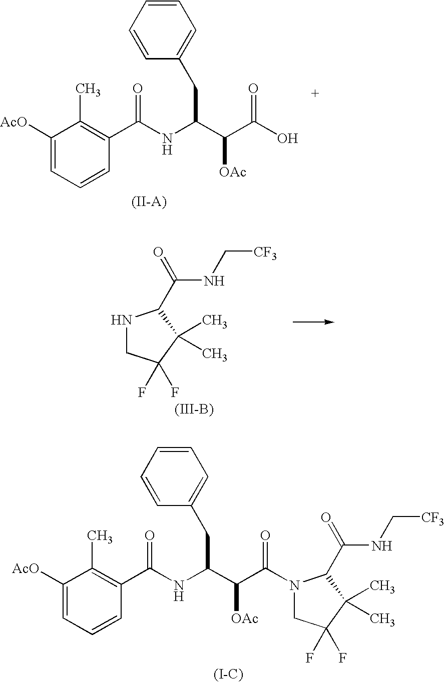
   to afford a compound of formula (I-C);
b) deprotecting the compound of formula (I-C), 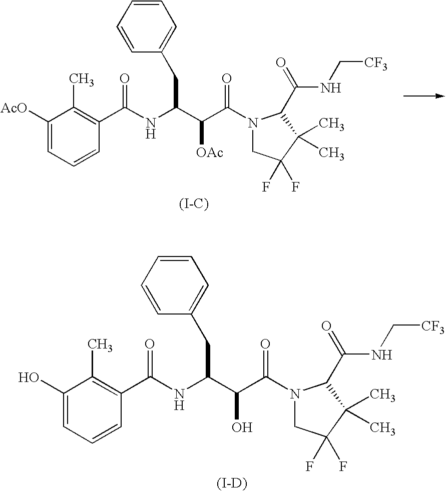
   to afford amorphous (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide (1-D); and
c) slurrying amorphous (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide in water to afford a crystalline form of (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide. In other aspects are provided such methods wherein the crystalline form of (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide exhibits characteristic peaks in the powder x-ray diffraction pattern, expressed in degrees two-theta, at about 8.7; or about 8.7 and about 20.4; or about 8.7, about 20.4, and about 16.2; or about 8.7, about 20.4, about 16.2, and about 11.7; or about 8.7, about 20.4, about 16.2, about 11.7, and about 8.0. In yet another aspect are provided such methods wherein the crystalline form of (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide exhibits characteristic peaks in the powder x-ray diffraction pattern, expressed in degrees two-theta, in the range 8.6-8.8. A still further aspect provides such methods wherein the crystal form of (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide exhibits characteristic peaks in the powder x-ray diffraction pattern, expressed in degrees two-theta, in the range 8.6-8.8 and in the range 20.3-20.5. In yet another aspect are provided such methods wherein the crystal form exhibits characteristic peaks in the powder x-ray diffraction pattern, expressed in degrees two-theta, in the range 8.6-8.8, in the range 20.3-20.5, and in the range 16.1-16.3. In still another aspect, are provided such methods wherein the crystal form exhibits characteristic peaks in the powder x-ray diffraction pattern, expressed in degrees two-theta, in the range 8.6-8.8, in the range 20.3-20.5, in the range 16.1-16.3, and in the range 11.6-11.8. In still another aspect, are provided such methods wherein the crystal form exhibits characteristic peaks in the powder x-ray diffraction pattern, expressed in degrees two-theta, in the range 8.6-8.8, in the range 20.3-20.5, in the range 16.1-16.3, in the range 11.6-11.8, and in the range 7.9-8.1. A still further aspect provides such methods wherein the crystal form of (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide exhibits peaks in the Raman scattering spectrum, expressed in Raman shift (wavenumbers, cm⁻¹), at about 1004; or at about 1004, and about 1079; or at about 1004, about 1079, and about 760, at about 1004, about 1079, about 760, and about 838; or at about 1004, and about 1079, at about 1004, about 1079, and about 760; or at about 1004, about 1079, about 760, about 838, about 518, about 540, about 599, about 1475, and about 1715. Yet another aspect of the present invention provides such methods wherein the crystalline form of (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide exhibits a melting temperature of between about 191° C. and about 200° C.

The present invention also concerns amorphous (2S)-4,4-difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide, or a pharmaceutically acceptable salt or solvate thereof.

The present invention also concerns crystalline (2S)-4,4-difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide (compound I-F), or a pharmaceutically acceptable salt or solvate thereof.

Another aspect of the present invention provides a crystalline form of (2S)-4,4-difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide exhibiting a characteristic peak in the powder x-ray diffraction pattern, expressed in degrees two-theta, at about 8.6.

A further aspect of the present invention provides a crystalline form of (2S)-4,4-difluoro-1-[(2S, 3S)-2-hydroxy-3-(3-hydroxy-2,5-dimethyl-benzoylamino)-4-phenyl-butyryl]-3,3-dimethyl-pyrrolidine-2-carboxylic acid ethylamide exhibiting characteristic peaks in the powder x-ray diffraction pattern, expressed in degrees two-theta, at about 8.2 and about 8.6.

In still a further aspect of the present invention is a crystalline form of (2S)-4,4-difluoro-1-[(2S, 3S)-2-hydroxy-3-(3-hydroxy-2,5-dimethyl-benzoylamino)-4-phenyl-butyryl]-3,3-dimethyl-pyrrolidine-2-carboxylic acid ethylamide exhibiting characteristic peaks in the powder x-ray diffraction pattern, expressed in degrees two-theta, at about 8.2, about 8.6, and about 11.1; or at about 8.2, about 8.6, about 11.1, and about 14.7; or at about 8.2, about 8.6, about 11.1, about 14.7, and about 15.5; or about 8.2, about 8.6, about 11.1, about 14.7, about 15.5, and about 16.4; or at about 8.2, about 8.6, about 11.1, about 14.7, about 15.5, about 16.4, and about 17.0; or at about 8.2, about 8.6, about 11.1, about 14.7, about 15.5, about 16.4, about 17.0, about 17.8, about 18.4, and about 20.7.

In yet another aspect of the present invention is a crystalline form of (2S)-4,4-difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide exhibiting characteristic peaks in the powder x-ray diffraction pattern, expressed in degrees two-theta, in the range 8.1-8.3. A further aspect provides such a crystal form that exhibits characteristic peaks in the powder x-ray diffraction pattern in the range 8.1-8.3, the range 8.5-8.7, and 11.0-11.2; or in the range 8.1-8.3, in the range 8.5-8.7, in the range 11.0-11.2, and in the range 14.6-14.8; or in the range 8.1-8.3, in the range 8.5-8.7, in the range 11.0-11.2, in the range 14.6-14.8, and in the range 15.4-15.6; or in the range 8.1-8.3, in the range 8.5-8.7, in the range 11.0-11.2, in the range 14.6-14.8, in the range 15.4-15.6, and in the range 16.3-16.5; or in the range 8.1-8.3, in the range 8.5-8.7, in the range 11.0-11.2, in the range 14.6-14.8, in the range 15.4-15.6, in the range 16.3-16.5, and in the range 16.9-17.1; or in the range 8.1-8.3, in the range 8.5-8.7, in the range 11.0-11.2, in the range 14.6-14.8, in the range 15.4-15.6, in the range 16.3-16.5, in the range 16.9-17.1, in the range 17.7-17.9, in the range 18.3-18.5, and in the range 20.6-20.8.

The present invention further provides a crystalline form of (2S)-4,4-difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide exhibiting peaks in the Raman scattering spectrum, expressed in Raman shift (wavenumbers, cm⁻¹), at about 1002; or at about 1002, and about 1471; or at about 1002, about 1471, and about 463; or at about 1002, about 1471, about 463, and about 1695; or at about 1002, about 1471, about 463, about 1695, about 555, about 622, about 655, about 753, about 781, about 899, about 976, about 1032, about 1320, and about 1536.

Also provided herein is a crystalline form of (2S)-4,4-difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide exhibiting any combination of characteristic peaks in the powder X-ray diffraction pattern described above and any combination of the characteristic peaks in the Raman scattering spectrum described above. For example, the present invention affords a crystalline form of (2S)-4,4-difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide exhibiting a characteristic peak in the powder x-ray diffraction pattern, expressed in degrees two-theta, in the range 8.1-8.3, and a peak in the Raman scattering spectrum, expressed in Raman shift (wavenumbers, cm⁻¹), at about 1002.

A still further aspect of the present invention provides a crystalline form of (2S)-4,4-difluoro-1-[(2S,3S)-2-hydroxy-3-(3-hydroxy-2,5-dimethyl-benzoylamino)-4-phenyl-butyryl]-3,3dimethyl-pyrrolidine-2-carboxylic acid ethylamide exhibiting a melting temperature of between about 206° C. and about 217° C.

Further provided are methods of preparing a crystalline form of (2S)-4,4-difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide, comprising stirring amorphous (2S)-4,4-difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide in the presence of water.

In yet another aspect are afforded methods of preparing a crystalline form of (2S)-4,4-difluoro-1-[(2S, 3S)-2-hydroxy-3-(3-hydroxy-2,5-dimethyl-benzoylamino)-4-phenyl-butyryl]-3,3-dimethyl-pyrrolidine-2-carboxylic acid ethylamide, comprising:
a) deprotecting the compound of formula (I-E), 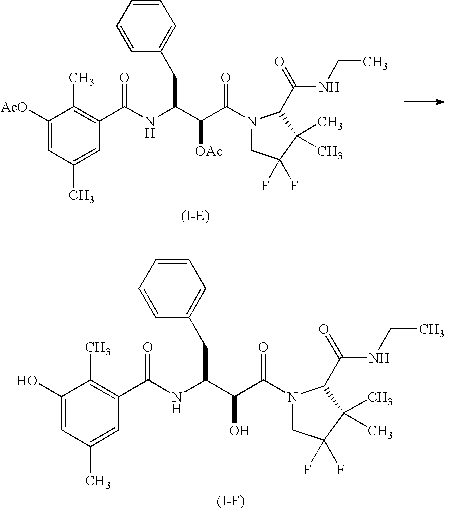
   to afford amorphous (2S)-4,4-difluoro-1-[(2S, 3S)-2-hydroxy-3-(3-hydroxy-2,5-dimethyl-benzoylamino)-4-phenyl-butyryl]-3,3-dimethyl-pyrrolidine-2-carboxylic acid ethylamide (I-F); and
b) slurrying amorphous (2S)-4,4-difluoro-1-[(2S,3S)-2-hydroxy-3-(3-hydroxy-2,5-dimethyl-benzoylamino)A-phenyl-butyryl]-3,3-dimethyl-pyrrolidine-2-carboxylic acid ethylamide in water to afford a crystalline form of (2S)-4,4-difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide. Further provided are such methods wherein the crystalline form of (2S)-4,4-difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide exhibits a characteristic peak in the powder x-ray diffraction pattern, expressed in degrees two-theta, at about 8.2. Also provided are such methods wherein the crystalline form of (2S)-4,4-difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide exhibits characteristic peaks in the powder x-ray diffraction pattern, expressed in degrees two-theta, at about 8.2, about 8.6, and about 11.1; or at about 8.2, about 8.6, about 11.1, and about 14.7; or at about 8.2, about 8.6, about 11.1, about 14.7, and about 15.5; or about 8.2, about 8.6, about 11.1, about 14.7, about 15.5, and about 16.4; or at about 8.2, about 8.6, about 11.1, about 14.7, about 15.5, about 16.4, and about 17.0; or at about 8.2, about 8.6, about 11.1, about 14.7, about 15.5, about 16.4, about 17.0, about 17.8, about 18.4, and about 20.7. In yet another aspect of the present invention are methods wherein the crystalline form of (2S)-4,4-difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide exhibiting characteristic peaks in the powder x-ray diffraction pattern, expressed in degrees two-theta, in the range 8.1-8.3. A further aspect provides such methods wherein the crystal form exhibits characteristic peaks in the powder x-ray diffraction pattern in the range 8.1-8.3, the range 8.5-8.7, and 11.0-11.2; or in the range 8.1-8.3, in the range 8.5-8.7, in the range 11.0-11.2, and in the range 14.6-14.8; or in the range 8.1-8.3, in the range 8.5-8.7, in the range 11.0-11.2, in the range 14.6-14.8, and in the range 15.4-15.6; or in the range 8.1-8.3, in the range 8.5-8.7, in the range 11.0-11.2, in the range 14.6-14.8, in the range 15.4-15.6, and in the range 16.3-16.5; or in the range 8.1-8.3, in the range 8.5-8.7, in the range 11.0-11.2, in the range 14.6-14.8, in the range 15.4-15.6, in the range 16.3-16.5, and in the range 16.9-17.1; or in the range 8.1-8.3, in the range 8.5-8.7, in the range 11.0-11.2, in the range 14.6-14.8, in the range 15.4-15.6, in the range 16.3-16.5, in the range 16.9-17.1, in the range 17.7-17.9, in the range 18.3-18.5, and in the range 20.6-20.8. In still another aspect are provided such methods wherein the crystalline form of (2S)-4,4-difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide exhibits peaks in the Raman scattering spectrum, expressed in Raman shift (wavenumbers, cm⁻¹), at about 1002; or at about 1002, and about 1471; or at about 1002, about 1471, and about 463; or at about 1002, about 1471, about 463, and about 1695; or at about 1002, about 1471, about 463, about 1695, about 555, about 622, about 655, about 753, about 781, about 899, about 976, about 1032, about 1320, and about 1536. Further provided herein are such methods wherein the crystalline form of (2S)-4,4-difluoro-1-[(2S, 3S)-2-hydroxy-3-(3-hydroxy-2,5-dimethyl-benzoylamino)-4-phenyl-butyryl]-3,3-dimethyl-pyrrolidine-2-carboxylic acid ethylamide exhibiting a melting temperature of between about 206° C. and about 217° C.

In still another aspect of the present invention are provided methods of preparing a crystalline form of (2S)-4,4-difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide, comprising:
a) reacting a compound of formula (II-B) with a compound of formula (III-C), 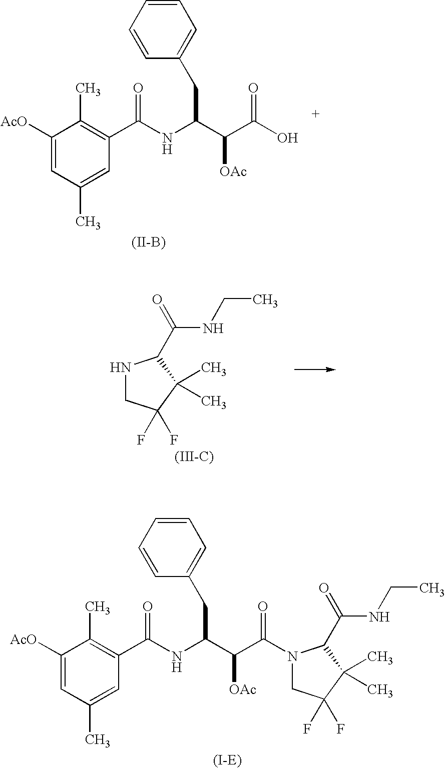
   to afford a compound of formula (I-E);
b) deprotecting the compound of formula (I-E), 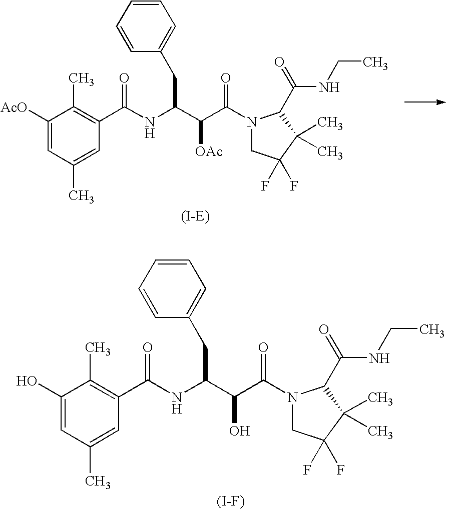
   to afford amorphous (2S)-4,4-difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide (I-F); and
c) slurrying amorphous (2S)-4,4-difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide in water to afford a crystalline form of (2S)-4,4-difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide. Further provided are such methods wherein the crystalline form of (2S)-4,4-difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide exhibits a characteristic peak in the powder x-ray diffraction pattern, expressed in degrees two-theta, of about 8.2. Also provided are such methods wherein the crystalline form of (2S)-4,4-difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide exhibits characteristic peaks in the powder x-ray diffraction pattern, expressed in degrees two-theta, at about 8.2, about 8.6, and about 11.1; or at about 8.2, about 8.6, about 11.1, and about 14.7; or at about 8.2, about 8.6, about 11.1, about 14.7, and about 15.5; or about 8.2, about 8.6, about 11.1, about 14.7, about 15.5, and about 16.4; or at about 8.2, about 8.6, about 11.1, about 14.7, about 15.5, about 16.4, and about 17.0; or at about 8.2, about 8.6, about 11.1, about 14.7, about 15.5, about 16.4, about 17.0, about 17.8, about 18.4, and about 20.7. In yet another aspect of the present invention are methods wherein the crystalline form of (2S)-4,4-difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide exhibiting characteristic peaks in the powder x-ray diffraction pattern, expressed in degrees two-theta, in the range 8.1-8.3. A further aspect provides such methods wherein the crystal form exhibits characteristic peaks in the powder x-ray diffraction pattern in the range 8.1-8.3, the range 8.5-8.7, and 11.0-11.2; or in the range 8.1-8.3, in the range 8.5-8.7, in the range 11.0-11.2, and in the range 14.6-14.8; or in the range 8.1-8.3, in the range 8.5-8.7, in the range 11.0-11.2, in the range 14.6-14.8, and in the range 15.4-15.6; or in the range 8.1-8.3, in the range 8.5-8.7, in the range 11.0-11.2, in the range 14.6-14.8, in the range 15.4-15.6, and in the range 16.3-16.5; or in the range 8.1-8.3, in the range 8.5-8.7, in the range 11.0-11.2, in the range 14.6-14.8, in the range 15.4-15.6, in the range 16.3-16.5, and in the range 16.9-17.1; or in the range 8.1-8.3, in the range 8.5-8.7, in the range 11.0-11.2, in the range 14.6-14.8, in the range 15.4-15.6, in the range 16.3-16.5, in the range 16.9-17.1, in the range 17.7-17.9, in the range 18.3-18.5, and in the range 20.6-20.8. In still another aspect are provided such methods wherein the crystalline form of (2S)-4,4-difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide exhibits peaks in the Raman scattering spectrum, expressed in Raman shift (wavenumbers, cm⁻¹), at about 1002; or at about 1002, and about 1471; or at about 1002, about 1471, and about 463; or at about 1002, about 1471, about 463, and about 1695; or at about 1002, about 1471, about 463, about 1695, about 555, about 622, about 655, about 753, about 781, about 899, about 976, about 1032, about 1320, and about 1536. Further provided herein are such methods wherein the crystalline form of (2S)-4,4-difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide exhibiting a melting temperature of between about 206° C. and about 217° C.

Also provided herein are any of the above-described methods of preparing a crystalline form of a compound of the invention wherein the slurry of the amorphous form of the compound with water is performed at a concentration of from about 1 mg to about 100 mg of compound per milliliter of water, or from about 1 mg to about 75 mg of compound per milliliter of water, or from about 5 mg to about 75 mg of compound per milliliter of water, or from about 10 mg to about 75 mg of compound per milliliter of water, or from about 15 mg to about 50 mg of compound per milliliter of water, or from about 25 mg to about 50 mg of compound per milliliter of water, or about 30 mg of compound per milliliter of water.

Further provided herein are any of the above-described methods of preparing a crystalline form of a compound of the invention wherein the slurry with water is held at a temperature of from about 25° C. to about 95° C.; or from about 25° C. to about 85° C.; or from about 30° C. to about 75° C.; or from about 45° C. to about 75° C.; or from about 50° C. to about 75° C.; or about 60° C.

Further provided herein are any of the above-described methods of preparing a crystalline form of a compound of the invention wherein the slurry with water is stirred for a time period of between about 6 hours and about 48 hours, or from about 6 hours to about 24 hours, or from about 12 hours to about 24 hours, or about 16 hours.

The term “reacting,” as used herein, refers to a chemical process or processes in which two or more reactants are allowed to come into contact with each other to effect a chemical change or transformation. For example, when reactant A and reactant B are allowed to come into contact with each other to afford a new chemical compound(s) C, A is said to have “reacted” with B to produce C.

The term “protecting,” as used herein, refers to a process in which a functional group in a chemical compound is selectively masked by a non-reactive functional group in order to allow a selective reaction(s) to occur elsewhere on said chemical compound. Such non-reactive functional groups are herein termed “protecting groups.” For example, the term “hydroxy]protecting group,” as used herein refers to those groups that are capable of selectively masking the reactivity of a hydroxyl (—OH) group. The term “suitable protecting group,” as used herein refers to those protecting groups that are useful in the preparation of the compounds of the present invention. Such groups are generally able to be selectively introduced and removed using mild reaction conditions that do not interfere with other portions of the subject compounds. Protecting groups that are suitable for use in the processes and methods of the present invention are known to those of ordinary skill in the art. The chemical properties of such protecting groups, methods for their introduction and their removal can be found, for example, in T. Greene and P. Wuts, Protective Groups in Organic Synthesis (3^{rd} ed.), John Wiley & Sons, NY (1999). The terms “deprotecting,” “deprotected,” or “deprotect,” as used herein, are meant to refer to the process of removing a protecting group from a compound.

The term “slurry,” as used herein, means a liquid containing suspended solids, or a suspension of dispersed particles in a liquid medium, that usually must be agitated to retain its consistency. In the present invention it is specifically contemplated that the compound or compounds comprising the dispersed particles in the slurry may be insoluble, slightly soluble, or somewhat soluble in the liquid comprising the other portion of the slurry. Furthermore, the dispersed particles comprising the slurry may be of any size that is consistent with the formation of a slurry. The amount of the compound or compounds comprising the dispersed solids, the amount of the liquid or mixture of liquids forming the liquid phase of the slurry, and the temperature of the liquid/dispersed solid mixture, required to form a useful slurry will depend on the at least the identity of the compound or compounds comprising the dispersed solids and the liquid or liquids comprising the liquid phase of the slurry. The identities and amounts of the dispersed solids, liquids, and the temperature of the mixture required to form a useful slurry according to the present invention are choices within the knowledge of those of ordinary skill in the art and can be determined without undue experimentation.

The term “slurrying,” as used herein, means the process of creating a slurry. Such slurries may be prepared by any method known to those of skill in the art. For example, they can be prepared by adding the compound or compounds comprising the dispersed solid to the liquid or mixture of liquids comprising the liquid phase, followed by agitation. Alternatively, such a slurry may be formed by adding the liquid or mixture of liquids comprising the liquid phase of the slurry to the compound or compounds comprising the dispersed solid, followed by agitation. Useful methods of agitation are known to those of ordinary skill in the art and include, but are not limited to, rapid stirring using mechanical means, such as a magnetic stir bar or a paddle, and sonication.

The term “leaving group,” as used herein refers to a chemical functional group that generally allows a nucleophilic substitution reaction to take place at the atom to which it is attached. For example, in acid chlorides of the formula Cl—C(O)R, wherein R is alkyl, aryl, or heterocyclic, the —Cl group is generally referred to as a leaving group because it allows nucleophilic substitution reactions to take place at the carbonyl carbon. Suitable leaving groups are known to those of ordinary skill in the art and can include halides, aromatic heterocycles, cyano, amino groups (generally under acidic conditions), ammonium groups, alkoxide groups, carbonate groups, formates, and hydroxy groups that have been activated by reaction with compounds such as carbodiimides. For example, suitable leaving groups can include, but are not limited to, chloride, bromide, iodide, cyano, imidazole, and hydroxy groups that have been allowed to react with a carbodiimide such as dicyclohexylcarbodiimide (optionally in the presence of an additive such as hydroxybenzotriazole) or a carbodiimide derivative.

The term “acetylating agent,” as used herein refers to chemical compounds that are useful for the introduction of an acetyl group, —C(O)CH₃, onto a hydroxyl group in the compounds of the invention. The symbol “Ac-,” as used in chemical structures herein, is meant to represent an acyl group in the compounds of the invention. Useful acetylating agents include, but are not limited to, acetic anhydride, acetyl chloride, acetyl bromide, and acetyl iodide. In addition, such acetylating agents can be prepared in situ by reaction of an appropriate combination of compounds, such as the reaction of acetyl chloride with sodium iodide in acetone to afford an intermediate acetyl iodide agent. The term “acetic anhydride,” as used herein is meant to represent a compound with the chemical formula CH₃C(O)OC(O)CH₃.

As used herein, the term “aliphatic” represents a saturated or unsaturated, straight- or branched-chain hydrocarbon, containing 1 to 10 carbon atoms which may be unsubstituted or substituted by one or more of the substituents described below. The term “aliphatic” is intended to encompass alkyl, alkenyl and alkynyl groups.

As used herein, the term “C₁₋₆alkyl” represents a straight- or branched-chain saturated hydrocarbon, containing 1 to 6 carbon atoms that may be unsubstituted or substituted by one or more of the substituents described below. Exemplary alkyl substituents include, but are not limited to methyl (Me), ethyl (Et), propyl, isopropyl, butyl, isobutyl, t-butyl, and the like.

The term “alkenyl” represents a straight- or branched-chain hydrocarbon, containing one or more carbon-carbon double bonds and having 2 to 10 carbon atoms which may be unsubstituted or substituted by one or more of the substituents described below. Exemplary alkenyl substituents include, but are not limited to ethenyl, propenyl, butenyl, allyl, pentenyl and the like.

The term “phenyl,” as used herein refers to a fully unsaturated 6-membered carbocyclic group. A “phenyl” group may also be referred to herein as a benzene derivative.

The term “heteroaryl,” as used herein refers to a group comprising an aromatic monovalent monocyclic, bicyclic, or tricyclic group, containing 5 to 18 ring atoms, including 1 to 5 heteroatoms selected from nitrogen, oxygen and sulfur, which may be unsubstituted or substituted by one or more of the substituents described below. As used herein, the term “heteroaryl” is also intended to encompass the N-oxide derivative (or N-oxide derivatives, if the heteroaryl group contains more than one nitrogen such that more than one N-oxide derivative may be formed) of the nitrogen-containing heteroaryl groups described herein. Illustrative examples of heteroaryl groups include, but are not limited to, thienyl, pyrrolyl, imidazolyl, pyrazolyl, furyl, isothiazolyl, furazanyl, isoxazolyl, thiazolyl, pyridyl, pyrazinyl, pyrimidinyl, pyridazinyl, triazinyl, benzo[b]thienyl, naphtho[2,3-b]thianthrenyl, isobenzofuranyl, chromenyl, xanthenyl, phenoxathienyl, indolizinyl, isoindolyl, indolyl, indazolyl, purinyl, isoquinolyl, quinolyl, phthalazinyl, naphthyrdinyl, quinoxyalinyl, quinzolinyl, benzothiazolyl, benzimidazolyl, tetrahydroquinolinyl, cinnolinyl, pteridinyl, carbazolyl, beta-carbolinyl, phenanthridinyl, acridinyl, perimidinyl, phenanthrolinyl, phenazinyl, isothiazolyl, phenothiazinyl, and phenoxazinyl. Illustrative examples of N-oxide derivatives of heteroaryl groups include, but are not limited to, pyridyl N-oxide, pyrazinyl N-oxide, pyrimidinyl N-oxide, pyridazinyl N-oxide, triazinyl N-oxide, isoquinolyl N-oxide, and quinolyl N-oxide. Further examples of heteroaryl groups include the following moieties: 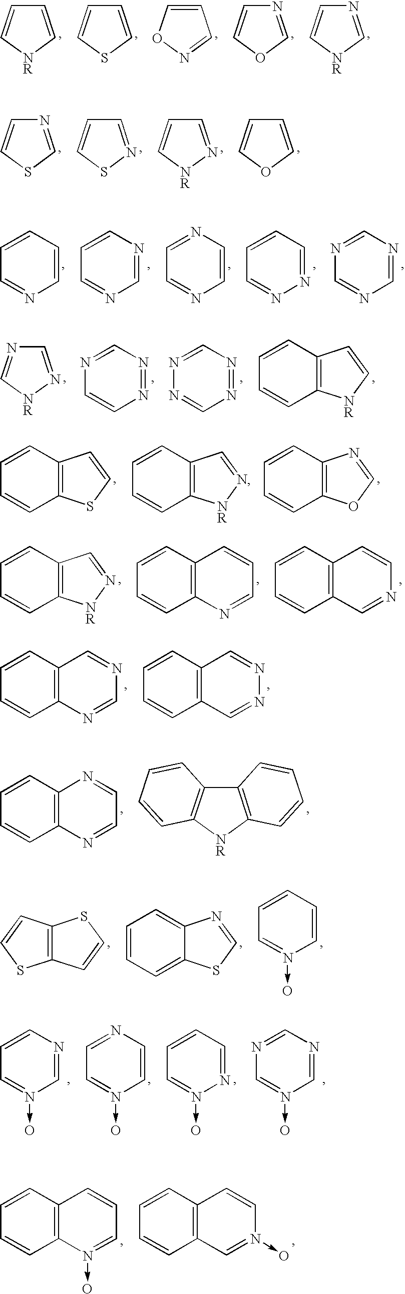 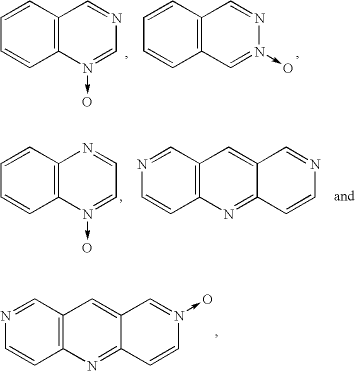
wherein R is H, alkyl, hydroxyl or represents a compound according to Formula I.

The terms “halogen” and “halo” represent chloro, fluoro, bromo or iodo substituents.

The term “C₁₋₆ alkylcarbonyloxy,” as used herein, refers to groups of the formula —OC(O)R, wherein R is an alkyl group comprising from 1 to 6 carbon atoms.

The term “C₆₋₁₀ arylcarbonyloxy,” as used herein, refers to a group of the formula —OC(O)R, wherein R is an aryl group comprising from 6 to 10 carbons.

The term “heteroarylcarbonyloxy,” as used herein, refers to a group of the formula —OC(O)R, wherein R is a heteroaromatic group as defined above.

The term “(2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide,” as used herein, refers to a compound that is also named “4,4-difluoro-1{(2S,3S)-2-hydroxy-3-[(3-hydroxy-2-methylbenzoyl)amino]-4-phenylbutanoyl}-3,3-dimethyl-N-(2,2,2-trifluoroethyl)-L-prolinamide, or “2-pyrrolidinecarboxamide, 4,4-difluoro-1-[(2S, 3S)-2-hydroxy-3-[(3-hydroxy-2-methylbenzoyl)amino]-1-oxo-4-phenylbutyl]-3,3-dimethyl-N-(2,2,2-trifluoroethyl)-, (2S),” and is represented by chemical formula (I-D).

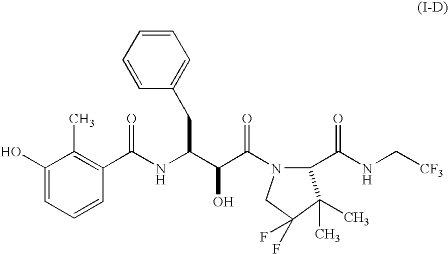

The term “(2S)-4,4-difluoro-1-[(2S, 3S)-2-hydroxy-3-(3-hydroxy-2,5-dimethyl-benzoylamino)-4-phenyl-butyryl]-3,3-dimethyl-pyrrolidine-2-carboxylic acid ethylamide,” as used herein, refers to a compound that is also named “2-pyrrolidinecarboxamide, N-ethyl-4,4-difluoro-1-[(2S,3S)-2-hydroxy-3-[(3-hydroxy-2,5-dimethylbenzoyl)amino]-1-oxo-4-phenylbutyl]-3,3-dimethyl-, (2S)-,” or “N-ethyl-4,4-difluoro-1{(2S,3S)-2-hydroxy-3-[(3-hydroxy-2,5-dimethylbenzoyl)amino]-4-phenylbutanoyl}-3,3-dimethyl-L-prolinamide,” and is represented by chemical formula (I-F).

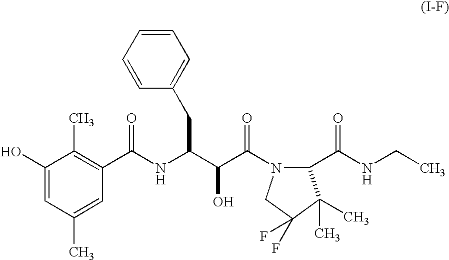

The term “crystalline,” as used herein, means the compound exhibits long-range order in three dimensions.

The term “amorphous,” as used herein is meant that the compound is not “crystalline.” Thus, the term amorphous is intended to include not only material which has essentially no order, but also material which may have some small degree of order, but the order is in less than three dimensions and/or is only over short distances. Amorphous material may be characterized by techniques known in the art such as powder x-ray diffraction (PXRD) crystallography, solid state NMR, or thermal techniques such as differential scanning calorimetry (DSC). It is specifically contemplated herein that “amorphous” materials referred to herein may comprise both amorphous and crystalline material. For example, a composition of the present invention may comprise the compound (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide, wherein 75% of the compound is an amorphous form and the remaining 25% is in a crystalline form. Such compositions herein are referred to as “amorphous.”

The compositions of the present invention may comprise both amorphous and crystalline (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide. In one embodiment, the composition comprises at least about 5% w/w of crystalline (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide of the total amount of (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide present. In other embodiments, the crystalline form is at least about 10% w/w, about 20% w/w, about 25% w/w, about 50% w/w, about 75% w/w, about 80% w/w, about 85% w/w, about 90% w/w, or at least about 95% w/w, of the total amount of (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide.

The compositions of the present invention may comprise both amorphous and crystalline (2S)-4,4-difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide. In one embodiment, the composition comprises at least about 5% w/w of crystalline (2S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide of the total amount of (2S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide present. In other embodiments, the crystalline form is at least about 10% w/w, about 20% w/w, about 25% w/w, about 50% w/w, about 75% w/w, about 80% w/w, about 85% w/w, about 90% w/w, or at least about 95% w/w, of the total amount of (2S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide present.

### BRIEF DESCRIPTION OF THE DRAWINGS

FIG. 1 is an X-ray diffraction pattern of a crystalline form of (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide.

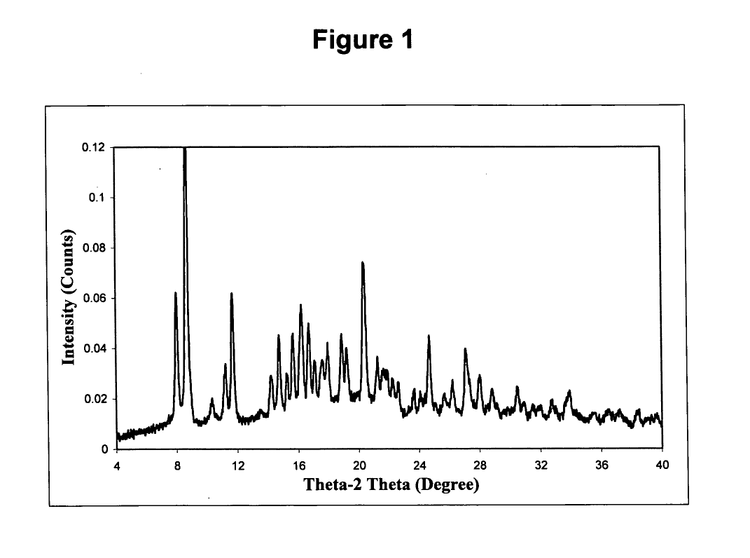

FIG. 2 is a characteristic differential Scanning Calorimetry Thermogram of a crystal form of (2S)-4,4-difluoro-1-[(2S, 3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide. Scan rates: 10° C. per minute. Vertical axis: Heat flow (w/g); Horizontal axis: Temperature (° C.)

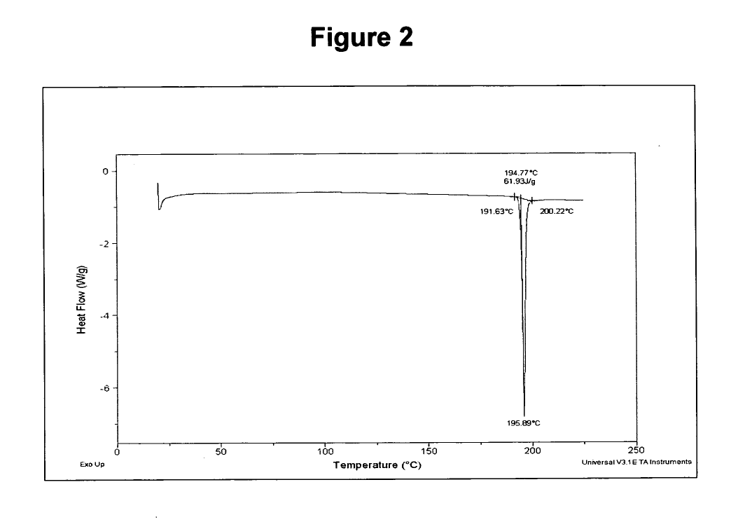

FIG. 3 is an X-ray diffraction pattern of a crystalline form of (2S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide.

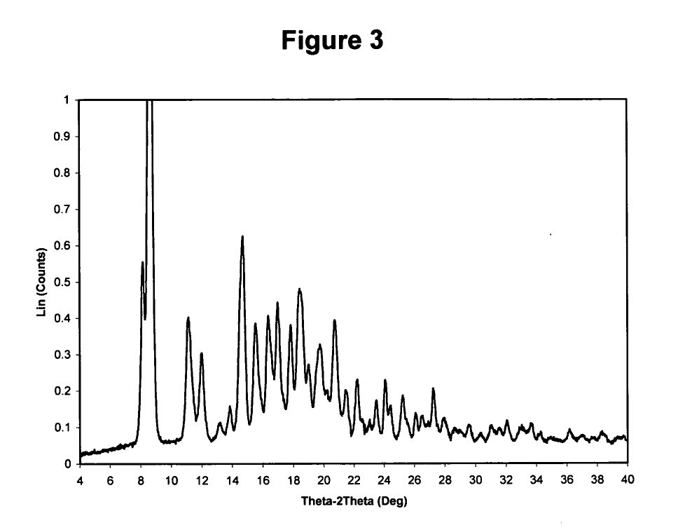

FIG. 4 is a characteristic differential Scanning Calorimetry Thermogram of a crystal form of (2S)-4,4-Difluoro-1-[(2S, 3S)-2-hydroxy-3-(3-hydroxy-2,5-dimethyl-benzoylamino)-4-phenyl-butyryl]-3,3-dimethyl-pyrrolidine-2-carboxylic acid ethylamide. Scan rates: 10° C. per minute. Vertical axis: Heat flow (w/g); Horizontal axis: Temperature (° C.)

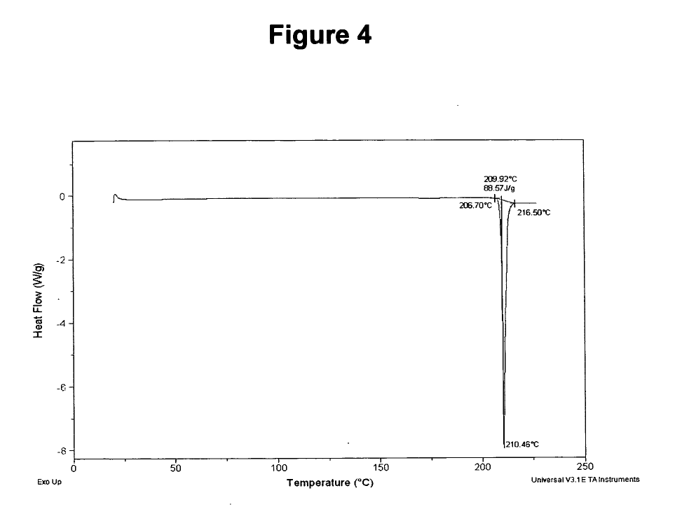

FIG. 5 is a characteristic Raman Scattering spectra of a crystalline form of (2S)-4,4-difluoro-1-[(2S, 3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide, measured at a resolution of 4 cm⁻¹.

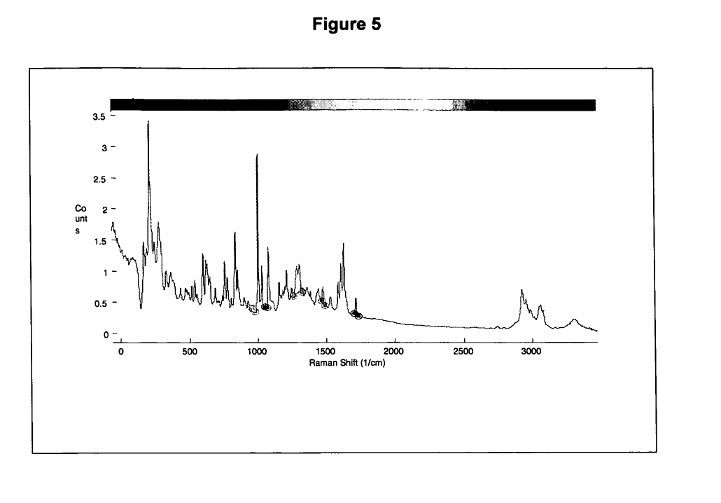

FIG. 6 is a characteristic Raman Scattering spectra of a crystalline form of (2S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide, measured at a resolution of 4 cm⁻¹.

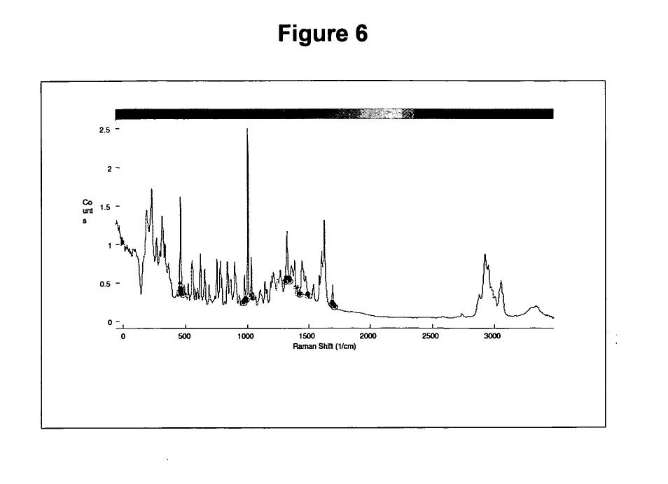

### DETAILED DESCRIPTION

In accordance with a convention used in the art, 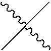
is used in structural formulas herein to depict the bond that is the point of attachment of the moiety or substituent to the core or backbone structure. When the phrase, “substituted with at least one substituent” is used herein, it is meant to indicate that the group in question may be substituted by at least one of the substituents chosen. The number of substituents a group in the compounds of the invention may have depends on the number of positions available for substitution. For example, an aryl ring in the compounds of the invention may contain from 1 to 5 additional substituents, depending on the degree of substitution present on the ring. Those of ordinary skill in the art can determine the maximum number of substituents that a group in the compounds of the invention may have.

The crystal forms comprising the present invention have been characterized using X-ray diffractometry. One of ordinary skill in the art will appreciate that an X-ray diffraction pattern may be obtained with a measurement error that is dependent upon the measurement conditions employed. In particular, it is generally known that intensities in an X-ray diffraction pattern may fluctuate depending upon measurement conditions employed. It should be further understood that relative intensities may also vary depending upon experimental conditions and, accordingly, the exact order of intensity should not be taken into account. Additionally, a measurement error of diffraction angle for a conventional X-ray diffraction pattern is typically about 0.1 expressed in degrees 2-theta, and such degree of measurement error should be taken into account as pertaining to the aforementioned diffraction angles. Consequently, it is to be understood that the crystal form of the present invention is not limited to the crystal form that provides an X-ray diffraction pattern completely identical to the X-ray diffraction pattern depicted in the accompanying Figures disclosed herein. Any crystal form that provides an X-ray diffraction pattern substantially identical to the one disclosed in the accompanying Figures falls within the scope of the present invention. The ability to ascertain substantial identities of X-ray diffraction patterns is within the purview of one of ordinary skill in the art.

If an inventive compound or an intermediate in the present invention is a base, a desired salt may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid or ethanesulfonic acid, or the like.

If an inventive compound or an intermediate in the present inventon is an acid, a desired salt may be prepared by any suitable method known to the art, including treatment of the free acid with an inorganic or organic base, such as an amine (primary, secondary, or tertiary); an alkali metal or alkaline earth metal hydroxide; or the like. Illustrative examples of suitable salts include organic salts derived from amino acids such as glycine and arginine; ammonia; primary, secondary, and tertiary amines; and cyclic amines, such as piperidine, morpholine, and piperazine; as well as inorganic salts derived from sodium, calcium, potassium, magnesium, manganese, iron, copper, zinc, aluminum, and lithium.

The compounds of the present invention contain at least one chiral center and may exist as single stereoisomers (e.g., single enantiomers or single diastereomers), any mixture of stereoisomers (e.g., any mixture of enantiomers or diastereomers) or racemic mixtures thereof. It is specifically contemplated that, unless otherwise indicated, all stereoisomers, mixtures and racemates of the present compounds are encompassed within the scope of the present invention. Compounds identified herein as single stereoisomers are meant to describe compounds that are present in a form that contains at least from at least about 90% to at least about 99% of a single stereoisomer of each chiral center present in the compounds. Where the stereochemistries of the chiral carbons present in the chemical structures illustrated herein are not specified, it is specifically contemplated that all possible stereoisomers are encompassed therein. The compounds of the present invention may be prepared and used in stereoisomerically pure form or substantially stereoisomerically pure form. As used herein, the term “stereoisomeric” purity refers to the “enantiomeric” purity and/or “diastereomeric” purity of a compound. The term “stereoisomerically pure form,” as used herein, is meant to encompass those compounds that contain from at least about 95% to at least about 99%, and all values in between, of a single stereoisomer. The term “substantially enantiomerically pure,” as used herein is meant to encompass those compounds that contain from at least about 90% to at least about 95%, and all values in between, of a single stereoisomer. The term “diastereomerically pure,” as used herein, is meant to encompass those compounds that contain from at least about 95% to at least about 99%, and all values in between, of a single diastereoisomer. The term “substantially diastereomerically pure,” as used herein, is meant to encompass those compounds that contain from at least about 90% to at least about 95%, and all values in between, of a single diastereoisomer. The terms “racemic” or “racemic mixture,” as used herein, refer to a mixture containing equal amounts of stereoisomeric compounds of opposite configuration. For example, a racemic mixture of a compound containing one stereoisomeric center would comprise equal amount of that compound in which the stereoisomeric center is of the (S)- and (R)-configurations. The term “enantiomerically enriched,” as used herein, is meant to refer to those compositions wherein one stereoisomer of a compound is present in a greater amount than the opposite stereoisomer. Similarly, the term “diastereomerically enriched,” as used herein, refers to those compositions wherein one diastereomer of compound is present in amount greater than the opposite diastereomer. The compounds of the present invention may be obtained in stereoisomerically pure (i.e., enantiomerically and/or diastereomerically pure) or substantially stereoisomerically pure (i.e., substantially enantiomerically and/or diastereomerically pure) form. Such compounds may be obtained synthetically, according to the procedures described herein using stereoisomerically pure or substantially stereoisomerically pure materials. Alternatively, these compounds may be obtained by resolution/separation of mixtures of stereoisomers, including racemic and diastereomeric mixtures, using procedures known to those of ordinary skill in the art. Exemplary methods that may be useful for the resolution/separation of stereoisomeric mixtures include derivitation with stereochemically pure reagents to form diastereomeric mixtures, chromatographic separation of diastereomeric mixtures, chromatographic separation of enantiomeric mixtures using chiral stationary phases, enzymatic resolution of covalent derivatives, and crystallization/re-rystallization. Other useful methods may be found in Enantiomers, Racemates, and Resolutions, J. Jacques, et al., 1981, John Wiley and Sons, New York, N.Y., the disclosure of which is incorporated herein by reference. Preferred stereoisomers of the compounds of this invention are described herein.

In one aspect of the present invention are provided compounds wherein the stereoisomeric centers (chiral carbons) have the following designated stereochemistry:

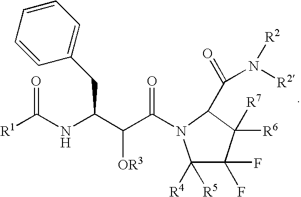

In still another aspect of the present invention are provided compounds wherein at least two of the stereoisomeric centers have the following stereochemistry:

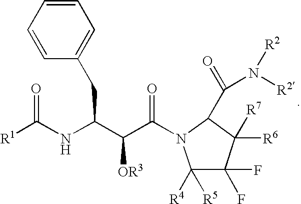

In yet another aspect of the present invention are provided compounds wherein three of the stereoisomeric centers have the following stereochemistry:

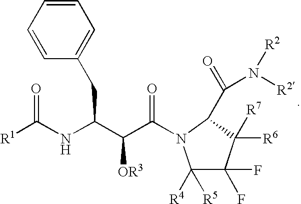

If the substituents themselves are not compatible with the synthetic methods of this invention, the substituent may be protected with a suitable protecting group that is stable to the reaction conditions used in these methods. The protecting group may be removed at a suitable point in the reaction sequence of the method to provide a desired intermediate or target compound. Suitable protecting groups and the methods for protecting and de-protecting different substituents using such suitable protecting groups are well known to those skilled in the art; examples of which may be found in T. Greene and P. Wuts, Protective Groups in Organic Synthesis (3^{rd} ed.), John Wiley & Sons, New York (1999), which is incorporated herein by reference in its entirety. In some instances, a substituent may be specifically selected to be reactive under the reaction conditions used in the methods of this invention. Under these circumstances, the reaction conditions convert the selected substituent into another substituent that is either useful in an intermediate compound in the methods of this invention or is a desired substituent in a target compound.

In the compounds of this invention, R² and R^{2′}, independently or taken together, may be a suitable nitrogen protecting group. As indicated above, suitable nitrogen protecting groups are known to those of ordinary skill in the art and any nitrogen-protecting group that is useful in the methods of preparing the compounds of this invention or may be useful in the HIV protease inhibitory compounds of this invention may be used. Exemplary nitrogen protecting groups include alkyl, substituted alkyl, carbamate, urea, amide, imide, enamine, sulfenyl, sulfonyl, nitro, nitroso, oxide, phosphinyl, phosphoryl, silyl, organometallic, borinic acid and boronic acid groups. Examples of each of these groups, methods for protecting nitrogen moieties using these groups and methods for removing these groups from nitrogen moieties are disclosed in T. Greene and P. Wuts, supra. Preferably, when R² and/or R^{2′} are independently suitable nitrogen protecting groups, suitable R² and R^{2′} substituents include, but are not limited to, carbamate protecting groups such as alkyloxycarbonyl (e.g., Boc: t-butyloxycarbonyl) and aryloxycarbonyl (e.g., Cbz: benzyloxycarbonyl, or FMOC: fluorene-9-methyloxycarbonyl), alkyloxycarbonyls (e.g., methyloxycarbonyl), alkyl or arylcarbonyl, substituted alkyl, especially arylalkyl (e.g., trityl (triphenylmethyl), benzyl and substituted benzyl), and the like. When R² and R^{2′} taken together are a suitable nitrogen protecting group, suitable R²/R^{2′} substituents include phthalimido and a stabase (1,2-bis (dialkylsilyl) ethylene).

The following processes illustrate the preparation of HIV protease inhibitors according to methods of the present invention. These compounds, prepared by the methods of the present invention, are potent inhibitors of HIV protease and thus are useful in the prevention and treatment of acquired immunodeficiency syndrome (AIDS) and AIDS related complex (“ARC”).

Unless otherwise indicated, variables according to the following processes are as defined above.

Starting materials, the synthesis of which are not specifically described herein or provided with reference to published references, are either commercially available or can be prepared using methods known to those of ordinary skill in the art. Certain synthetic modifications may be done according to methods familiar to those of ordinary skill in the art.

Compounds of formula (I), 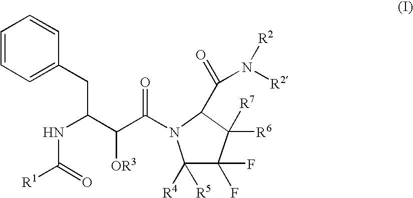
wherein R¹ is phenyl substituted by at least one hydroxyl group, and R², R^{2′}, R³, R⁴, R⁵, R⁶, R⁷, are as hereinbefore defined, may be prepared from compounds of formula I wherein R¹ is phenyl substituted by at least one group selected from C₁₋₆ alkylcarbonyloxy, C₆₋₁₀ arylcarbonyloxy, and heteroarylcarbonyloxy. The C₁₋₆ alkylcarbonyloxy, C₆₋₁₀ arylcarbonyloxy, and heteroarylcarbonyloxy groups may be cleaved under conditions that directly provide the desired hydroxyl substituted compounds of the invention. In general, the C₁₋₆ alkylcarbonyloxy, C₆₋₁₀ arylcarbonyloxy, and heteroarylcarbonyloxy groups may be cleaved under basic conditions, in a solvent that will not interfere with the desired transformation, and at a temperature that is compatible with the other reaction parameters, all of which are known to those of skill in the art. For example, appropriate bases include, but are not limited to, sodium bicarbonate, potassium bicarbonate, sodium carbonate, potassium carbonate, sodium hydroxide, potassium hydroxide, a sodium alkoxide such as sodium methoxide or sodium ethoxide, a potassium alkoxide such as potassium methoxide or potassium ethoxide, or a base formed in situ using an appropriate combination of reagents, such as a combination of a trialkyl or aryl amine in combination with an alkanol such as methanol. Or such a transformation may be accomplished using an acid that is known to those of skill in the art to be appropriate to cleave such a group without interfering with the desired transformation. Such acids include, but are not limited to, hydrogen halides such as hydrochloric acid or hydroiodic acid, an alkyl sulfonic acid such as methanesulfonic acid, an aryl sulfonic acid such as benzenesulfonic acid, nitric acid, sulfuric acid, perchloric acid, or chloric acid. Furthermore, appropriate solvents include those that are known to those of skill in the art to be compatible with the reaction conditions and include alkyl esters and aryl esters, alkyl, heterocyclic, and aryl ethers, hydrocarbons, alkyl and aryl alcohols, alkyl and aryl halogenated compounds, alkyl or aryl nitriles, alkyl and aryl ketones, and non-protic heterocyclic solvents. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Finally, these transformations may be conducted at temperatures from −20° C. to 100° C., depending on the specific reactants and solvents and is within the skill of one of ordinary skill in the art. Further suitable reaction conditions may be found in Greene, et al., Protective Groups in Organic Synthesis; John Wiley & Sons, New York, (1999).

Compounds of formula I wherein R³ is hydrogen and R¹, R², R^{2′}, R⁴, R⁵, R⁶, and R⁷, are as hereinbefore defined, may be prepared from compounds of formula (I) wherein R³ is a hydroxyl protecting group. The choice of a suitable hydroxy protecting group is within the knowledge of one of ordinary skill in the art. Suitable hydroxyl protecting groups that are useful in the present invention include, but are not limited to, alkyl or aryl esters, alkyl silanes, aryl silanes or alkylaryl silanes, alkyl or aryl carbonates, benzyl groups, substituted benzyl groups, ethers, or substituted ethers. The various hydroxy protecting groups can be suitably cleaved utilizing a number of reaction conditions known to those of ordinary skill in the art. The particular conditions used will depend on the particular protecting group as well as the other functional groups contained in the subject compound. Choice of suitable conditions is within the knowledge of those of ordinary skill in the art.

For example, if the hydroxy protecting group is an alkyl or aryl ester, cleavage of the protecting group may be accomplished using a suitable base, such as a carbonate, a bicarbonate, a hydroxide, an alkoxide, or a base formed in situ from an appropriate combination of agents. Furthermore, such reactions may be performed in a solvent that is compatible with the reaction conditions and will not interfere with the desired transformation. For example, suitable solvents may include alkyl esters, alkylaryl esters, aryl esters, alkyl ethers, aryl ethers, alkylaryl esters, cyclic ethers, hydrocarbons, alcohols, halogenated solvents, alkyl nitriles, aryl nitriles, alkyl ketones, aryl ketones, alkylaryl ketones, or non-protic heterocyclic compounds. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Finally, such reactions may be performed at an appropriate temperature from −20° C. to 100° C., depending on the specific reactants used. The choice of a suitable temperature is within the skill of one of ordinary skill in the art. Further suitable reaction conditions may be found in Greene, et al., Protective Grouts in Organic Synthesis, John Wiley & Sons, New York, (1999).

Additionally, if R³ is an alkyl silane, aryl silane or alkylaryl silane, such groups may be cleaved under conditions known to those of ordinary skill in the art. For example, such silane protecting groups may be cleaved by exposure of the subject compound to a source of fluoride ions, such as the use of an organic fluoride salt such as a tetraalkylammonium fluoride salt, or an inorganic fluoride salt. Suitable fluoride ion sources include, but are not limited to, tetramethylammonium fluoride, tetraethylammonium fluoride, tetrapropylammonium fluoride, tetrabutylammonium fluoride, sodium fluoride, and potassium fluoride. Alternatively, such silane protecting groups may be cleaved under acidic conditions using organic or mineral acids, with or without the use of a buffering agent. For example, suitable acids include, but are not limited to, hydrofluoric acid, hydrochloric acid, sulfuric acid, nitric acid, acetic acid, citric acid, and methanesulfonic acid. Such silane protecting groups may also be cleaved using appropriate Lewis acids. For example, suitable Lewis acids include, but are not limited to, dimethylbromo borane, triphenylmethyl tetrafluoroborate, and certain Pd (II) salts. Such silane protecting groups can also be cleaved under basic conditions that employ appropriate organic or inorganic basic compounds. For example, such basic compounds include, but are not limited to, sodium carbonate, potassium carbonate, sodium bicarbonate, potassium bicarbonate, sodium hydroxide, and potassium hydroxide. The cleavage of a silane-protecting group may be conducted in an appropriate solvent that is compatible with the specific reaction conditions chosen and will not interfere with the desired transformation. Among such suitable solvents are, for example, alkyl esters, alkylaryl esters, aryl esters, alkyl ethers, aryl ethers, alkylaryl esters, cyclic ethers, hydrocarbons, alcohols, halogenated solvents, alkyl nitriles, aryl nitriles, alkyl ketones, aryl ketones, alkylaryl ketones, or non-protic heterocyclic compounds. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Finally, such reactions may be performed at an appropriate temperature from −20° C. to 100° C., depending on the specific reactants used. The choice of a suitable temperature is within the skill of one of ordinary skill in the art. Further suitable reaction conditions may be found in Greene, et al., Protective Groups in Organic Synthesis, John Wiley & Sons, New York, (1999).

When R³ is a benzyl or substituted benzyl ether, cleavage of the protecting group may be accomplished by treating the subject compound with hydrogen in the presence of a suitable catalyst, oxidation with suitable compounds, exposure to light of particular wavelengths, electrolysis, treatment with protic acids, or treatment with Lewis acids. The choice of particular reagents to effect such a transformation will depend on the specific subject compound used and is within the skill of one of ordinary skill in the art. For example, such benzyl or substituted benzyl ethers may be cleaved using hydrogen gas in the presence of an appropriate catalyst. Suitable catalysts include, but are not limited to, 5% palladium on carbon, 10% palladium on carbon, 5% platinum on carbon, or 10% platinum on carbon. The choice of a particular catalyst and the amounts of catalyst, the amount of hydrogen gas, and the hydrogen gas pressure used to effect the desired transformation will depend upon the specific subject compound and the particular reaction conditions utilized. Such choices are within the skill of one of ordinary skill in the art. Furthermore, such benzyl and substituted benzyl ethers may be cleaved under oxidative conditions in which a suitable amount of an oxidizer is used. Such suitable oxidizers include, but are not limited to, dichlorodicyanoquinone (DDQ), ceric ammonium nitrate (CAN), ruthenium oxide in combination with sodium periodate, iron (III) chloride, or ozone. Additionally, such ethers may be cleaved using an appropriate Lewis acid. Such suitable Lewis acids include, but are not limited to, dimethylbromo borane, triphenylmethyl tetrafluoroborate, sodium iodide in combination with trifluoroborane-etherate, trichloroborane, or tin (IV) chloride. The cleavage of a benzyl or substituted benzyl ether protecting group may be conducted in an appropriate solvent that is compatible with the specific reaction conditions chosen and will not interfere with the desired transformation. Among such suitable solvents are, for example, alkyl esters, alkylaryl esters, aryl esters, alkyl ethers, aryl ethers, alkylaryl esters, cyclic ethers, hydrocarbons, alcohols, halogenated solvents, alkyl nitriles, aryl nitriles, alkyl ketones, aryl ketones, alkylaryl ketones, or non-protic heterocyclic compounds. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Finally, such reactions may be performed at an appropriate temperature from −20° C. to 100° C., depending on the specific reactants used. The choice of a suitable temperature is within the skill of one of ordinary skill in the art. Further suitable reaction conditions may be found in Greene, et al., Protective Groups in Organic Synthesis, John Wiley & Sons, New York, (1999).

When R³ is a methyl ether, cleavage of the protecting group may be accomplished by treating the subject compound with organic or inorganic acids or Lewis acids. The choice of a particular reagent will depend upon the type of methyl ether present as well as the other reaction conditions. The choice of a suitable reagent for cleaving a methyl ether is within the skill of one of ordinary skill in the art. Examples of suitable reagents include, but are not limited to, hydrochloric acid, sulfuric acid, nitric acid, para-toluenesulfonic acid, or Lewis acids such as boron trifluoride etherate. These reactions may be conducted in solvents that are compatible with the specific reaction conditions chosen and will not interfere with the desired transformation. Among such suitable solvents are, for example, alkyl esters, alkylaryl esters, aryl esters, alkyl ethers, aryl ethers, alkylaryl esters, cyclic ethers, hydrocarbons, alcohols, halogenated solvents, alkyl nitriles, aryl nitriles, alkyl ketones, aryl ketones, alkylaryl ketones, or non-protic heterocyclic compounds. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Finally, such reactions may be performed at an appropriate temperature from −20° C. to 100° C., depending on the specific reactants used. The choice of a suitable temperature is within the skill of one of ordinary skill in the art. Further suitable reaction conditions may be found in Greene, et al., Protective Groups in Organic Synthesis, John Wiley & Sons, New York, (1999).

When R³ is a carbonate, cleavage of the protecting group may be accomplished by treating the subject compound with suitable basic compounds Such suitable basic compounds may include, but are not limited to, sodium carbonate, sodium bicarbonate, potassium carbonate, potassium bicarbonate, sodium hydroxide, or potassium hydroxide. The choice of a particular reagent will depend upon the type of carbonate present as well as the other reaction conditions. These reactions may be conducted in solvents that are compatible with the specific reaction conditions chosen and will not interfere with the desired transformation. Among such suitable solvents are, for example, alkyl esters, alkylaryl esters, aryl esters, alkyl ethers, aryl ethers, alkylaryl esters, cyclic ethers, hydrocarbons, alcohols, halogenated solvents, alkyl nitriles, aryl nitriles, alkyl ketones, aryl ketones, alkylaryl ketones, or non-protic heterocyclic compounds. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Finally, such reactions may be performed at an appropriate temperature from −20° C. to 100° C., depending on the specific reactants used. The choice of a suitable temperature is within the skill of one of ordinary skill in the art. Further suitable reaction conditions may be found in Greene, et al., Protective Groups in Organic Synthesis; John Wiley & Sons, New York, (1999).

Furthermore, compounds of formula (I) wherein R¹ is phenyl substituted by at least one hydroxy group, and R³ is hydrogen, may be prepared from compounds of formula (I) wherein R¹ is phenyl optionally substituted by at least one substituent independently chosen from C₁₋₆ alkylcarbonyloxy, C₆₋₁₀ arylcarbonyloxy, and heteroarylcarbonyloxy; and R³ is a hydroxyl-protecting group. In these compounds, the R¹ C₁₋₆ alkylcarbonyloxy, C₆₋₁₀ arylcarbonyloxy, and heteroarylcarbonyloxy group and the R³ hydroxyl protecting group may be removed using reactions conditions in which both groups are removed concomitantly or they may be removed in step-wise fashion. For example, when R¹ is phenyl substituted by alkylcarbonyloxy and R³ is an alkyl ester, both groups may be cleaved by reacting the subject compound with a base in an appropriate solvent and at an appropriate temperature. The choice of a suitable base, solvent, and temperature will depend on the particular subject compound and the particular protecting groups being utilized. These choices are within the skill of one of ordinary skill in the art.

Alternatively, in compounds of formula (I) wherein R¹ is phenyl substituted by at least one group selected from C₁₋₆ alkylcarbonyloxy, C₆₋₁₀ arylcarbonyloxy, and heteroarylcarbonyloxy, and R³ is a hydroxyl protecting group, the C₁₋₆ alkylcarbonyloxy, C₆₋₁₀ arylcarbonyloxy, and heteroarylcarbonyloxy group and the R³ hydroxyl protecting group may be cleaved in a stepwise manner to afford a compound of formula (I) wherein R¹ is phenyl substituted by hydroxy and R³ is hydrogen. The choice of the R³ hydroxyl protecting group and the conditions to affect its cleavage will depend upon the specific subject compound chosen and is within the knowledge of one of ordinary skill in the art. For example, in the compounds of formula (I) wherein R¹ is phenyl substituted by C₁₋₆ alkylcarbonyloxy and R³ is a silane protecting group, the R³ silane protecting group may be cleaved first by treatment of the subject compound with a fluoride source such as tetrabutylammonium fluoride in acetonitrile at room temperature, followed by cleavage of the C₁₋₆ alkylcarbonyloxy group in R¹ by treatment with a base such as potassium hydroxide in a mixture of methanol and acetonitrile at room temperature.

Compounds of formula I wherein Z, R¹, R², R^{2′}, R³, R⁴, R⁵, R⁶, and R⁷, are as hereinbefore defined may be prepared by reacting a compound of formula (II), wherein Y¹ is a leaving group and R¹ and R³ are as hereinbefore defined, 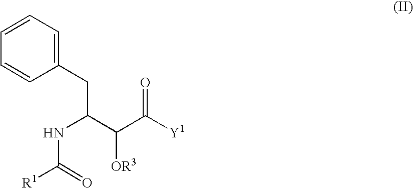
with a compound of formula (III), 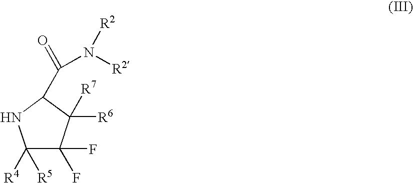
wherein R², R^{2′}, R⁴, R⁵, R⁶ and R⁷ are as hereinbefore defined, or a salt or solvate thereof, to afford a compound of formula (I).

In general, these reactions may be performed in a solvent that does not interfere with the reaction, for example alkyl or aryl ethers, alkyl or aryl esters, aromatic and aliphatic hydrocarbons, non-competitive alcohols, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C., depending on the specific reactants, solvents, and other optional additives used. Such reactions may also be promoted by the addition of optional additives. Examples of such additives include, but are not limited to, hydroxybenzotriazole (HOBt), hydroxyazabenzotriazole (HOAt), N-hydroxysuccinimide (HOSu), N-hydroxy-5-norbornene-endo-2,3-dicarboximide (HONB), 4-dimethylaminopyridine (DMAP). Whether these additives are necessary depends on the identity of the reactants, the solvent, and the temperature, and is within the skill of one of ordinary skill in the art.

In general, the leaving group Y¹ in the compounds of formula (II) should be such that it provides sufficient reactivity of the compounds of formula (II) with the compounds of formula (III). Compounds of formula (II) that contain such suitable leaving groups may be prepared, isolated and/or purified, and subsequently reacted with the compounds of formula (III). Alternatively, compounds of formula (II) with suitable leaving groups may be prepared and further reacted without isolation or further purification with the compounds of formula (III) to afford compounds of formula (I). Among suitable leaving groups, Y¹, are halides, aromatic heterocycles, sulfonic acid esters, phosphoric acid esters, anhydrides, or groups derived from the reaction of compounds of formula (II) wherein Y¹ is hydroxy with reagents such as carbodiimides or carbodiimide species. Examples of suitable leaving groups include, but are not limited to, chloride, iodide, imidazole, —OC(O)alkyl, —OC(O)aryl, —OC(O)Oalkyl, —OC(O)Oaryl, —OS(O₂)alkyl, —OS(O₂)aryl, —OPO(Oaryl)₂, OPO(Oalkyl)₂, and those derived from the reaction of the compounds of formula (II) wherein Y¹ is —OH with carbodiimides. Other suitable leaving groups are known to those of ordinary skill in the art and may be found, for example, in Humphrey, J. M.; Chamberlin, A. R. Chem. Rev. 1997, 97, 2243; Comprehensive Organic Synthesis; Trost, B. M., Ed.; Pergamon: New York, (1991); Vol. 6, pp 301-434; and Comprehensive Organic Transformations; Larock, R. C.; VCH: New York, (1989), Chapter 9.

Compounds of formula (II) where in Y¹ is a halogen can be prepared from compounds of formula II wherein Y¹ is hydroxy by reaction with a suitable agent. For example, the compounds of formula II wherein Y¹ is chloro may be prepared from compounds of formula (II) wherein Y¹ is hydroxy by reaction with agents such as thionyl chloride or oxalyl chloride. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (III) or they may be formed in situ and reacted with the compounds of formula (III) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

The present invention specifically contemplates that the compounds of formula (I) may be prepared by reacting compounds of formula (III) with compounds of formula (II), wherein R³ is hydrogen, an optionally substituted C₁₋₄ alkyl group, or a suitable protecting group, such as a C₁₋₆ alkylcarbonyl, C₆₋₁₀ arylcarbonyl, or heteroarylcarbonyl group.

Whether R³ in the compounds of formula (II) is hydrogen, an optionally substituted C₁₋₄ alkyl group, or a suitable protecting group is dependent on the specific product compounds desired and/or the specific reaction conditions used. Such choices are within the knowledge of one of ordinary skill in the art.

Compounds of formula (II) where in Y¹ is an aromatic heterocycle can be prepared from compounds of formula (II) wherein Y¹ is hydroxy by reaction with a suitable agent such as carbonyl diimidazole. These compounds may be isolated and then further reacted with the compounds of formula (III) or they may be formed in situ and reacted with the compounds of formula (III) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such knowledge is within the skill of one of ordinary skill in the art.

Compounds of formula (II) wherein Y¹ is OC(O)alkyl or —OC(O)aryl may be prepared from compounds of formula (II) wherein Y¹ is hydroxy by reaction with suitable reagents such acyl halides, acyl imidazoles, or carboxylic acid under dehydrating conditions. Suitable reagents may include, but are not limited to, acetyl chloride, acetyl iodide formed in situ from acetyl chloride and sodium iodide, acetyl imidazole, or acetic acid under dehydrating conditions. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula III or they may be formed in situ and reacted with the compounds of formula (III) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the skill of one of ordinary skill in the art.

Compounds of formula (II) wherein Y¹ is —OC(O)Oalkyl, —OC(O)Oaryl can be prepared from compounds of formula (II) wherein Y¹ is hydroxy by reaction with a suitable agents such as chloroformates of the formula Cl—C(O)Oalkyl or Cl—C(O)Oaryl. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (III) or they may be formed in situ and reacted with the compounds of formula (III) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the skill of one of ordinary skill in the art.

Compounds of formula (II) wherein Y¹ is —OS(O₂)alkyl or —OS(O₂)aryl can be prepared from compounds of formula (II) wherein Y¹ is hydroxy by reaction with a suitable agent such as an alkyl or aryl sulfonyl chloride. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (III) or they may be formed in situ and reacted with the compounds of formula (III) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the skill of one of ordinary skill in the art.

Alternatively, compounds of formula (I) may be prepared by reaction of compounds of formula (II), wherein Y¹ is —OH, with compounds of formula (III) under dehydrating conditions, utilizing agents such as carbodiimides or carbodiimide derived species. Such suitable agents include, but are not limited to, dicyclohexylcarbodiimide, diisopropylcarbodiimide, 1-[3-(dimethylamino)propyl]-3-ethylcarbodiimide hydrochloride (EDC), 2-chloro-4,6-dimethoxy-1,3,5-triazine (CDMT), cyanuric chloride, 4-(4,6-dimethoxy-1,3,5-triazin-2-yl)-4-methylmorpholinium chloride, O-(7-azabenzotriazol-1-yl)-N,N,N′,N′-tetramethyluronium hexafluorophosphate (HATU), carbonyldiimidazole (CDI), benzotriazole-1-yl-oxy-tris-(dimethylamino)-phosphoniumhexafluorophosphate (BOP), 2-ethoxy-1-ethoxycarbonyl-1,2-dihydroquinoline (EEDQ), 2-(1H-benzotriazole-1-yl)-1,1,3,3-tetramethyluronium hexafluorophosphate (HBTU), 2-(1H-benzotriazole-1-yl)-1,1,3,3-tetramethyluronium tetrafluoroborate (TBTU), and 3-(diethoxyphosphoryloxy)-1,2,3-benzotriazin-4(3H)-one (DEPBT). These reactions may be performed in the presence of optional additives. Suitable additives include, but are not limited to, hydroxybenzotriazole (HOBt), hydroxyazabenzotriazole (HOAt), N-hydroxysuccinimide (HOSu), N-hydroxy-5-norbornene-endo-2,3-dicarboximide (HONB), and 4-dimethylaminopyridine (DMAP). Whether these additives are necessary depends on the identity of the reactants, the solvent, and the temperature, and such choices are within the knowledge of one of ordinary skill in the art.

Compounds of formula (II), wherein R³ is a suitable protecting group and Y¹ and R¹ are as hereinbefore defined, may be prepared from compounds of formula (II) wherein R³ is hydrogen. The choice of a suitable protecting group is dependent upon the subject compound chosen and subsequent reaction conditions to which the compound of formula (II) will be subjected. Generally, R³ in the compounds of formula (II) can be chosen from alkyl or aryl esters, alkyl silanes, aryl silanes, alkylaryl silanes, carbonates, optionally substituted benzyl ethers, or other substituted ethers. Such protecting groups can be introduced into the compounds of formula (II) wherein R³ is hydrogen using methods known to those of ordinary skill in the art and as found in, for example, Greene, et al., Protective Groups in Organic Synthesis; John Wiley & Sons, New York, (1999). For example, as shown below, compound (5) was allowed to react with acetic anhydride in ethyl acetate and methanesulfonic acid at about 700C to afford compound (2).

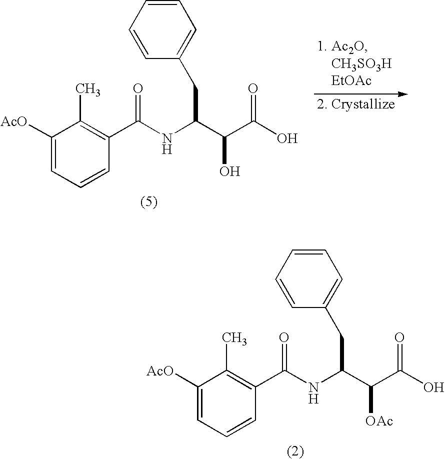

Compounds of formula (II), wherein Y¹ is hydroxy and R¹ and R³ are as hereinbefore defined, can be prepared by reaction of compounds of formula (IV), wherein Y¹ and R³ are as hereinbefore defined, with compounds of formula (V), wherein R¹ is as hereinbefore defined and Y² is hydroxy or a suitable leaving group, as shown below.

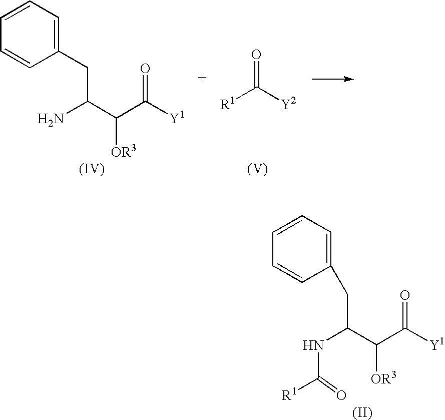

In general, these reactions may be performed in a solvent that does not interfere with the reaction, for example alkyl or aryl ethers, alkyl or aryl esters, aromatic and aliphatic hydrocarbons, non-competitive alcohols, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C., depending on the specific reactants, solvents, and other optional additives used. Such reactions may also be promoted by the addition of optional additives. Examples of such additives include, but are not limited to, hydroxybenzotriazole (HOBt), hydroxyazabenzotriazole (HOAt), N-hydroxysuccinimide (HOSu), N-hydroxy-5-norbornene-endo-2,3-dicarboximide (HONB), and 4-dimethylaminopyridine (DMAP). Whether these additives are necessary depends on the identity of the reactants, the solvent, and the temperature. Such choices are within the knowledge of one of ordinary skill in the art.

In general, the leaving group Y² in the compounds of formula (V) should be such that it provides sufficient reactivity with the amine in the compounds of formula (IV). Compounds of formula (V) that contain such suitable leaving groups may be prepared, isolated and/or purified, and subsequently reacted with the compounds of formula (IV). Alternatively, compounds of formula (V) with suitable leaving groups may be prepared and further reacted without isolation or further purification with the compounds of formula IV to afford compounds of formula (II). Among suitable leaving groups in the compounds of formula (V) are halides, aromatic heterocycles, sulfonic acid esters, phosphoric acid esters, anhydrides, or groups derived from the reaction of compounds of formula (V) wherein Y² is hydroxy with reagents such as carbodiimides or carbodiimide species. Examples of suitable leaving groups include, but are not limited to, chloride, iodide, imidazole, —OC(O)alkyl, —OC(O)aryl, —OC(O)Oalkyl, —OC(O)Oaryl, —OS(O₂)alkyl, —OS(O₂)aryl, —OPO(Oaryl)₂, OPO(Oalkyl)₂, and those derived from the reaction of the compounds of formula (V) wherein Y² is —OH with carbodiimides. Other suitable leaving groups are known to those of ordinary skill in the art and may be found, for example, in Humphrey, J. M.; Chamberlin, A. R. Chem. Rev., 1997, 97, 2243; Comprehensive Organic Synthesis; Trost, B. M., Ed.; Pergamon: New York, (1991); Vol. 6, pp 301-434; and Comprehensive Organic Transformations; Larock, R. C.; VCH: New York, (1989), Chapter 9.

Compounds of formula (V) where in Y² is a halogen can be prepared from compounds of formula (V) wherein Y² is hydroxy by reaction with a suitable agent. For example, the compounds of formula (V) wherein Y² is chloro may be prepared from compounds of formula (V) wherein Y² is hydroxy by reaction with agents such as thionyl chloride or oxalyl chloride. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (IV) or they may be formed in situ and reacted with the compounds of formula (IV) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art. For example, as shown below, compound (7) was allowed to react with compound (8) in a mixture of tetrahydrofuran and water, in the presence of triethylamine, at room temperature to afford the desired compound (5).

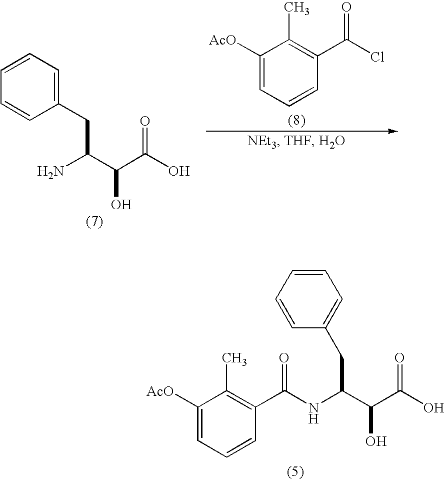

Compounds of formula (IV), wherein Y¹ is hydroxy and R³ is as defined above, are either commercially available or can be prepared by methods known to those of skill in the art.

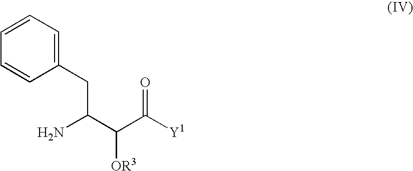

For example, the compounds of formula (IV) can be prepared as shown in the scheme below. In general, an N-protected amino acid derivative is reduced to an aldehyde using reducing agents that are suitable for such a transformation. For example, suitable reducing agents are dialkyl aluminum hydride agents, such as diisobutyl aluminum hydride for example. Another method of preparing the compounds of formula (IV) is to reduce an appropriate carboxylic acid to an alcohol with a suitable reducing agent such as LiAlH₄ or BH₃ or NaBH₄ for example, followed by oxidation of the alcohol to the corresponding aldehyde with PCC, under Swern conditions or using pyr.SO₃/DMSO/NEt₃ for example Another method of preparing the compounds of formula (IV) is to reduce an appropriate carboxylic acid derivative, such as a Weinreb amide or an acyl imidazole, using a suitable reducing agent such as LiAlH₄ or diisobutyl aluminum hydride for example. Alternatively, the compounds of formula (IV) can be prepared by the preparation of an appropriate aldehyde by reduction of the corresponding acid chloride. Next, a compound is added to the aldehyde that is the equivalent of adding a carboxylate CO₂ anion. For example, cyanide can be added to the aldehyde to afford a cyanohydrin that can then be hydrolyzed under either acidic or basic conditions to afford the desired compound, (d). Alternatively, nitromethane may be added to the aldehyde under basic conditions to afford an intermediate that is then converted into the desired compound. These compounds can be prepared according to the following procedures. In those compounds where Y³ is —CN, R. Pedrosa, et al., Tetrahedron Asymm. 2001, 12, 347. For those compounds in which Y³ is —CH₂NO₂, M. Shibasaki, et al., Tetrahedron Lett. 1994, 35, 6123.

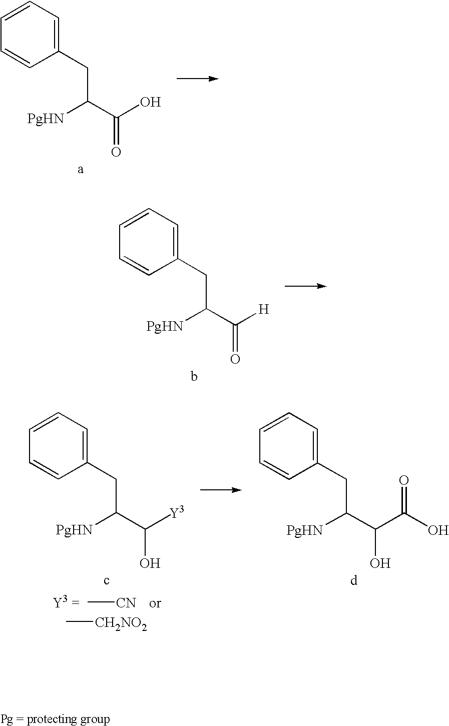

Compounds of formula (V), wherein Y² is hydroxy and R¹ is as hereinbefore defined, are either commercially available or can be prepared by methods known to those of skill in the art. For example, such compounds can be prepared from the corresponding alcohols by oxidation with suitable reagents. Such oxidation agents include, but are not limited to, KMnO₄, pyridinium dichromate (PDC), H₂Cr₂O₇ (Jones' reagent), and 2,2,6,6-tetramethylpiperidinyl-2-oxyl (TEMPO)/NaClO₂.

The compounds of formula (III), wherein R⁴ and R⁵ are hydrogen, R⁶, and R⁷ are methyl, and R² and R^{2′} are as hereinbefore defined, can be prepared according to the scheme below. The racemic material can be resolved according to methods known to those skilled in the art to provide compounds of formula (III) with an enantiomeric excess in the range of from 95% to 100%

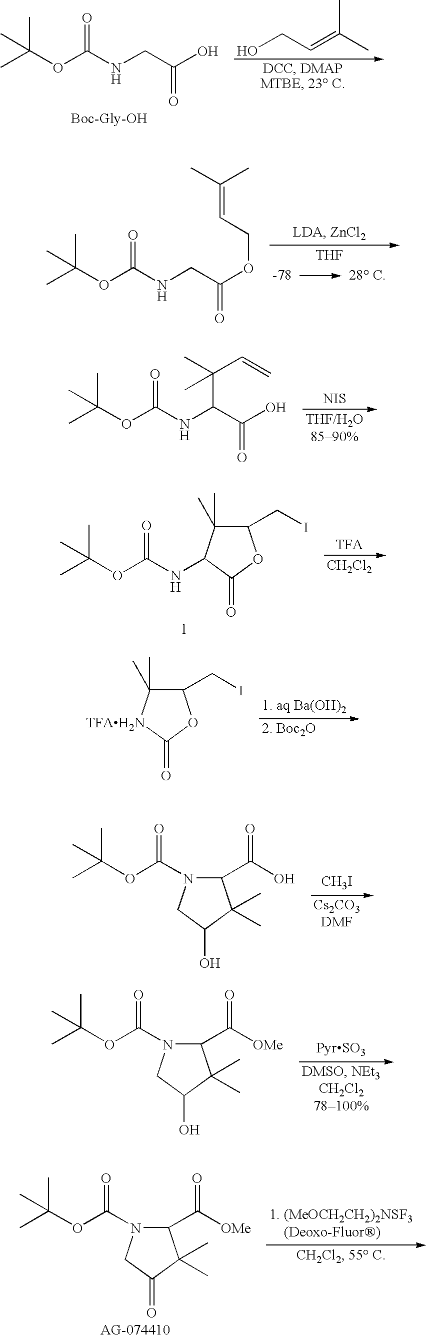

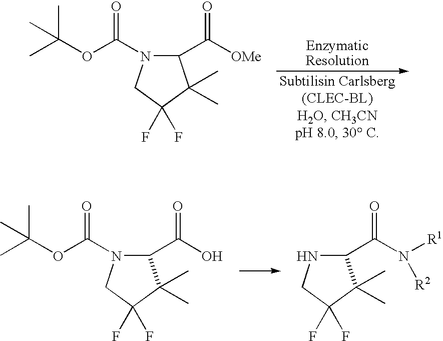

Alternatively, the compounds of formula (I), wherein R¹ is phenyl optionally substituted by at least one substituent independently chosen from C₁₋₆ alkyl, hydroxyl, C₁₋₆ alkylcarbonyloxy, C₆₋₁₀ arylcarbonyloxy, and heteroarylcarbonyloxy, and R², R^{2′}, R³, R⁴, R⁵, R⁶, and R⁷ are as hereinbefore defined, may be prepared by reaction of compounds of formula (VI), 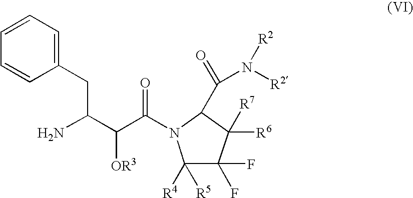
wherein R², R^{2′}, R³, R⁴, R⁵, R⁶, and R⁷ are as hereinbefore defined with compounds of formula (V), wherein R¹ and Y² are as hereinbefore defined.

In general, these reactions may be performed in a solvent that does not interfere with the reaction, for example alkyl or aryl ethers, alkyl or aryl esters, aromatic and aliphatic hydrocarbons, non-competitive alcohols, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrle, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C., depending on the specific reactants, solvents, and other optional additives used. Such reactions may also be promoted by the addition of optional additives. Examples of such additives include, but are not limited to, hydroxybenzotriazole (HOBt), hydroxyazabenzotriazole (HOAt), N-hydroxysuccinimide (HOSu), N-hydroxy-5-norbornene-endo-2,3-dicarboximide (HONB), and 4-dimethylaminopyridine (DMAP). Whether these additives are necessary depends on the identity of the reactants, the solvent, and the temperature. Such choices are within the knowledge of one of ordinary skill in the art.

In general, the leaving group Y² in the compounds of formula (V) should be such that it provides sufficient reactivity with the amino group in the compounds of formula (VI). Compounds of formula (V) that contain such suitable leaving groups may be prepared, isolated and/or purified, and subsequently reacted with the compounds of formula (VI). Alternatively, compounds of formula (V) with suitable leaving groups may be prepared and further reacted without isolation or further purification with the compounds of formula (VI) to afford compounds of formula (I). Among suitable leaving groups in the compounds of formula (V) are halides, aromatic heterocycles, sulfonic acid esters, phosphoric acid esters, anhydrides, or groups derived from the reaction of compounds of formula (V) wherein Y² is hydroxy with reagents such as carbodiimides or carbodiimide species. Examples of suitable leaving groups include, but are not limited to, chloride, iodide, imidazole, —OC(O)alkyl, —OC(O)aryl, —OC(O)Oalkyl, —OC(O)Oaryl, —OS(O₂)alkyl, —OS(O₂)aryl, —OPO(Oaryl)₂, —OPO(Oalkyl)₂, and those derived from the reaction of the compounds of formula (V), wherein Y² is —OH, with carbodiimides.

Compounds of formula (V) where in Y² is a halogen can be prepared from compounds of formula (V) wherein Y² is hydroxy by reaction with a suitable agent. For example, the compounds of formula (V) wherein Y² is chloro may be prepared from compounds of formula (V) wherein Y² is hydroxy by reaction with agents such as thionyl chloride or oxalyl chloride. These reactions may be performed in the presence of a suitable base such as sodium carbonate, sodium bicarbonate, potassium carbonate, potassium bicarbonate, sodium hydroxide, potassium hydroxide, a tralkylamine, triethylamine for example, or a heteroaromatic base, pyridine for example. The resulting compounds may be isolated and then further reacted with the compounds of formula (VI) or they may be formed in situ and reacted with the compounds of formula (VI) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Compounds of formula (VI), 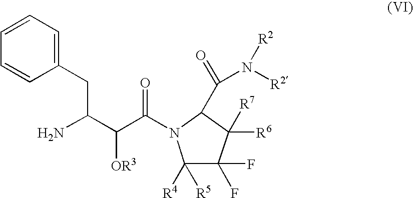
wherein R², R^{2′}, R³, R⁴, R⁵, R⁶, and R⁷ are as hereinbefore defined, may be prepared from reaction of compounds of formula (VII), 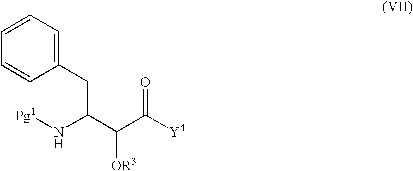
wherein Pg¹ is a suitable nitrogen protecting group, Y⁴ is hydroxy or a suitable leaving group, and R³ is as hereinbefore defined, with a compound of formula (III), wherein R², R^{2′}, R⁴, R⁵, R⁶, and R⁷ are as hereinbefore defined, or a salt or solvate thereof.

A suitable protecting group Pg¹ in the compounds of formula (VII) is one that is stable to subsequent reaction conditions in which the compounds of formula (VII) are allowed to react with the compounds of formula (III). Furthermore, such a protecting group should be chosen such that it can be removed after the compounds of formula (VII) have been allowed to react with the compounds of formula (III) to afford an intermediate compound that is subsequently deprotected to afford a compound of formula (VI). Suitable protecting groups include, but are not limited to, carbamates such as t-butyloxycarbonyl and benzyloxycarbonyl, imides such as phthaloyl, or suitable benzyl groups. Such protecting groups can be introduced into the compounds of formula (VII) and subsequently removed to provide compounds of formula (VI) according to methods known to those of ordinary skill in the art and as found in, for example, Greene, et al., Protective Groups in Organic Synthesis; John Wiley & Sons: New York, (1999).

In general, the leaving group Y⁴ in the compounds of formula (VII) should be such that it provides sufficient reactivity with the amino group in the compounds of formula (III). Compounds of formula (VII) that contain such suitable leaving groups may be prepared, isolated and/or purified, and subsequently reacted with the compounds of formula (III). Alternatively, compounds of formula (VII) with suitable leaving groups may be prepared and further reacted without isolation or further purification with the compounds of formula (III) to afford compounds of formula (VI). Among suitable leaving groups in the compounds of formula (VII) are halides, aromatic heterocycles, sulfonic acid esters, phosphoric acid esters, anhydrides, or groups derived from the reaction of compounds of formula (VII) wherein Y⁴ is hydroxy with reagents such as carbodiimides or carbodiimide species. Examples of suitable leaving groups include, but are not limited to, chloride, iodide, imidazole, —OC(O)alkyl, —OC(O)aryl, —OC(O)Oalkyl, —OC(O)Oaryl, —OS(O₂)alkyl, —OS(O₂)aryl, —OPO(Oaryl)₂, —OPO(Oalkyl)₂, and those derived from the reaction of the compounds of formula (VII), wherein Y⁴ is —OH, with carbodiimides.

Compounds of formula (VII) where in Y⁴ is a halogen can be prepared from compounds of formula (VII) wherein Y⁴ is hydroxy by reaction with a suitable agent. For example, the compounds of formula (VII) wherein Y⁴ is chloro may be prepared from compounds of formula (VII) wherein Y⁴ is hydroxy by reaction with agents such as thionyl chloride or oxalyl chloride. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (III) or they may be formed in situ and reacted with the compounds of formula (III) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Compounds of formula (VII) where in Y⁴ is an aromatic heterocycle can be prepared from compounds of formula (VII) wherein Y⁴ is hydroxy by reaction with a suitable agent such as carbonyl diimidazole. These compounds may be isolated and then further reacted with the compounds of formula (III) or they may be formed in situ and reacted with the compounds of formula (III) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the skill of one of ordinary skill in the art.

Compounds of formula (VII) wherein Y⁴ is —OC(O)alkyl or —OC(O)aryl may be prepared from compounds of formula (VII) wherein Y⁴ is hydroxy by reaction with suitable reagents such acyl halides, acyl imidazoles, or carboxylic acid under dehydrating conditions. Suitable reagents may include, but are not limited to, pivaloyl chloride, acetyl chloride, acetyl iodide formed in situ from acetyl chloride and sodium iodide, acetyl imidazole, or acetic acid under dehydrating conditions. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (III) or they may be formed in situ and reacted with the compounds of formula (III) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Compounds of formula (VII) wherein Y⁴ is —OC(O)Oalkyl, —OC(O)Oaryl can be prepared from compounds of formula (VII) wherein Y⁴ is hydroxy by reaction with a suitable agents such as chloroformates of the formula Cl—C(O)Oalkyl or Cl—C(O)Oaryl. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (III) or they may be formed in situ and reacted with the compounds of formula (III) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Compounds of formula (VII) wherein Y⁴ is —OS(O₂)alkyl or —OS(O₂)aryl can be prepared from compounds of formula (VII) wherein Y⁴ is hydroxy by reaction with a suitable agent such as an alkyl or aryl sulfonyl chloride. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (III) or they may be formed in situ and reacted with the compounds of formula (III) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Alternatively, compounds of formula (VI) may be prepared by reaction of compounds of formula (VII), wherein Y⁴ is —OH, with compounds of formula (III) under dehydrating conditions, followed by deprotection. These reactions may be performed using agents such as carbodiimides or carbodiimide derived species Such suitable agents include, but are not limited to, dicyclohexylcarbodiimide, diisopropylcarbodiimide, 1-[3-(dimethylamino)propyl]-3-ethylcarbodiimide hydrochloride (EDC), 2-chloro-4,6-dimethoxy-1,3,5-triazine (CDMT), cyanuric chloride, 4-(4,6-dimethoxy-1,3,5-triazin-2-yl)-4-methylmorpholinium chloride, 0-(7-azabenzotriazol-1-yl)-N,N,N′,N′-tetramethyluronium hexafluorophosphate (HATU), carbonyldiimidazole (CDI), benzotriazole-1-yl-oxy-tris-(dimethylamino)-phosphoniumhexafluorophosphate (BOP), 2-ethoxy-1-ethoxycarbonyl-1,2-dihydroquinoline (EEDQ), 2-(1H-benzotriazole-1-yl)-1,1,3,3-tetramethyluronium hexafluorophosphate (HBTU), 2-(1H-benzotriazole-1-yl)-1,1,3,3-tetramethyluronium tetrefluoroborate (TBTU), and 3-(diethoxyphosphoryloxy)-1,2,3-benzotriazin-4(3H)-one (DEPBT). These reactions may be performed in the presence of optional additives. Suitable additives include, but are not limited to, hydroxybenzotriazole (HOBt), hydroxyazabenzotriazole (HOAt), N-hydroxysuccinimide (HOSu), N-hydroxy-5-norbornene-endo-2,3-dicarboximide (HONB), and 4-dimethylaminopyridine (DMAP). Whether these additives are necessary depends on the identity of the reactants, the solvent, and the temperature. Such choices are within the knowledge of one of ordinary skill in the art.

Alternatively, the compounds of formula (I) may be prepared by reaction of a compound of formula (VIII), 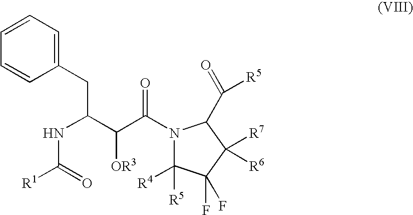
wherein Y⁵ is hydroxy or a suitable leaving group, and R¹, R³, R⁴, R⁵, R⁶, and R⁷ are as hereinbefore defined, with a compound of formula (IX), 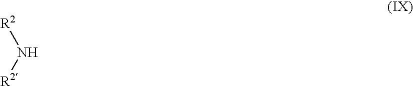
wherein R² and R^{2′} are hereinbefore defined, or a salt or solvate thereof.

In general, the leaving group Y⁵ in the compounds of formula (VIII) should be such that it provides sufficient reactivity with the amino group in the compounds of formula (IX). Compounds of formula (VIII) that contain such suitable leaving groups may be prepared, isolated and/or purified, and subsequently reacted with the compounds of formula (IX). Alternatively, compounds of formula (VIII) with suitable leaving groups may be prepared and further reacted without isolation or further purification with the compounds of formula (IX) to afford compounds of formula (I). Among suitable leaving groups in the compounds of formula (VIII) are halides, aromatic heterocycles, sulfonic acid esters, anhydrides, or groups derived from the reaction of compounds of formula (VIII) wherein Y⁵ is hydroxy with reagents such as carbodiimides or carbodiimide species. Examples of suitable leaving groups include, but are not limited to, chloride, iodide, imidazole, —OC(O)alkyl, —OC(O)aryl, —OC(O)Oalkyl, —OC(O)Oaryl, —OS(O₂)alkyl, —OS(O₂)aryl, —OPO(Oalkyl)₂, —OPO(Oaryl)₂, and those derived from the reaction of the compounds of formula (VIII), wherein Y⁵ is —OH, with carbodiimides.

Compounds of formula (VIII) where in Y⁵ is a halogen can be prepared from compounds of formula (VIII) wherein Y⁵ is hydroxy by reaction with a suitable agent. For example, the compounds of formula (VIII) wherein Y⁵ is chloro may be prepared from compounds of formula (VIII) wherein Y⁵ is hydroxy by reaction with agents such as thionyl chloride or oxalyl chloride. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (IX) or they may be formed in situ and reacted with the compounds of formula (IX) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Compounds of formula (VII) where in Y⁵ is an aromatic heterocycle can be prepared from compounds of formula (VII) wherein Y⁵ is hydroxy by reaction with a suitable agent such as carbonyl diimidazole. These compounds may be isolated and then further reacted with the compounds of formula (IX) or they may be formed in situ and reacted with the compounds of formula (IX) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Compounds of formula (VIII) wherein Y⁵ is —OC(O)alkyl or —OC(O)aryl may be prepared from compounds of formula (VIII) wherein Y⁵ is hydroxy by reaction with suitable reagents such acyl halides, acyl imidazoles, or carboxylic acid under dehydrating conditions. Suitable reagents may include, but are not limited to, pivaloyl chloride, acetyl chloride, acetyl iodide formed in situ from acetyl chloride and sodium iodide, acetyl imidazole, or acetic acid under dehydrating conditions. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (IX) or they may be formed in situ and reacted with the compounds of formula (IX) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Compounds of formula (VIII) wherein Y⁵ is —OC(O)Oalkyl, —OC(O)Oaryl can be prepared from compounds of formula (VIII) wherein Y⁵ is hydroxy by reaction with a suitable agents such as chloroformates of the formula Cl—C(O)Oalkyl or Cl—C(O)Oaryl. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (IX) or they may be formed in situ and reacted with the compounds of formula (IX) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Compounds of formula (VIII) wherein Y⁵ is —OS(O₂)alkyl or —OS(O₂)aryl can be prepared from compounds of formula (VIII) wherein Y⁵ is hydroxy by reaction with a suitable agent such as an alkyl or aryl sulfonyl chloride. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (IX) or they may be formed in situ and reacted with the compounds of formula (IX) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Alternatively, compounds of formula I may be prepared by reaction of compounds of formula (VIII), wherein Y⁵ is —OH, with compounds of formula (IX) under dehydrating conditions using agents such as carbodiimides or carbodiimide derived species. Such suitable agents include, but are not limited to, dicyclohexylcarbodiimide, diisopropylcarbodiimide, 1-[3-(dimethylamino)propyl]-3-ethylcarbodiimide hydrochloride (EDC), 2-chloro-4,6-dimethoxy-1,3,5-triazine (CDMT), cyanuric chloride, 4-(4,6-dimethoxy-1,3,5-triazin-2-yl)-4-methylmorpholinium chloride, O-(7-azabenzotriazol-1-yl)-N,N, N′,N′-tetramethyluronium hexafluorophosphate (HATU), carbonyldiimidazole (CDI), benzotriazole-1-yl-oxy-tris-(dimethylamino)-phosphoniumhexafluorophosphate (BOP), 2-ethoxy-1-ethoxycarbonyl-1,2-dihydroquinoline (EEDQ), 2-(1H-benzotriazole-1-yl)-1,1,3,3-tetramethyluronium hexafluorophosphate (HBTU), 2-(1H-benzotriazole-1-yl)-1,1,3,3-tetramethyluronium tetrefluoroborate (TBTU), and 3-(diethoxyphosphoryloxy)-1,2,3-benzotriazin-4(3H)-one (DEPBT). These reactions may be performed in the presence of optional additives. Suitable additives include, but are not limited to, hydroxybenzotriazole (HOBt), hydroxyazabenzotriazole (HOAt), N-hydroxysuccinimide (HOSu), N-hydroxy-5-norbornene-endo-2,3-dicarboximide (HONB), and 4-dimethylaminopyridine (DMAP). Whether these additives are necessary depends on the identity of the reactants, the solvent, and the temperature. Such choices are within the knowledge of one of ordinary skill in the art.

Compounds of formula (IX) are either commercially available or can be prepared by methods described herein or methods known to those of ordinary skill in the art.

Amorphous (2S)-4,4-difluoro-1-[(2S, 3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide can be prepared by working up the final deprotection reaction using standard conditions and removing the solvents under vacuum (as described in Example 4 which follows).

Crystalline (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide can be prepared by allowing amorphous (2S)-4,4-difluoro-1-[(2S,3S)-2-hydroxy-3-(3-hydroxy-2-methyl-benzoylamino)₄-phenyl-butyryl]-3,3-dimethyl-pyrrolidine-2-carboxylic acid (2,2,2-trifluoroethyl)-amide to stir in the presence of water (30 mg/mL), in the form of a slurry, at a temperature of from about 50° C. to about 75° C., preferably about 60° C., for a time period of between about 6 hours to about 48 hours, preferably about 16 hours. The resulting slurry can then be allowed to cool to room temperature and filtered to provide a solid. The solid may be further dried in a vacuum oven at a temperature between about 30° C. to about 60° C., preferably 40° C., for a time period of from about 2 hours to about 24 hours, preferably about 2 hours, and at an atmospheric pressure of about 30 psi.

Amorphous (2S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide can be prepared by working up the final deprotection reaction using standard conditions and removing the solvents under vacuum.

Crystalline (2S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide can be prepared by allowing amorphous (2S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide to stir in the presence of water (30 mg/mL), in the form of a slurry, at a temperature of from about 50° C. to about 75° C., preferably about 60° C., for a time period of between about 6 hours to about 48 hours, preferably about 16 hours. The resulting slurry can then be allowed to cool to room temperature and filtered to provide a solid. The solid may be further dried in a vacuum oven at a temperature between about 30° C. to about 60° C., preferably 40° C., for a time period of from about 2 hours to about 24 hours, preferably about 2 hours, and at an atmospheric pressure of about 30 psi.

Powder X-ray diffraction patterns may be obtained using a Bruker AXS D8 Discover diffractometer equipped with a Cu X-ray source operated at 40 kV and 50 mA at a mono cap of 0.5 mm. Samples (approximately 2 to 15 mg) are laid on a glass plate and slightly flattened with a spatula. The plate is put on the stage and a preset script is used to run the sample, the script instructs the system to perform an auto alignment for X, Y and Z stages. During analysis the sample is analyzed from angles of 4°-40° (2θ). The run time is selected at two times each 60 second and an oscillation value of 1. The stage oscillation will minimize crystal orientation effects.

Alternatively, powder X-ray diffraction patterns may be obtained using a Shimadzu XRD-6000 X-ray diffractometer equipped with a Cu X-ray source operated at 40 kV and 50 mA. Samples (approximately 10 to 30 mg) are laid on a Silicone plate to give no background signal. The sample is placed on the plate and then packed and smoothed with a glass slide on a sample holder. During analysis the samples are rotated at 60 rpm with a continuous scan mode and analyzed from angles of 4°-40° (2θ) at 5°/min with a 0.04° step. If limited material is available samples may be placed on a silicon plate (zero-background) and analyzed without rotation.

Alternatively, powder X-ray diffraction patterns may be obtained using a Bruker AXS D8 Advance diffractometer. Samples (approximately 100 mg) are packed in Lucite sample cups fitted with Si(511) plates as the bottom of the cup to give no background signal. Samples are spun in the (p plane at a rate of 30 rpm to minimize crystal orientation effects. The X-ray source (KCu_{α}, λ=1.54 Å) is operated at a voltage of 45 kV and a current of 40 mA. Data for each sample are collected over a period of 27 minutes in continuous detector scan mode at a scan speed of 1.8 seconds/step and a step size of 0.04°/step. Diffractograms are collected over the 2θ range of 4° to 30°.

Alternatively, powder X-ray diffraction patterns may be obtained using a Bruker AXS D8 Advance diffractometer X-ray equipped with a Cu X-ray source operated at 40 kV and 50 mA. During analysis the samples were rotated at 60 rpm and analyzed from angles of 4°-40° (θ-2θ). Samples (approximately 100 mg) were packed in Lucite sample cups fitted with Si (511) plates as the bottom of the cup to give no background signal. Samples were spun in the (p plane at a rate of 30 rpm to minimize crystal orientation effects. The x-ray source (KCu_{α}, λ=1.54 Å) was operated at a voltage of 45 kV and a current of 40 mA. Data for each sample were collected over a period of about 1 to 2 minutes in continuous detector scan mode at a scan speed of 1.8 seconds/step and a step size of 0.04°/step. Diffractograms were collected over the 20 range of 4° to 40°.

The examples and preparations provided below further illustrate and exemplify the methods of the present invention. It is to be understood that the scope of the present invention is not limited in any way by the scope of the following examples. In the following examples compounds with single or multiple stereoisomeric centers, unless otherwise noted, are at least 95% stereochemically pure.

### EXAMPLES

In the examples described below, unless otherwise indicated, all temperatures in the following description are in degrees Celsius (° C.) and all parts and percentages are by weight, unless indicated otherwise.

Various starting materials and other reagents were purchased from commercial suppliers, such as Aldrich Chemical Company or Lancaster Synthesis Ltd., and used without further purification, unless otherwise indicated.

The reactions set forth below were performed under a positive pressure of nitrogen, argon or with a drying tube, at ambient temperature (unless otherwise stated), in anhydrous solvents. Analytical thin-layer chromatography was performed on glass-backed silica gel 60° F. 254 plates (Analtech (0.25 mm)) and eluted with the appropriate solvent ratios (v/v). The reactions were assayed by high-pressure liquid chromotagraphy (HPLC) or thin-layer chromatography (TLC) and terminated as judged by the consumption of starting material. The TLC plates were visualized by UV, phosphomolybdic acid stain, or iodine stain.

¹H-NMR spectra were recorded on a Bruker instrument operating at 300 MHz and ¹³CNMR spectra were recorded at 75 MHz. NMR spectra are obtained as DMSO-d₆ or CDCl₃ solutions (reported in ppm), using chloroform as the reference standard (7.25 ppm and 77.00 ppm) or DMSO-d₆ (2.50 ppm and 39.52 ppm). Other NMR solvents were used as needed. When peak multiplicities are reported, the following abbreviations are used: s=singlet, d=doublet, t=triplet, m=multiplet, br=broadened, dd=doublet of doublets, dt=doublet of triplets. Coupling constants, when given, are reported in Hertz.

Infrared spectra were recorded on a Perkin-Elmer FT-IR Spectrometer as neat oils, as KBr pellets, or as CDCl₃ solutions, and when reported are in wave numbers (cm⁻¹). The mass spectra were obtained using LC/MS or APCI. All melting points are uncorrected.

All final products had greater than 95% purity (by HPLC at wavelengths of 220 nm and 254 nm).

In the following examples and preparations, “Et” means ethyl, “Ac” means acetyl, “Me” means methyl, “Ph” means phenyl, (PhO)₂POCI means chlorodiphenylphosphate, “HCl” means hydrochloric acid, “EtOAc” means ethyl acetate, “Na₂CO₃” means sodium carbonate, “NaOH” means sodium hydroxide, “NaCl” means sodium chloride, “NEt₃” means triethylamine, “THF” means tetrahydrofuran, “DIC” means diisopropylcarbodiimide, “HOBt” means hydroxy benzotriazole, “H₂O” means water, “NaHCO₃” means sodium hydrogen carbonate, “K₂CO₃” means potassium carbonate, “MeOH” means methanol, “i-PrOAc” means isopropyl acetate, “MgSO₄” means magnesium sulfate, “DMSO” means dimethylsulfoxide, “AcCl” means acetyl chloride, “CH₂Cl₂” means methylene chloride, “MTBE” means methyl t-butyl ether, “DMF” means dimethyl formamide, “SOCl₂” means thionyl chloride, “H₃PO₄” means phosphoric acid, “CH₃SO₃H” means methanesulfonic acid, “Ac₂O” means acetic anhydride, “CH₃CN” means acetonitrile, and “KOH” means potassium hydroxide.

### Example 1

### Preparation of (2S,3S)-343-acetoxy-2-methyl-benzoylamino)-2-hydroxy-4-phenyl-butyric Acid

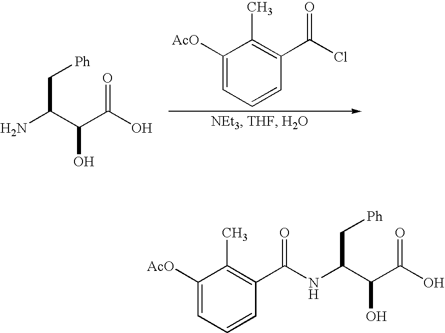

(2S,3S)-3-Amino-2-hydroxy-4-phenyl-butyric acid (which can be prepared according to the method of Pedrosa, et al., Tetrahedron Asymm. 2001, 12, 347; M. Shibasaki, et al., Tetrahedron Lett. 1994, 35, 6123; and Ikunaka, M., et al. Tetrahedron Asymm. 2002, 13, 1201; 185 g; 948 mmol) was added to a 5-L flask and was suspended in THF (695 mL). H₂O (695 mL) was poured in, followed by NEt₃ (277 mL; 1990 mmol). After stirring for 45 min, the solution was cooled to 6° C. A solution of acetic acid 3-chlorocarbonyl-2-methyl-phenyl ester (201 g; 948 mmol) in THF (350 mL) was then added dropwise. One-half hour later, the pH was adjusted from 8.7 to 2.5 with 6 N HCl (170 mL). Solid NaCl (46 g) was added, the ice bath was then removed and the mixture was stirred vigorously while warming to room temperature. The mixture was transferred to 4-L separatory funnel, using 1:1 THF/H₂O (50 mL) for the transfer, and the lower aqueous phase was then removed. The organic fraction was transferred to a 5-L distillation flask, and was then diluted with fresh THF (2.5 L). The solution was azeotropically dried and concentrated to a volume of 1.3 L by distillation of THF at one atmosphere. To complete the azeotropic drying, fresh THF (2.0 L) was added and the solution was concentrated to 1.85 L by distillation at one atmosphere and was then held at 55° C. n-Heptane (230 mL) was added dropwise via addition funnel and the solution was then immediately seeded. After crystallization had initiated, additional n-heptane (95 mL) was added dropwise. The resulting crystal slurry was stirred vigorously for 7 min. Additional n-heptane (1.52 L) was then added as a slow stream. The crystal slurry was then allowed to cool to room temperature slowly and stir overnight. The suspension was vacuum-filtered and the filter cake was then washed with 1:1 THF/n-heptane (700 mL). After drying in a vacuum oven at 45-50° C., 324 g (92%) of (2S,3S)-3-(3-acetoxy-2-methyl-benzoylamino)-2-hydroxy-4-phenyl-butyric acid was obtained as a crystalline solid contaminated with ˜7 mol % Et₃N.HCl: mp=189-191° C., ¹H NMR (300 MHz, DMSO-d₆) δ 12.65 (br s, 1H), 3.80 (d, J=9.7 Hz, 1H), 7.16-7.30 (m, 6H), 7.07 (dd, J=1.1, 8.0 Hz, 1H), 7.00 (dd, J=1.1, 7.5 Hz), 4.40-4.52 (m, 1H), 4.09 (d, J=6.0 Hz, 1H), 2.92 (app dd, J=2.9, 13.9 Hz, 1H), 2.76 (app dd, J=11.4, 13.9 Hz, 1H), 2.29 (s, 3H), 1.80 (s, 3H); ¹³C NMR (75 MHz, DMSO-d₆) δ 174.4, 169.3, 168.1, 149.5, 139.7, 139.4, 129.5, 128.3, 127.9, 126.5, 126.3, 124.8, 123.3, 73.2, 53.5, 35:4, 20.8, 12.6; MS (CI) m/z 372.1464 (372.1447 calcd for C₂₀H₂₂NO₆, M+H⁺); elemental analysis calcd for C₂₀H₂₁NO₆.0.07 Et₃N.HCl: C, 64.34; H, 5.86; N, 3.95; Cl, 0.70; found: C, 64.27; H, 5.79; N, 3.96; Cl; 0.86.

### Example 2

### Preparation of (2S,3S)-2-acetoxy-3-(3-acetoxy-2-methyl-benzoylamino)-4-phenyl-butyric Acid

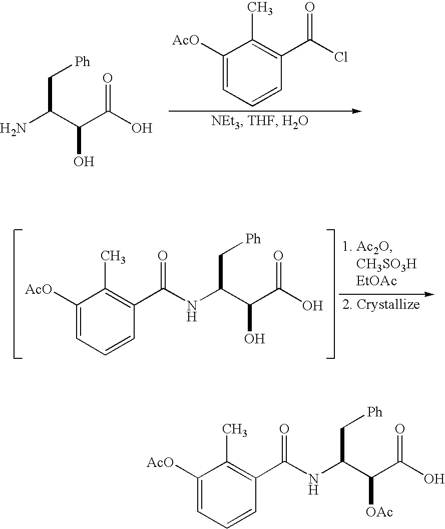

A mixture of (2S,3S)-3-Amino-2-hydroxy-4-phenyl-butyric acid (110 kg, 563 mol), NaCl (195 kg), and THF (413 L) was charged with NEt₃ (120 kg, 1183 mol) and H₂O (414 L) at ambient temperature. The resulting mixture was cooled to 0° C. Acetic acid 3-chlorocarbonyl-2-methyl-phenyl ester (120 kg, 563 mol) was added to a separate reactor and was then dissolved in THF (185 L). The resulting solution of acetic acid 3-chlorocarbonyl-2-methyl-phenyl ester was cooled to 10° C., and was then added to the (2S,3S)-3-amino-2-hydroxy-4-phenyl-butyric acid mixture while maintaining the temperature <10° C. during addition. The resulting biphasic mixture was agitated at 5° C. for 1 h, and was then adjusted to pH 2.5-3.0 with concentrated HCl (62 kg). The mixture was then warmed to 25° C., and the layers were separated. The resulting THF fraction, containing (2S,3S)-3-(3-acetoxy-2-methyl-benzoylamino)-2-hydroxy-4-phenyl-butyric acid, was partially concentrated by distillation at one atmosphere. THF was then replaced with ethyl acetate by distillation at one atmosphere, while maintaining a minimum pot volume of 1500 L. The resulting solution was cooled to 25° C., and was then charged with acetic anhydride (74.8 kg, 733 mol) and methanesulfonic acid (10.8 kg, 112 mol). The mixture was heated at 70° C. for approximately 3 h. The mixture was cooled to 25° C., and was then quenched with H₂O (1320 L) while maintaining the temperature at 20° C. After removal of the aqueous layer, the organic fraction was charged with ethyl acetate (658 L) and H₂O (563 L). After agitation, the aqueous phase was removed. The organic fraction was washed twice with 13 wt. % aqueous NaCl (2×650 L). The organic fraction was partially concentrated and dried by vacuum distillation (70-140 mm Hg) to a volume of approximately 1500 L. The resulting solution was heated to 40° C., and was then charged with n-heptane (1042 L) while maintaining the temperature at 40° C. The solution was seeded with (2S,3S)-2-acetoxy-3-(3-acetoxy-2-methyl-benzoylamino)₄-phenyl-butyric acid (0.1 kg), and additional n-heptane (437 L) was then added slowly. The crystallizing mixture was maintained at 40° C. for 1 h. Additional n-heptane (175 L) was added while maintaining the temperature at 40° C. The crystalline suspension was cooled and held at 25° C. for 1 h, then at 0° C. for 2 h. The suspension was filtered, using n-heptane for rinsing. The wet cake was dried under vacuum at 55° C. to give 174 kg (74.5%) of (2S,3S)-2-acetoxy-3-(3-acetoxy-2-methyl-benzoylamino)-4-phenyl-butyric acid as a white solid: m.p.=152-154° C.; ¹H NMR (300 MHz, CDCl₃) δ 7.21-7.35 (m, 5H), 7.13 (app t, J=7.9 Hz, 1H), 7.01 (app d, J=8.1 Hz, 1H), 6.94 (app d, J=7.2 Hz, 1H), 5.99 (d, J=9.0 Hz, 1H), 5.33 (d, J=4.1 Hz, 1H), 4.96-5.07 (m, 1H), 3.07 (dd, J=5.5, 14.6 Hz, 1H), 2.90 (dd, J=10.0, 14.5 Hz, 1H), 2.30 (s, 3H), 2.18 (s, 3H), 1.96 (s, 3H); ¹³C NMR (125 MHz, CDCl₃) δ 170.4, 170.2, 169.6, 169.5, 149.5, 137.81, 136.5, 129.2, 128.6, 128.4, 127.0, 126.6, 124.5, 123.7, 73.1, 50.9, 35.9, 20.6, 20.5, 12.4; elemental analysis calcd for C₂₂H₂₃NO₇: C, 63.92; H, 5.61; N, 3.39; found: C, 64.22; H, 5.68; N, 3.33; MS (CI) m/z 414.1572 (414.1553 calcd for C₂₂H₂₄NO₇, M+H⁺).

### Example 3

### Preparation of (2S)-4,4-difluoro-3,3-dimethyl-pyrrolidine-2-carboxylic acid (2,2,2-trifluoro-ethyl)-amide; hydrochloride

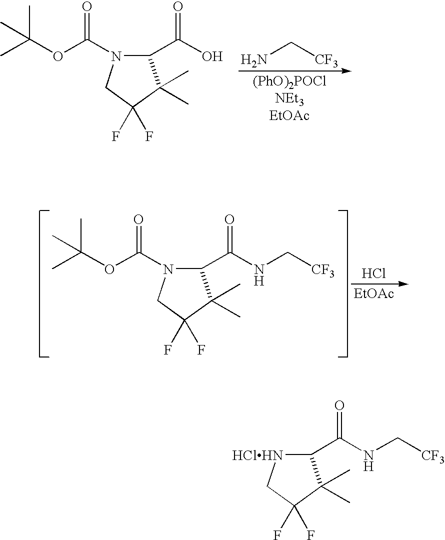

NEt₃ (75.2 g, 743 mmol) was slowly added to a 10° C. solution of (2S)-4,4-difluoro-3,3-dimethyl-pyrrolidine-1,2-dicarboxylic acid 1-tert-butyl ester (98.3 g, 352 mmol), chlorodiphenylphosphate (101 g, 376 mmol), and ethyl acetate (1.0 L). The mixture was warmed to ambient temperature for 45 min., and was then cooled to 10° C. 2,2,2-Trifluoroethylamine (39.5 g, 399 mmol) was slowly added and the resultant mixture was stirred at ambient temperature for 2.75 h. 20% Aqueous citric acid (1.0 L) was added and the resulting layers were separated. The aqueous fraction was extracted with ethyl acetate (2×300 mL). The combined organic fractions were washed with saturated aqueous NaHCO₃ (2×500 mL), and then with saturated aqueous NaCl (300 mL). The resulting organic fraction was concentrated to a weight of 900 g using a rotary evaporator. A 3 N HCl/ethyl acetate solution (500 mL) was added to the concentrate, and the mixture was stirred at ambient temperature for 24 h. The resulting solid was filtered, washed with ethyl acetate (100 mL), and was then dried in a vacuum oven at 55° C. to provide 98.0 g (93.9%) of (2S)-4,4-difluoro-3,3-dimethyl-pyrrolidine-2-carboxylic acid (2,2,2-trifluoro-ethyl)-amide; hydrochloride as a white solid: ¹H NMR (300 MHz, DMSO-d₆) δ 10.46 (br s, 2H), 9.50 (t, J=6.2 Hz, 1H), 4.17-4.33 (m, 2H), 3.68-4.02 (m, 3H), 1.23 (app d, J=2.1 Hz, 3H), 0.97 (app d, J=2.0 Hz, 3H); ¹³C NMR (75 MHz, DMSO-d₆) δ 165.6, 127.9 (dd, J_{CF}=250.2, 257.2 Hz), 125.6 (q, J_{CF}=279.0 Hz), 64.8, 48.2 (t, J_{CF}=33.4 Hz), 45.7 (t, J_{CF}=21.2 Hz), 18.2 (d, J_{CF}=7.5 Hz), 17.2 (app dd, J_{CF}=2.3, 5.8 Hz); MS (CI) m/z 261.1015 (261.1026 calcd for C₉H₁₄N₂OF₅, M-HCl+H⁺); elemental analysis calcd for C₉H₁₄N₂OClF₅: C, 36.44; H, 4.76; N, 9.44; Cl, 11.95; F, 32.02; found: C, 36.45; H, 4.86; N, 9.43; Cl, 12.06; F, 32.15.

### Example 4

### Preparation of (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoro-ethyl)-amide

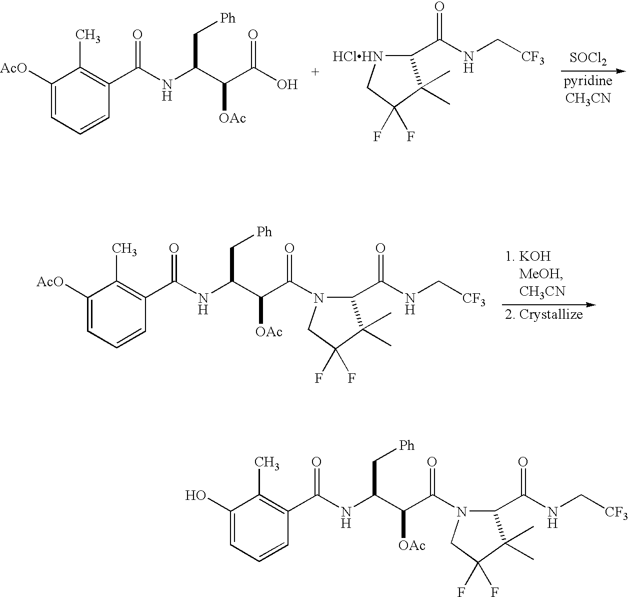

Pyridine (149 g, 1.89 mol) was added to a solution of (2S,3S)-2-acetoxy-3-(3-acetoxy-2-methyl-benzoylamino)-4-phenyl-butyric acid (193 g, 468 mmol) and acetonitrile (1.6 L) at ambient temperature, and the mixture was then cooled to 10° C. A solution of SOCl₂ (62.3 g, 523 mmol) and acetonitrile (50 mL) was added over 15 min., and cooling was then discontinued. 15 minutes later, additional SOCl₂ (0.80 g, 6.7 mmol) was added. After stirring at ambient temperature for 25 min., the mixture was cooled to 10° C. (2S)-4,4-Difluoro-3,3-dimethyl-pyrrolidine-2-carboxylic acid (2,2,2-trifluoro-ethyl)-amide; hydrochloride (139 g, 468 mmol) was added in portions over 15 min. The mixture was warmed to ambient temperature for 1 h, and was then cooled to 10° C. A 5° C. solution of KOH (85% assay; 186 g, 2.82 mol) and methanol (1.1 L) was then added over 10 min, followed by addition of K₂CO₃ (51.8 g, 375 mmol). The mixture was warmed to ambient temperature for 1 h, and was then concentrated to a weight of 1.5 kg using a rotary evaporator. The resulting mixture was partitioned between 0.5 N HCl (1.6 L) and ethyl acetate (1.4 L), and the layers were separated. The organic fraction was sequentially washed with saturated aqueous NaHCO₃ (1.4 L), 0.5 N HCl (1.6 L), and then H₂O (1.4 L). The organic fraction was concentrated to a wet solid using a rotary evaporator, and was then further dried in a vacuum oven at 50° C. for 24 h. The resulting solid was dissolved in absolute ethanol (800 mL), and was then concentrated on a rotary evaporator. The resulting solid was once again dissolved in ethanol (600 mL), then concentrated on a rotary evaporator, and then dried in a vacuum oven at 50° C. for 24 h. The solid was dissolved in ethanol and 0.11 N HCl (620 mL) was then slowly added. H₂O (950 mL) was slowly added and the resulting suspension of crystals was stirred overnight. The solid was filtered, washed with ethanol/H₂O (1:3, 200 mL), and dried in a vacuum oven at 55° C. to provide 259 g (96.9%) of (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoro-ethyl)-amide as a white crystalline solid: ¹H NMR (300 MHz, DMSO-d₆) displayed a 20:1 mixture of rotamers. Major rotamer resonances δ 9.34 (s, 1H), 8.66 (app t, J=6.3 Hz, 1H), 8.13 (d, J=8.3 Hz, 1H), 7.15-7.35 (m, 5H), 6.96 (app t, J=7.7 Hz, 1H), 6.79 (d, J=7.3 Hz, 1H), 6.55 (d, J=6.7 Hz, 1H), 5.56 (d, J=6.4 Hz, 1H), 4.264.54 (m, 5H), 3.814.07 (m, 2H), 2.86-2.90 (m, 1H), 2.71 (app dd, J=10.5, 13.6 Hz, 1H), 1.82 (s, 3H), 1.22 (s, 3H), 1.04 (s, 3H) [characteristic minor rotamer resonances δ 8.62 (5, J=6.5 Hz), 5.35 (d, J=7.6 Hz), 1.86 (s)]; ¹³C NMR (75 MHz, DMSO-d₆) displayed a 20:1 mixture of rotamers. Major rotamer resonances δ 171.5, 169.6, 168.6, 155.7, 139.6, 139.4, 129.8, 128.2, 127.9 (dd, J_{CF}=251.7, 253.5 Hz), 126.2, 126.0, 125.0 (q, J_{CF}=279.2 Hz), 121.8, 117.9, 115.6, 73.2, 68.3, 53.0, 51.4 (t, J_{CF}=32.6 Hz), 43.8 (t, J_{CF}=20.8 Hz), 34.5, 22.4 (d, J_{CF}=4.1 Hz), 16.9 (d, J_{CF}=7.3 Hz), 12.5 [characteristic minor rotamer resonances δ 171.7, 139.1, 129.5, 68.7, 47.0 (t), 16.5 (d)]; MS (CI) m/z 572.2189 (527.2184 calcd for C₂₇H₃₁N₃O₅F₅, M+H⁺); elemental analysis calcd for C₂₇H₃₀N₃O₅F₅: C, 56.74; H, 5.29; N, 7.35; F, 16.62; found: C, 56.50; H, 5.50; N, 7.15; F, 16.36.

### Example 6

### Preparation of Crystalline (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic Acid (2,2,2-trifluoro-ethyl)-amide

Amorphous (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoro-ethyl)-amide was allowed to stir with water (30 mg of compound per mL of water), in the form of a slurry, at a temperature of from about 50° C. to about 75° C., for about 6 hours to about 48 hours. The slurry was then cooled to room temperature and filtered. The remaining solid was dried in a vacuum oven between about 30° C. to about 60° C. for about 2 hours to about 24 hours under an atmospheric pressure of about 30 psi.

### Example 7

### X-ray Diffraction Pattern for Crystalline (2S)-4,4-difluoro-1-[(2S,3S)-2-hydroxy-3-(3-hydroxy-2-methyl-benzoylamino)-4-phenyl-butyrylo-3,3-dimethyl-pyrrolidine-2-carboxylic Acid (2,2,2-trifluoroethyl)-amide

Powder X-ray diffraction pattern for (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoro-ethyl)-amide were collected using a Bruker AXS D8 Advance diffractometer X-ray equipped with a Cu X-ray source operated at 40 kV and 50 mA. During analysis the samples were rotated at 60 rpm and analyzed from angles of 40-400 (0-20). Samples (approximately 10 mg) were packed in Lucite sample cups fitted with Si (511) plates as the bottom of the cup to give no background signal. Samples were spun in the (p plane at a rate of 30 rpm to minimize crystal orientation effects. The x-ray source (KCu_{α}, λ=1.54 Å) was operated at a voltage of 45 kV and a current of 40 mA. Data for each sample were collected over a period of about 1 to 2 minutes in continuous detector scan mode at a scan speed of 1.8 seconds/step and a step size of 0.04°/step. Diffractograms were collected over the 20 range of 4° to 40°. The results are summarized in table 1.

TABLE 1

| | Intensity |
|---|---|
| Angle 2-theta | (% of highest)* |
| 6.9 | 7.2 |
| 7.0 | 7.2 |
| 7.2 | 7.5 |
| 7.5 | 9.6 |
| 7.6 | 9.4 |
| 7.8 | 15.1 |
| 8.0 | 43.8 |
| 8.7 | 100.0 |
| 9.3 | 8.5 |
| 9.5 | 8.0 |
| 9.8 | 8.9 |
| 9.9 | 8.53 |
| 10.0 | 9.2 |
| 10.3 | 14.2 |
| 10.6 | 9.9 |
| 11.2 | 23.8 |
| 11.7 | 43.6 |
| 12.0 | 10.0 |
| 12.1 | 9.2 |
| 12.2 | 8.7 |
| 12.3 | 9.2 |
| 12.5 | 8.7 |
| 12.7 | 8.7 |
| 12.7 | 8.5 |
| 12.9 | 9.2 |
| 12.9 | 9.4 |
| 13.2 | 9.3 |
| 13.2 | 9.7 |
| 13.3 | 10.4 |
| 13.5 | 11.1 |
| 13.6 | 10.9 |
| 13.6 | 10.5 |
| 13.7 | 10.2 |
| 14.0 | 10.2 |
| 14.2 | 20.5 |
| 14.7 | 31.9 |
| 15.2 | 21.1 |
| 15.7 | 32.3 |
| 16.2 | 40.4 |
| 16.7 | 35.1 |
| 17.1 | 24.7 |
| 17.1 | 24.2 |
| 17.6 | 24.5 |
| 18.0 | 29.7 |
| 18.9 | 30.2 |
| 19.2 | 28.3 |
| 20.4 | 51.8 |
| 21.2 | 25.7 |
| 21.5 | 21.8 |
| 21.8 | 22.1 |
| 21.9 | 22.0 |
| 23.7 | 16.8 |
| 24.1 | 16.1 |
| 24.7 | 31.7 |
| 25.7 | 15.3 |
| 26.2 | 19.2 |
| 27.1 | 28.0 |
| 28.0 | 20.6 |
| 28.8 | 16.8 |
| 30.5 | 17.4 |
| 32.7 | 13.4 |
| 32.8 | 13.7 |
| 33.6 | 13.0 |
| 33.7 | 14.3 |
| 33.8 | 15.6 |
| 33.9 | 16.2 |
| 35.6 | 10.0 |
| 36.5 | 10.9 |

*The peak intensity may change depending on the crystalline size and habit

### Example 8

### Preparation of 3-acetoxy-2,5-dimethyl-benzoic Acid

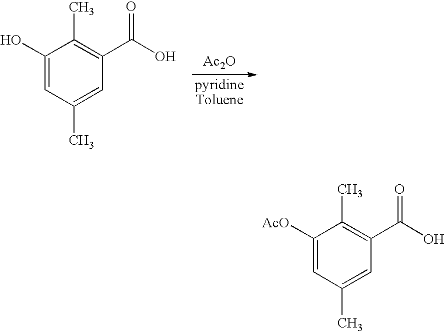

Pyridine (34.0 mL, 419 mmol) and acetic anhydride (150 mL, 1.59 mol) were sequentially added to a suspension of 3-hydroxy-2,5-dimethyl-benzoic acid (211 g, 1.27 mol) in toluene (1.05 L). The mixture was heated at 50° C. under argon for 6 h. Heating was discontinued and, while the mixture was still warm, n-heptane (2.10 L) was added. The mixture was allowed to cool and stir at ambient temperature overnight. The suspension was filtered, using n-heptane for rinsing, and the solid was dried in a vacuum oven at 50° C. to give 212 g (80.1%) of 3-acetoxy-2,5-dimethyl-benzoic acid as a pale yellow solid: m.p.=153-154° C.; ¹H NMR (300 MHz, CDCl₃) δ 11.5 (br s, 1H), 7.80 (s, 1H), 7.10 (s, 1H), 2.44 (s, 3H), 2.41 (s, 3H), 2.39 (s, 3H); ¹³C NMR (75 MHz, DMSO-d₆) δ 169.3, 168.8, 149.9, 136.3, 132.9, 128.4, 128.0, 126.3, 20.8, 20.5, 13.1; MS (CI) m/z 209.0822 (209.0814 calcd for C₁₁H₁₃O₄, M+H⁺); elemental analysis calcd for C₁₁H₁₂O₄: C, 63.45; H, 5.81; found: C, 63.54; H, 5.88.

### Example 9

### Preparation of Acetic Acid 3-chlorocarbonyl-2,5-dimethyl-phenyl Ester

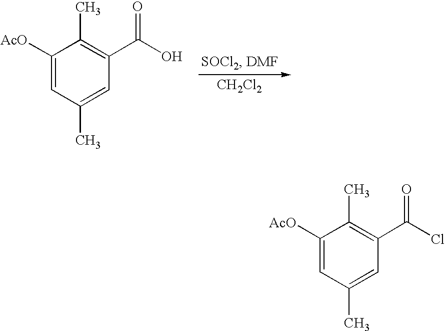

SOCl₂ (80.0 mL, 1.09 mol) was added to a suspension of 3-acetoxy-2,5-dimethyl-benzoic acid (206 g, 990 mmol), DMF (4.0 mL), and CH₂Cl₂ (1.03 L). The resulting mixture was stirred at ambient temperature for 1.5 h. n-Heptane (1.03 L) was added, followed by the slow addition of saturated aqueous NaHCO₃ (2.06 L), and the layers were then separated. The organic fraction was washed with saturated aqueous NaCl (1.00 L), dried over MgSO₄, filtered, and concentrated with a rotary evaporator to give 193 g (86.2%) of acetic acid 3-chlorocarbonyl-2,5-dimethyl-phenyl ester as a pale yellow solid: m.p.=52-54° C.; ¹H NMR (300 MHz, CDCl₃) δ 7.92 (s, 1H), 7.15 (s, 1H), 2.44 (s, 3H), 2.38 (s, 3H), 2.35 (s, 3H); ¹³C NMR (75 MHz, CDCl₃) δ 169.4, 167.7, 150.1, 137.3, 134.7, 132.0, 130.2, 129.1, 21.2, 21.1, 13.7; elemental analysis calcd for C₁₁H₁₁O₃Cl: C, 58.29; H, 4.89; found: C, 58.64; H, 4.89.

### Example 10

### Preparation of (2S,3S)-3-(3-Acetoxy-2,5-dimethyl-benzoylamino)-2-hydroxy-4-phenyl-butyric Acid

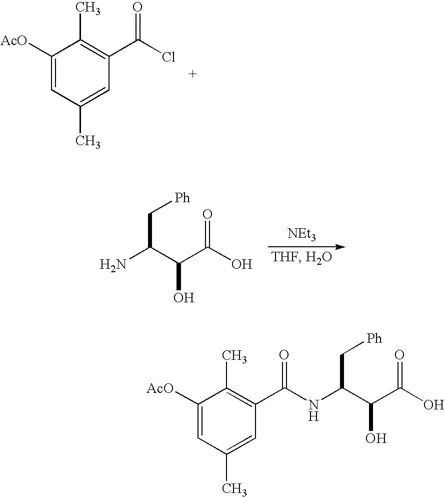

NEt₃ (265 mL, 1.88 mol) was added to a suspension of (2S,3S)-3-amino-2-hydroxy-4-phenyl-butyric acid (175 g, 896 mmol), tetrahydrofuran (875 mL), and H₂O (875 mL) at ambient temperature. The resulting solution was cooled to 0° C. A solution of acetic acid 3-chlorocarbonyl-2,5-dimethyl-phenyl ester (193 g, 854 mmol) and tetrahydrofuran (430 mL) was slowly added. One hour later, H₂O (225 mL) was added, followed by the slow addition of 3 N HCl (390 mL). The resulting mixture was allowed to slowly warm to ambient temperature with stirring overnight. The solid was filtered, using H₂O (430 mL) for rinsing. After drying in a vacuum oven at 50° C., 301 g (91.5%) of (2S,3S)-3-(3-acetoxy-2,5-dimethyl-benzoylamino)-2-hydroxy-4-phenyl-butyric acid was obtained as a white solid that was contaminated with 8 mol % Et₃N.HCl: m.p.=220-224° C.; ¹H NMR (300 MHz, DMSO-d₆) δ 12.65 (br s, 1H), 8.23 (d, J=9.0 Hz, 1H), 7.15-7.30 (m, 5H), 6.89 (s, 1H), 6.79 (s, 1H), 5.63 (br s, 1H), 4.39-4.50 (m, 1H), 4.07 (d, J=5.9 Hz, 1H), 2.91 (app dd, J=3.0, 14.0 Hz, 1H), 2.74 (app dd, J=11.1, 14.1 Hz, 1H), 2.27 (s, 3H), 1.24 (s, 3H), 1.72 (s, 3H) [characteristic resonances of Et₃N.HCl: δ 3.09 (q, J=7.3 Hz), 1.18 (t, J=7.3 Hz)]; ¹³C NMR (75 MHz, DMSO-d₆) δ 174.4, 169.2, 168.2, 149.4, 139.4, 135.9, 129.5, 128.3, 126.3, 125.6, 124.7, 123.5, 73.2, 53.5, 35.4, 20.8, 20.6, 12.2 [characteristic resonances of Et₃N.HCl: δ 45.9, 8.8]; MS (CI) m/z 386.1600 (386.1604 calcd for C₂₁H₂₄NO₆, M+H⁺); elemental analysis calcd for C₂₁H₂₃NO₆.0.08 Et₃N.HCl: C, 65.08; H, 6.17; N, 3.82; found: C, 64.88; H, 6.10; N, 3.68.

### Example 11

### Preparation of (2S,3S)-2-Acetoxy-3-(3-acetoxy-2,5-dimethyl-benzoylamino)-4-phenyl-butyric Acid

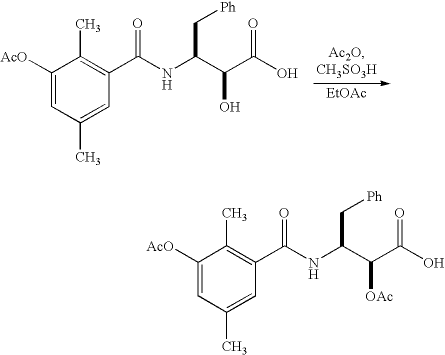

Methanesulfonic acid (16.5 mL, 253 mmol) and acetic anhydride (91.0 mL, 960 mmol) were sequentially added to a suspension of (2S,3S)-3-(3-acetoxy-2,5-dimethyl-benzoylamino)-2-hydroxy-4-phenyl-butyric acid (296 g, 768 mmol) in ethyl acetate (3.00 L) at ambient temperature. The mixture was heated at 75° C. for 2 h, and the resulting solution was then cooled to ambient temperature. The solution was sequentially washed with H₂O (2.0 L), half-saturated aqueous NaCl (2.0 L), and then with saturated aqueous NaCl (1.0 L). The resulting organic fraction was concentrated to approximately half volume by distillation at one atmosphere. Heating was discontinued and the solution was allowed to cool to ambient temperature to give a suspension. n-Heptane (3.0 L) was added and the suspension stirred at ambient temperature overnight. The solid was filtered, using 1:2 ethyl acetate/n-heptane (1.5 L) for rinsing. After drying in a vacuum oven at 50° C., 316 g (96.3%) of (2S,3S)-2-acetoxy-3-(3-acetoxy-2,5-dimethyl-benzoylamino)-4-phenyl-butyric acid was obtained as a white solid: m.p.=185-186° C.; ¹H NMR (300 MHz, DMSO-d₆) δ 13.3 (s, 1H), 8.49 (d, J=8.8 Hz, 1H), 7.19-7.34 (m, 5H), 6.91 (s, 1H), 6.71 (s, 1H), 5.11 (d, J=5.0 Hz, 1H), 4.61-4.72 (m, 1H), 2.79-2.90 (m, 2H), 2.27 (s, 3H), 2.24 (s, 3H), 2.14 (s, 3H), 1.73 (s, 3H); ¹³C NMR (75 MHz, DMSO-d₆) δ 170.3, 169.7, 169.2, 168.5, 149.4, 139.1, 138.5, 136.1, 129.4, 128.5, 126.6, 125.4, 124.7, 123.8, 73.9, 51.1, 35.2, 20.9, 20.8, 20.6, 12.1; MS (CI) m/z 428.1713 (428.1709 calcd for C₂₃H₂₆NO₇, M+H⁺); elemental analysis calcd for C₂₃H₂₅NO₇: C, 64.63; H, 5.90; N, 3.28; found: C, 64.79; H, 5.96; N, 3.15.

### Example 12

### Preparation of (2S)-4,4-Difluoro-3,3-dimethyl-pyrrolidine-2-carboxylic Acid Ethylamide; Hydrochloride

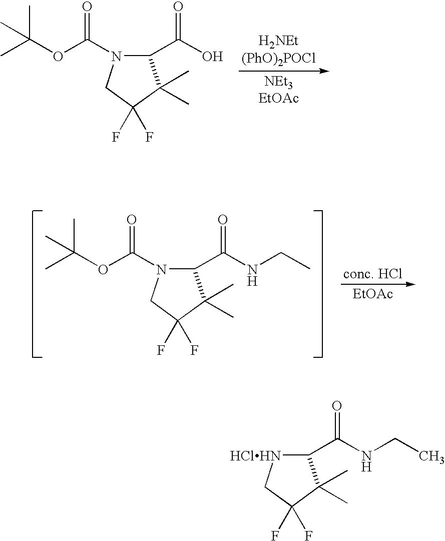

Chlorodiphenylphosphate (38.4 mL, 185 mmol) was added to a solution of (2S)-4,4-difluoro-3,3-dimethyl-pyrrolidine-1,2-dicarboxylic acid 1-tert-butyl ester (48.8 g, 175 mmol) in ethyl acetate (490 mL) at ambient temperature. The solution was cooled to 0° C., and NEt₃ (51.0 mL, 367 mmol) was added dropwise. Cooling was discontinued and the resulting suspension was allowed to warm to ambient temperature and stir for 1 h. The suspension was cooled to 0° C., and H₂NEt (96.0 mL of a 2.0 M solution in tetrahydrofuran, 192 mmol) was slowly added. The resulting mixture was allowed to warm to ambient temperature and stir for 2 h. 20% Aqueous citric acid (490 mL) was added and the layers were then separated. The aqueous fraction was extracted with ethyl acetate (125 mL). The combined organic fractions were washed with saturated aqueous NaHCO₃ (490 mL), and the layers were then separated. The aqueous fraction was extracted with ethyl acetate (125 mL). The combined organic fractions were washed with saturated aqueous NaCl (250 mL), dried over MgSO₄, and then concentrated to a volume of 500 mL using a rotary evaporator. Concentrated HCl (61.0 mL, 734 mmol) was added, and the solution was stirred at ambient temperature overnight. The resulting suspension was dried azeotropically with ethyl acetate (3×250 mL) by distillation at one atmosphere. The resulting suspension was cooled to ambient temperature, and was then filtered, using ethyl acetate (100 mL) for rinsing. After drying under vacuum at ambient temperature, 37.4 g (88.2%) of (2S)-4,4-difluoro-3,3-dimethyl-pyrrolidine-2-carboxylic acid ethylamide; hydrochloride was obtained as a white solid: m.p.=238-2390C (decomp.); ¹H NMR (300 MHz, DMSO-d₆) δ 10.3 (br s, 2H), 8.70 (t, J=5.3 Hz, 1H), 4.08 (s, 1H), 3.71-3.80 (m, 2H), 3.08-3.34 (m, 2H), 1.21 (app d, J=2.2 Hz, 3H), 1.08 (t, J=7.2 Hz, 3H), 0.97 (app d, J=2.1 Hz, 3H); ¹³C NMR (75 MHz, DMSO-d₆) δ 163.8, 128.1 (dd, JCF=248.6, 255.5 Hz), 64.8, 48.1 (t, J_{CF}=33.7 Hz), 45.5 (t, J_{CF}=20.8 Hz), 34.3, 18.3 (d, J_{CF}=7.4 Hz), 17.4 (app dd, J_{CF}=2.1, 5.4 Hz), 14.8; MS (CI) m/z 207.1317 (207.1309 calcd for C₉H₁₇N₂OF₂, M-HCl+H⁺); elemental analysis calcd for C₉H₁₇ClF₂N₂O: C, 44.54; H, 7.06; N, 11.54; F, 15.66; found: C, 44.40; H, 7.06; N, 11.65; F, 15.61.

### Example 13

### Preparation of Acetic Acid 3-{(1S,2S)-2-acetoxy-1-benzyl-3-[(2S)-2-ethylcarbamoyl-4,4-difluoro-3,3-dimethyl-pyrrolidin-1-yl]-3-oxo-propylcarbamoyl}-2,5-dimethyl-phenyl Ester

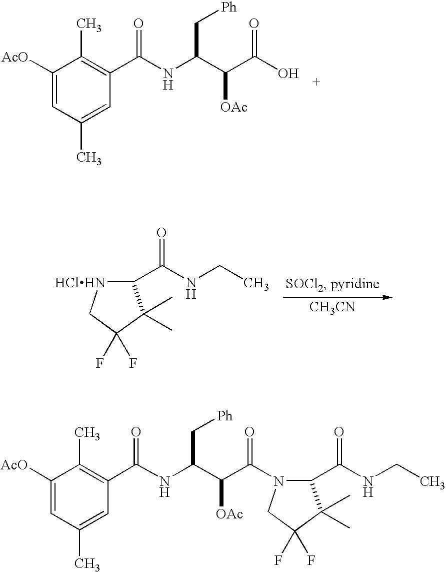

SOCl₂ (1.90 mL, 25.8 mmol) was added dropwise to a 0° C. solution of (2S,3S)-2-acetoxy-3-(3-acetoxy-2,5-dimethyl-benzoylamino)-4-phenyl-butyric acid (10.0 g, 23.5 mmol), pyridine (7.60 mL, 93.9 mmol), and CH₃CN (90.0 mL). The resulting solution was allowed to warm to ambient temperature for 1 h, then was cooled to 0° C. (2S)-4,4-Difluoro-3,3-dimethyl-pyrrolidine-2-carboxylic acid ethylamide; hydrochloride (5.71 g, 23.5 mmol) was added in one portion. The resulting solution was allowed to warm to ambient temperature and stir for 2.5 h. Saturated aqueous NaHCO₃ (110 mL) and methyl t-butyl ether (110 mL) were added, and the resulting layers were separated. The resulting organic fraction was sequentially washed with 20% aqueous citric acid (90 mL), saturated aqueous NaHCO₃ (70 mL), and saturated aqueous NaCl (70 mL). Activated charcoal (14 g) was added to the resulting organic fraction, and the mixture was stirred at ambient temperature overnight. The mixture was filtered on Celite, using methyl t-butyl ether for rinsing. The filtrate was dried over MgSO₄, filtered, and concentrated to a volume of 90 mL using a rotary evaporator. This solution of crude acetic acid 3{(1S,2S)-2-acetoxy-1-benzyl-3-[(2S)-2-ethylcarbamoyl-4,4-difluoro-3,3-dimethyl-pyrrolidin-1-yl]-3-oxo-propylcarbamoyl}2,5-dimethyl-phenyl ester was carried directly to the next step. Analytical data was obtained by concentrating a sample of this solution: m.p.=88-93° C.; ¹H NMR (300 MHz, DMSO-d₆) displayed a 10:1 mixture of rotamers. Major rotamer resonances: δ 8.58 (d, J=8.2 Hz, 1H), 8.02 (t, J=7.5 Hz, 1H), 7.18-7.42 (m, 5H), 6.92 (s, 1H), 6.84 (s, 1H), 5.34 (d, J=3.2 Hz, 1H), 4.41-4.66 (m, 2H), 4.19-4.32 (m, 2H), 3.03-3.26 (m, 2H), 2.95 (app dd, J=2.4, 13.8 Hz, 1H), 2.78 (app dd, J=11.7, 13.8 Hz, 1H), 2.27 (s, 3H), 2.25 (s, 3H), 1.73 (s, 3H), 1.22 (br s, 3H), 1.07 (br s, 3H), 1.04 (t, J=7.2 Hz, 3H) [characteristic minor rotamer resonances: δ 7.76-7.87 (m), 6.72 (s), 5.46 (d, J=3.7 Hz), 2.07 (s), 1.79 (s)]; ¹³C NMR (75 MHz, DMSO-d₆) displayed a ˜10:1 mixture of rotamers. Major rotamer resonances: δ 170.5, 169.2, 169.0, 166.8, 166.7, 149.4, 139.1, 138.8, 136.1, 129.7, 128.3, 127.8 (dd, J_{CF}=251.2, 254.9 Hz), 126.5, 125.7, 124.7, 123.9, 73.3, 68.2, 51.4, 43.9 (t, J_{CF}=20.5 Hz), 33.8, 33.4, 22.0 (d, J_{CF}=6.0 Hz), 20.8, 20.5, 17.6 (d, J_{CF}=7.0 Hz),15.0, 12.2 [characteristic minor rotamer resonances: δ 169.5, 168.9, 167.0, 149.5, 138.7, 129.3, 128.5, 125.4, 124.8, 124.2, 34.1, 21.2, 14.7]; MS (CI) m/z 616.2859 (616.2834 calcd for C₃₂H₄₀N₃O₇F₂, M+H⁺); elemental analysis calcd for C₃₂H₃₉F₂N₃O₇: C, 62.43; H, 6.38; N, 6.83; F, 6.17; found: C, 62.08; H, 6.68; N, 6.53; F, 5.85.

### Example 14

### Preparation of (2S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic Acid Ethylamide

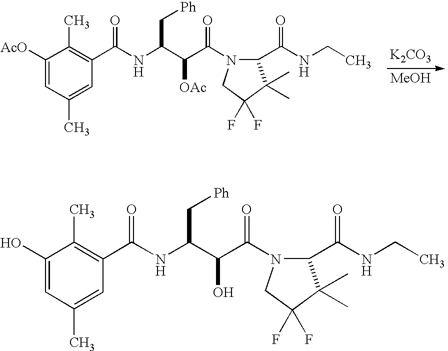

Methanol (30.0 mL) and K₂CO₃ (7.16 g, 51.7 mmol) were added to the methyl t-butyl ether solution of acetic acid 3{(1S,2S)-2-acetoxy-1-benzyl-3-[(2S)-2-ethylcarbamoyl-4,4-difluoro-3,3-dimethyl-pyrrolidin-1-yl]-3-oxo-propylcarbamoyl}-2,5-dimethyl-phenyl ester (from above) at ambient temperature. After stirring for 2 h, the resulting yellow solution was diluted with ethyl acetate (140 mL), 1 N HCl (50 mL), and 0.5 N HCl (140 mL), and the layers were then separated. The resulting organic fraction was sequentially washed with saturated aqueous NaHCO₃ (90 mL), 0.5 N HCl (70 mL), H₂O (140 mL), and saturated aqueous NaCl (70 mL). The organic fraction was then concentrated to a volume of −100 mL by distillation at one atmosphere, and the resulting solution was then cooled to ambient temperature. Diisopropyl ether (190 mL) was slowly added, and the resulting crystalline suspension was stirred overnight at ambient temperature. The suspension was filtered, using diisopropyl ether (50 mL) for rinsing. After drying under vacuum, 9.88 g (79.1%) of (2S)-4,4-difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide was obtained as a white solid: m.p.=208-214° C.; ¹H NMR (300 MHz, DMSO-d₆) displayed a ˜9:1 mixture of rotamers. Major rotamer resonances: δ 9.21 (s, 1H), 8.07 (d, J=8.2 Hz, 1H), 7.90 (t, J=5.5 Hz, 1H), 7.15-7.39 (m, 5H), 6.62 (s, 1H), 6.40 (s, 1H), 5.45 (d, J=6.3 Hz, 1H), 3.954.50 (m, 5H), 3.02-3.22 (m, 2H), 2.89 (app dd, J=2.0, 13.5 Hz, 1H), 2.72 (app dd, J=10.4, 13.4 Hz, 1H), 2.17 (s, 3H), 1.78 (s, 3H), 1.22 (s, 3H), 1.05 (s, 3H), 1.03 (t, J=7.2 Hz, 3H) [characteristic minor rotamer resonances: δ 6.15 (d, J=8.7 Hz), 7.85 (t, J=5.7 Hz), 6.34 (s), 5.31 (d, J=7.6 Hz), 4.73 (s), 1.81 (s); ¹³C NMR (75 MHz, DMSO-d₆) displayed a ˜9:1 mixture of rotamers. Major rotamer resonances: δ 171.0, 169.6, 167.2, 155.5, 139.7, 139.1, 135.1, 129.8, 128.2, 128.1 (dd, J_{CF}=251.4, 254.0 Hz), 126.2, 118.7, 118.6, 116.2, 72.8, 68.5, 53.1, 51.5 (t, J_{CF}=32.0 Hz), 43.7 (t, J_{CF}=20.5 Hz), 34.2, 33.8, 22.5 (d, J_{CF}=4.7 Hz), 20.9, 17.4 (d, J_{CF}=7.3 Hz), 15.1, 12.2 [characteristic minor rotamer resonances: δ 171.8, 169.7, 168.0, 138.8, 129.5, 23.1, 14.9; MS (CI) m/z 532.2614 (532.2623 calcd for C₂₈H₃₆N₃O₅F₂, M+H⁺); elemental analysis calc for C₂₈H₃₅F₂N₃O₅: C, 63.26; H, 6.64; N, 7.90; F, 7.15; found: C, 63.20; H, 6.67; N, 7.87; F, 7.07.

### Example 15

### Preparation of Crystalline (2S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic Acid Ethylamide

Amorphous (2S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide was allowed to stir with water (30 mg of compound per mL of water), in the form of a slurry, at a temperature of from about 50° C. to about 75° C., for about 6 hours to about 48 hours. The slurry was then cooled to room temperature and filtered. The remaining solid was dried in a vacuum oven between about 30° C. to about 60° C. for about 2 hours to about 24 hours under an atmospheric pressure of about 30 psi.

### Example 16

### X-ray Diffraction Pattern for Crystalline (2S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic Acid Ethylamide

Powder X-ray diffraction patterns for (2S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide were collected using a Bruker AXS D8 Advance diffractometer X-ray equipped with a Cu X-ray source operated at 40 kV and 50 mA. During analysis the samples were rotated at 60 rpm and analyzed from angles of 4°-40° (θ-2θ).

Samples (approximately 100 mg) were packed in Lucite sample cups fitted with Si (511) plates as the bottom of the cup to give no background signal. Samples were spun in the (p plane at a rate of 30 rpm to minimize crystal orientation effects. The x-ray source (KCu_{α}, λ=1.54 Å) was operated at a voltage of 45 kV and a current of 40 mA. Data for each sample were collected over a period of about 1 to 2 minutes in continuous detector scan mode at a scan speed of 1.8 seconds/step and a step size of 0.04°/step. Diffractograms were collected over the 2θ range of 4° to 40°. The results are summarized in table 2.

TABLE 2

| | Intensity |
|---|---|
| Angle 2-Theta | (% of highest)* |
| 6.6 | 2.16 |
| 7.4 | 2.45 |
| 8.2 | 23.59 |
| 8.6 | 100.00 |
| 10.4 | 2.95 |
| 10.5 | 2.96 |
| 11.1 | 17.12 |
| 12.0 | 12.93 |
| 13.2 | 4.75 |
| 13.8 | 6.70 |
| 14.7 | 26.58 |
| 15.5 | 16.36 |
| 16.4 | 17.29 |
| 17.0 | 18.85 |
| 17.8 | 16.20 |
| 18.4 | 20.43 |
| 19.0 | 11.56 |
| 19.8 | 13.94 |
| 20.7 | 16.74 |
| 21.5 | 8.42 |
| 22.2 | 9.79 |
| 23.5 | 7.35 |
| 24.1 | 9.76 |
| 24.4 | 6.75 |
| 25.2 | 7.94 |
| 26.1 | 5.89 |
| 26.5 | 5.56 |
| 26.9 | 4.91 |
| 27.2 | 8.76 |
| 27.8 | 5.01 |
| 28.0 | 5.32 |
| 32.0 | 4.99 |

*The peak intensity may change depending on the crystalline size and habit

### Example 17

### Raman Scattering Spectra of Crystalline (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic Acid (2,2,2-trifluoroethyl)-amide

Raman scattering spectra of crystalline (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoro-ethyl)-amide were collected using Depressive Raman Spectrum from Kaiser Optical Instruments, (Raman RXN1) equipped with a base unit contains the laser (NIR laser Diode operated at wavelength of 758 nm external-cavity-stabilized diode laser, the spectrograph, 2-D array detector Charge-Coupled Device (CCD). During the analysis, the light from the laser was coupled into a multi-mode optical fiber, which carries the laser excitation at 785 nm to a fiber optic probe. Emission fiber optic cable was filtered out at the probe head, and the laser light was focused onto the sample. The backscattered from the sample was filtered to remove the light at the laser wavelength and was sent to the spectrograph. The spectrograph removed any residual laser light and dispersed the Raman light into charge-coupled device (CCD) detector. During the analysis the sample was analyzed from 0-3450 cm⁻¹. Samples (approximately 2-10 mg) were placed on a glass plate. Data was collected over a period of about 15 to 120 seconds. The resolution was 4 cm⁻¹. Diffractograms were collected and the results are summarized below.

| Raman Shift (cm⁻¹) | % Intensity |
|---|---|
| 518 | 26 |
| 540 | 29 |
| 599 | 44 |
| 760 | 39 |
| 838 | 41 |
| 1004 | 100 |
| 1079 | 48 |
| 1475 | 26 |
| 1715 | 19 |

### Example 18

### Raman Scattering Spectra of Crystalline (2S)-4,4-Difluoro-1-[(2S,3S)-2-hydroxy-3-(3-hydroxy-2,5-dimethyl-benzoylamino)-4-phenyl-butyryl-3,3-dimethyl-pyrrolidine-2-carboxylic Acid Ethylamide

Raman scattering spectra of crystalline (2S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide were collected using Depressive Raman Spectrum from Kaiser Optical Instruments, (Raman RXN1) equipped with a base unit contains the laser (NIR laser Diode operated at wavelength of 758 nm external-cavity-stabilized diode laser, the spectrograph, 2-D array detector Charge-Coupled Device (CCD). During the analysis the light from the laser was coupled into a multi-mode optical fiber, which carried the laser excitation at 785 nm to a fiber optic probe. Emission fiber optic cable was filtered out at the probe head, and the laser light was focused onto the sample. The backscattered from the sample was filtered to remove the light at the laser wavelength and was sent to the spectrograph. The spectrograph removed any residual laser light and dispersed the Raman light into charge-coupled device (CCD) detector. During the analysis the samples were analyzed from 0-3450 cm⁻¹. Samples (approximately 2-10 mg) were placed on a glass plate Data for each sample was collected over a period of about 15 to 120 seconds. The resolution was 4 cm⁻¹. Diffractograms were collected and the results are summarized below.

| Raman Shift (cm⁻¹) | % Intensity |
|---|---|
| 463 | 64 |
| 555 | 32 |
| 622 | 35 |
| 655 | 27 |
| 753 | 32 |
| 781 | 31 |
| 899 | 31 |
| 976 | 24 |
| 1002 | 100 |
| 1032 | 33 |
| 1320 | 46 |

While the invention has been illustrated by reference to specific and preferred embodiments, those skilled in the art will recognize that variations and modifications may be made through routine experimentation and practice of the invention. Thus, the invention is intended not to be limited by the foregoing description, but to be defined by the appended claims and their equivalents.

1. A method of preparing compounds of formula (I): 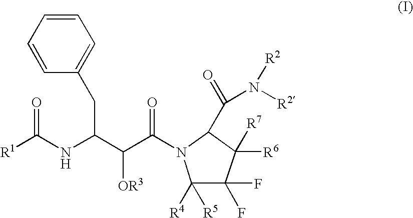
wherein:
R¹ is phenyl optionally substituted by at least one substituent independently chosen from C₁₋₆ alkyl, hydroxyl, C₁₋₆ alkylcarbonyloxy, C₆₋₁₀ arylcarbonyloxy, and heteroarylcarbonyloxy;
R² is C₂₋₆ alkenyl or C₁₋₆ alkyl optionally substituted with at least one halogen;
R^{2′} is H or C₁-C₄ alkyl;
R³ is a hydroxyl protecting group; and
R⁴, R⁵, R⁶ and R⁷ are independently chosen from H and C₁-C₆ alkyl;
comprising:
reacting a compound of formula (II), wherein Y¹ is hydroxyl or a leaving group, with a compound of formula (III), or a salt or solvate thereof,

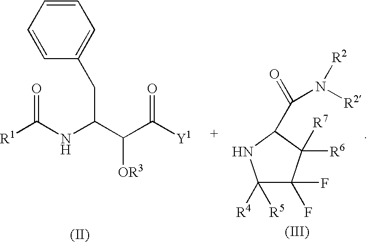

2. The method of claim 1, wherein in the compound of formula (I):
R³ is C₁₋₆ alkylcarbonyl, C₆₋₁₀ arylcarbonyl, or heteroarylcarbonyl;
R⁴ and R⁵ are each hydrogen; and
R⁶ and R⁷ are independently chosen from hydrogen and methyl.

3. The method of claim 2, wherein in the compound of formula (I) R^{2′} is H.

4. The method of claim 3, wherein in the compound of formula (I):
R¹ is phenyl substituted with at least one substituent independently chosen from methyl, hydroxyl, C₁₋₄ alkylcarbonyloxy, C₆₋₁₀ arylcarbonyloxy, and heteroarylcarbonyloxy; and
R⁶ and R⁷ are methyl.

5. The method of claim 4, wherein in the compound of formula (I):
R² is C₂₋₆ alkenyl or C₁₋₄ alkyl optionally substituted with at least one fluorine; and
R³ is C₁₋₆ alkylcarbonyl.

6. The method of claim 5, wherein in the compound of formula (I) R² is C₁₋₆ alkyl optionally substituted with at least one fluorine.

7. The method of claim 6, wherein in the compound of formula (I) R¹ is phenyl substituted with at least one substituent independently chosen from methyl, hydroxyl, and methylcarbonyloxy.

8. The method of claim 6, wherein in the compound of formula (I):
R² is —CH₂CF₃; and
R³ is methylcarbonyl.

9. The method of claim 4, wherein in the compound of formula (I) R¹ is phenyl substituted with at least one substituent independently chosen from methyl and methylcarbonyloxy.

10. The method of claim 9, wherein the compound of formula (I) is:

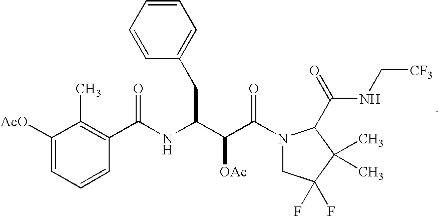

11. Crystalline (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide, or a pharmaceutically acceptable salt or solvate thereof.

12. A crystalline form of (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide according to claim 11, exhibiting a characteristic peak in the powder x-ray diffraction pattern, expressed in degrees two-theta, at about 8.7.

13. A crystalline form of (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide according to claim 11, exhibiting a melting temperature of between about 191° C. and about 200° C.

14. A crystalline form of (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide according to claim 11, that exhibits a peak in the Raman scattering spectrum, expressed in Raman shift, at about 1004 cm⁻¹.

15. A method of preparing a crystalline form of (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide, comprising:
a) deprotecting the compound of formula (I-C), 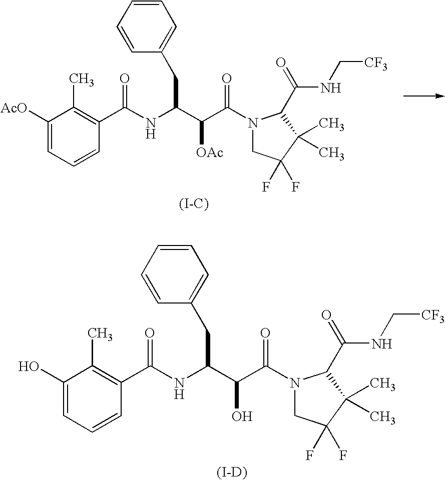
to afford amorphous (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide (1-D); and
b) slurrying amorphous (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2trifluoroethyl)-amide in water to afford a crystalline form of (2S)-4,4-difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2,2-trifluoroethyl)-amide.
